SEQUENCE LISTING (29 sequences)

SEQ ID NO: 1 - DNA, 516 bp, synthetic construct
  source 1..516: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcgagacggtcgggtccagatattcgtatctgtcgagtagagtgtgggctcgtggccgcggtcggcgtggactgtagaacactgccaatgccggtcccaagcccggataaaagtggagggtacagtccacgctctagagcggacttcggtccgctttt

SEQ ID NO: 2 - DNA, 577 bp, synthetic construct
  source 1..577: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactagtcgacgggccgcactcgccggtcccaagcccggataaaatgggagggggcgggaaaccgcctaaccatgccgagtgcggccgcttgccatgtgtatcggtccggagacggtcgggtccagatattcgtatctgtcgagtagagtgtgggctccggtccgatactctgatgatgggtcccatcattcatggcaagtggccgcggtcggcgtggactgtagaacactgccaatgccggtcccaagcccggataaaagtggagggtacagtccacgctctagagcgcacttcggtgcgctttt

SEQ ID NO: 3 - RNA, 42 nt, synthetic construct
  source 1..42: mol_type = other RNA
aatttctactcttgtagatctaggaatattgaagggggcagg

SEQ ID NO: 4 - RNA, 48 nt, synthetic construct
  source 1..48: mol_type = other RNA
asaststtctactcttgtagatctaggaatattgaagggggcsasgsg

SEQ ID NO: 5 - RNA, 42 nt, synthetic construct; identical to SEQ ID NO: 3
  source 1..42: mol_type = other RNA

SEQ ID NO: 6 - RNA, 84 nt, synthetic construct
  source 1..84: mol_type = other RNA
aatttctactcttgtagatctaggaatattgaagggggcaggaatttctactcttgtagatctaggaatattgaagggggcagg

SEQ ID NO: 7 - RNA, 52 nt, synthetic construct
  source 1..52: mol_type = other RNA
aactttaaataatttctactcttgtagatctaggaatattgaagggggcagg

SEQ ID NO: 8 - RNA, 62 nt, synthetic construct
  source 1..62: mol_type = other RNA
aactttaaataactttaaataatttctactcttgtagatctaggaatattgaagggggcagg

SEQ ID NO: 9 - RNA, 72 nt, synthetic construct
  source 1..72: mol_type = other RNA
aactttaaataactttaaataactttaaataatttctactcttgtagatctaggaatattgaagggggcagg

SEQ ID NO: 10 - RNA, 61 nt, synthetic construct
  source 1..61: mol_type = other RNA
aatttctactcttgtagataatttctactcttgtagatctaggaatattgaagggggcagg

SEQ ID NO: 11 - RNA, 95 nt, synthetic construct
  source 1..95: mol_type = other RNA
tcaaggcactcttgcccacgccaccagctccaactaccacaagtttatatgttgtggaaggtccagttttgggggctattacaacattmcmgmat

SEQ ID NO: 12 - RNA, 92 nt, synthetic construct
  source 1..92: mol_type = other RNA
tcaaggcactcttgcccacgccaccagctccaactaccacaagtttatatgttgtggaaggtccagttttgggggctattacaacattcgat

SEQ ID NO: 13 - RNA, 110 nt, synthetic construct
  source 1..110: mol_type = other RNA
tcaaggcactcttgcccacgccaccagctccaactaccacaagtttatatgttgtggaaggtccagttttgggggctattacaacattmcmgmattcaagacgatcgcat

SEQ ID NO: 14 - RNA, 107 nt, synthetic construct
  source 1..107: mol_type = other RNA
tcaaggcactcttgcccacgccaccagctccaactaccacaagtttatatgttgtggaaggtccagttttgggggctattacaacattcgattcaagacgatcgcat

SEQ ID NO: 15 - RNA, 140 nt, synthetic construct
  source 1..140: mol_type = other RNA
tcaaggcactcttgcccacgccaccagctccaactaccacaagtttatatgttgtggaaggtccagttttgggggctattacaacattmcmgmattcaagacgatcgcatttgcgccatcttatccaagaagtactgtat

SEQ ID NO: 16 - RNA, 137 nt, synthetic construct
  source 1..137: mol_type = other RNA
tcaaggcactcttgcccacgccaccagctccaactaccacaagtttatatgttgtggaaggtccagttttgggggctattacaacattcgattcaagacgatcgcatttgcgccatcttatccaagaagtactgtat

SEQ ID NO: 17 - RNA, 110 nt, synthetic construct
  source 1..110: mol_type = other RNA
atcgacaagaaagggactgagttttagagctagaaatagcaagttaaaataaggctagtccgttatcaacttgaaaaagtggcaccgagtcggtgctttttttttttttt

SEQ ID NO: 18 - RNA, 115 nt, synthetic construct
  source 1..115: mol_type = other RNA
atcgacaagaaagggactgagttttagagctagaaatagcaagttaaaataaggctagtccgttatcaacttgaaaaagtggcaccgagtcggtgcttttttttttttttttttt

SEQ ID NO: 19 - RNA, 120 nt, synthetic construct
  source 1..120: mol_type = other RNA
atcgacaagaaagggactgagttttagagctagaaatagcaagttaaaataaggctagtccgttatcaacttgaaaaagtggcaccgagtcggtgctttttttttttttttttttttttt

SEQ ID NO: 20 - RNA, 118 nt, synthetic construct
  source 1..118: mol_type = other RNA
accatcgacaagaaagggactgagttttagagctagaaatagcaagttaaaataaggctagtccgttatcaacttgaaaaagtggcaccgagtcggtgcttttttttttttttttttt

SEQ ID NO: 21 - RNA, 115 nt, synthetic construct
  source 1..115: mol_type = other RNA
atcgacaagaaagggactgagttttagagctagaaatagcaagttaaaataaggctagtccgttatcaacttgaaaaagtggcaccgagtcggtgcttttatcgcgtgctctcgt

SEQ ID NO: 22 - RNA, 118 nt, synthetic construct
  misc_feature 1: note = other RNA
  source 1..118: mol_type = other RNA
accatcgacaagaaagggactgagttttagagctagaaatagcaagttaaaataaggctagtccgttatcaacttgaaaaagtggcaccgagtcggtgcttttatcgcgtgctctcgt

SEQ ID NO: 23 - DNA, 322 bp, synthetic construct
  source 1..322: mol_type = other DNA
ggccgcactcgccggtcccaagcccggataaaatgggagggggcgggaaaccgcctaaccatgccgagtatgcatggaacacaaagcatagactgcgttttagagctagaaatagcaagttaaaataaggctagtccgttatcaacttgaaaaagtggcaccgagtcggtgctttaaaacaacaacccaaccaacaaaccaacaccaaacaacaaaccaaaccccaacaaacaaccaccaacccagaattctcggcgtggactgtagaacactgccaatgccggtcccaagcccggataaaagtggagggtacagtccacgc

SEQ ID NO: 24 - DNA, 244 bp, synthetic construct
  source 1..244: mol_type = other DNA
ccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaacc

SEQ ID NO: 25 - DNA, 190 bp, synthetic construct
  source 1..190: mol_type = other DNA
aaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacgg

SEQ ID NO: 26 - DNA, 160 bp, synthetic construct
  source 1..160: mol_type = other DNA
cctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattgcgtca

SEQ ID NO: 27 - DNA, 113 bp, synthetic construct
  source 1..113: mol_type = other DNA
cagagatgttttcttgggttaattgaggcctgagtataaggtgacttatacttgtaatctatctaaacggggaacctctctagtagacaatcccgtgctaaattgtaggacta

SEQ ID NO: 28 - DNA, 190 bp, synthetic construct
  source 1..190: mol_type = other DNA
gggagaccctcgaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgct

SEQ ID NO: 29 - DNA, 123 bp, synthetic construct
  source 1..123: mol_type = other DNA
ttgggttaattgaggcctgagtataaggtgacttatacttgtaatctatctaaacggggaacctctctagtagacaatcccgtgctaaattgtaggactaattccatttatcagatttctaga

(19) United States
(12) Patent Application Publication
(10) Pub. No.: US 2024/0376468 A1
(43) Pub. Date: Nov. 14, 2024

(54) CIRCULAR GUIDE RNAs FOR CRISPR/CAS EDITING SYSTEMS

(71) Applicant: Beam Therapeutics Inc., Cambridge, MA (US)

(72) Inventors: Brian CAFFERTY, Cambridge, MA (US); Fei RAN, Cambridge, MA (US); Bernd ZETSCHE, Cambridge, MA (US); Justin THOMAS, Cambridge, MA (US); Maria MONTIEL, Cambridge, MA (US); Krishna SAPKOTA, Cambridge, MA (US)

(21) Appl. No.: 18/526,492

(22) Filed: Dec. 1, 2023

Related U.S. Application Data
(63) Continuation of application No. PCT/US2022/032028, filed on Jun. 2, 2022.
(60) Provisional application No. 63/196,039, filed on Jun. 2, 2021.
(60) Provisional application No. 63/305,128, filed on Jan. 31, 2022.

(51) Int. Cl. C12N 15/11; C12N 9/00; C12N 9/22; C12P 19/34

(52) U.S. Cl. CPC C12N 15/111; C12N 9/22; C12N 9/93; C12P 19/34; C12N 2310/20; C12Y 605/01003

(57) ABSTRACT

The present invention provides, among other things, circular guide RNA (cgRNA) compositions and methods for making same. For example, in some aspects, a method of making egRN A is provided comprising enzymatically ligating two ends of a linear guide RNA, creating a cgRNA. In some aspects, a. method is provided for making cgRNA comprising contacting a linear guide RNA with a ligating enzyme, wherein the contacting brings together a first end and a second end of the guide RN A. and wherein the ligating enzyme ligates the first and the second end of the guide RNA thus creating a cgRNA. In some aspects, circularization is carried out by self-splicing introns. In some aspects, provided are circular RNA compositions, for example, circular messenger RNA, and methods for making same.

### CROSS-REFERENCE TO RELATED APPLICATIONS

This application claims priority to U.S. Provisional Patent Applications Ser. No. 63/196,039 filed Jun. 2, 2021 and 63/305,128 filed Jan. 31, 2022, the contents of which are incorporated by reference herein in entirety for all purposes.

### INCORPORATION-BY-REFERENCE OF SEQUENCE LISTING

The contents of the file named “BEM-010US1_SL.xml”, which was created on Aug. 5, 2024, and is 30,639 bytes in size, are hereby incorporated by reference in their entirety.

### BACKGROUND

CRISPR/Cas editing systems include the use of guide RNA molecules (gRNA) in association with Cas endonucleases, and related enzymes, for applications in gene editing as well as related systems, including base editing, and other editing applications. Briefly, one or more gRNA molecules assembles with a Cas protein in a complex and guides the ribonucleic acid complex (RNP) to specific DNA (for example, in Cas9 and Cas12 systems) and/or RNA (for example, in Cas13 systems) sequences.

A common form of gRNA used for therapeutic applications are single, non-natural RNAs of approximately 100 nucleotides that form ribonucleoproteins with Cas proteins such as Cas9. Some of the problems facing gRNA manufacturing include poor purity and yield, for example, chemical synthesis and purification typically results in only about 70% purity with only about 3% yield. The ability to adapt CRISPR/Cas editing systems to new technologies (e.g., gene editing) requires that guide RNAs (gRNAs) persist long enough within target cells to enable desired editing. Degradation of gRNA by exonuclease is a significant challenge to achieving desired editing. For this reason, chemical modifications are typically installed at the 5′ and 3′ ends of the gRNA which has been shown to greatly increase editing, particularly when the CRISPR/Cas-based editing system is delivered via mRNA and gRNA. Typically, plasmid DNA and solid-phase synthesis using phosphoramidite chemistry are typical approaches to obtain therapeutic sgRNAs. While incorporation of modifications can both increase the chemical stability of sgRNA and reduce editing of genomic DNA at undesired locations (off-targets), some chemical modifications can, however, be cytotoxic. The need for end modifications also precludes total enzymatic synthesis of gRNA.

### SUMMARY OF THE INVENTION

Provided herein are methods, compositions and kits to enhance the stability of gRNA for use in CRISPR-Cas systems. The invention provides, in some aspects, methods to produce circular gRNA (cgRNA) that has increased stability against ubiquitous cellular exonucleases increasing frequency of successful editing events. In other aspects, also provided herein are methods to produce other circular RNA, e.g. circular messenger RNA, circular long non-coding RNA, etc. Methods of producing circular RNA comprise use of enzymatic or chemical ligation, and/or the use of ribozymes for circularization. The resultant purity, yield and integrity of the cgRNA allows for increased editing efficiencies and a reduction of off-target editing. The invention provides, in some aspects, compositions and kits comprising cgRNA with improved stability and increased editing efficiencies with reduced off-target editing. Circular guide RNA (cgRNA) is at least as stable as end modified gRNA without requiring non-natural modifications. Further, since cgRNA made synthetically will only cyclize full-length product, it is easily isolated and results in high yields of pure product. By use of enzymatic methods, purity is increased by minimizing residual chemical carryover, and reducing manufacturing costs.

In one aspect, a method is provided that uses RNA ligation to circularize synthetic RNA or transcribed RNA. In some aspects, the method comprises cyclizing a linear RNA with a ligating enzyme, wherein the ligating enzyme ligates the first and the second end of the guide RNA thus creating a circular RNA. In some aspects, the method comprises cyclizing a linear guide RNA with a ligating enzyme, wherein the ligating enzyme ligates the first and the second end of the guide RNA thus creating a circular guide RNA. In some aspects, the method comprises cyclizing a linear messenger RNA with a ligating enzyme, wherein the ligating enzyme ligates the first and the second end of the messenger RNA thus creating a circular messenger RNA. In some aspects, the method comprises cyclizing a linear long non-coding RNA with a ligating enzyme, wherein the ligating enzyme ligates the first and the second end of the long non-coding RNA thus creating a circular long non-coding RNA.

In another aspect, a method is provided that uses a circularization technique that employs ribozymes and ligases.

In another aspect, cgRNA is synthesized using chemical ligation, including, for example, click chemistry, copper azide cycloaddition chemistry, thiol-ene, native chemical ligation and others. Accordingly, in some embodiments, the cgRNA is synthesized using click chemistry. In some embodiments, the cgRNA is synthesized using copper azide cycloaddition chemistry. In some embodiments, the cgRNA is synthesized using thiol-ene. In some embodiments, the cgRNA is synthesized using native chemical ligation.

In some embodiments, the method comprises a ligating enzyme selected from the group consisting of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, Splint® Ligase E. coli DNA Ligase, 9°N DNA Ligase, CircLigase, CircLigase II, DNA Ligase I, DNA Ligase III, and DNA Ligase IV. Accordingly, in some embodiments, the ligating enzyme is T4 RNA ligase 1. In some embodiments, the ligating enzyme is T4 RNA ligase 2. In some embodiments, the ligating enzyme is RtcB Ligase. RtcB is a family of RtcB ligases, including for example, E. coli RtcB, Human RtcB among others. In some embodiments, the ligating enzyme is Thermo-stable 5′ App DNA/RNA Ligase. In some embodiments, the ligating enzyme is ElectroLigase. In some embodiments, the ligating enzyme is T4 DNA Ligase. In some embodiments, the ligating enzyme is T3 DNA Ligase. In some embodiments, the ligating enzyme is T7 DNA Ligase. In some embodiments, the ligating enzyme is Taq DNA Ligase. In some embodiments, the ligating enzyme is Splint® Ligase E. coli DNA Ligase. In some embodiments, the ligating enzyme is 9°N DNA Ligase. In some embodiments, the ligating enzyme is CircLigase. In some embodiments, the ligating enzyme is CircLigase II. In some embodiments, the ligating enzyme is DNA Ligase I. In some embodiments, the ligating enzyme is DNA Ligase III. In some embodiments, the ligating enzyme is DNA Ligase IV.

In some embodiments, the ligation-based method comprises using two or more partially complementary synthetic RNAs that are subsequently ligated (“template approach”), and methods that do not require complementarity between two or more synthetic RNAs (“non-templated approach”).

In some embodiments, the ligating occurs in the absence of a template between the first end and the second end of the RNA. In some embodiments, the ligating occurs in the absence of a template between the first end and the second end of the guide RNA. In some embodiments, the ligating occurs in the absence of a template between the first end and the second end of the messenger RNA.

In some embodiments, the first end and the second end of the RNA has partial complementarity. In some embodiments, the first end and the second end of the guide RNA has partial complementarity. In some embodiments, the first end and the second end of the messenger RNA has partial complementarity.

In some embodiments, the partial complementarity between the first end and the second end comprises complementarity of about 5-30 nucleotides, about 5-25 nucleotides, about 5-20 nucleotides, about 5-15 nucleotides, about 5-10 nucleotides, about 10-25 nucleotides, about 10-20 nucleotides, about 10-15 nucleotides, about 15-25 nucleotides, about 15-20 nucleotides, about 20-25 nucleotides, about 25-30 nucleotides.

In some embodiments, the partial complementarity between the first end and the second end comprises complementarity of about 5 nucleotides, about 10 nucleotides, about 12 nucleotides, about 14 nucleotides, about 16 nucleotides, about 18 nucleotides, about 20 nucleotides, about 22 nucleotides, about 24 nucleotides, about 26 nucleotides, about 28 nucleotides, about 30 nucleotides.

In some embodiments, the partial complementarity between the first end and the second end comprises complementarity of about 30-100 nucleotides, about 30-90 nucleotides, about 30-80 nucleotides, about 30-75 nucleotides, about 30-70 nucleotides, about 50-100 nucleotides, about 50-90 nucleotides, about 50-80 nucleotides, about 50-75 nucleotides, about 50-70 nucleotides or about 50-60 nucleotides.

In some embodiments, the partial complementarity between the first end and the second end comprises complementarity of about 30 nucleotides, about 40 nucleotides, about 50 nucleotides, about 60 nucleotides, about 70 nucleotides, about 80 nucleotides, about 90 nucleotides or about 100 nucleotides.

In some embodiments, the method comprises a ligating enzyme wherein the ligating occurs in the absence of an oligonucleotide splint. In some embodiments, the ligating occurs in the absence of a template. In some embodiments, the ligating enzyme is a single-stranded ligase. In some embodiments, the single-stranded ligase is a T4 RNA ligase 1.

In some embodiments, the ligating occurs in the presence of a template. In some embodiments, the template is an oligonucleotide splint.

In some embodiments, one or more oligonucleotide splints anneal to the first end and to the second end of the guide RNA thereby facilitating the formation of a loop structure between the first end and the second end of the guide RNA.

In some embodiments, the oligonucleotide splint is a DNA splint. In some embodiments, wherein the oligonucleotide splint is an RNA splint.

In some embodiments, the oligonucleotide splint has about 10-50 nucleotides, about 10-45 nucleotides, about 10-40 nucleotides, about 10-35 nucleotides, about 10-30 nucleotides, about 10-25 nucleotides, about 10-20 nucleotides, about 10-15 nucleotides, about 15-50 nucleotides, about 15-45 nucleotides, about 15-40 nucleotides, about 15-35 nucleotides, about 15-30 nucleotides, about 15-25 nucleotides, about 15-20 nucleotides, about 20-50 nucleotides, about 20-45 nucleotides, about 20-40 nucleotides, about 20-35 nucleotides, about 20-30 nucleotides, about 20-25 nucleotides, about 25-50 nucleotides, about 25-45 nucleotides, about 25-40 nucleotides, about 25-35 nucleotides, about 25-30 nucleotides, about 30-50 nucleotides, about 30-45 nucleotides, about 30-40 nucleotides, about 30-35 nucleotides, about 35-50 nucleotides, about 35-45 nucleotides, about 35-40 nucleotides, about 40-50 nucleotides, or about 45-50 nucleotides. In some embodiments, the oligonucleotide splint has between about 15-40 nucleotides.

In some embodiments, the oligonucleotide splint is about 90% of the gRNA. In some embodiments, the oligonucleotide splint is about 85% of the gRNA. In some embodiments, the oligonucleotide splint is about 80% of the gRNA. In some embodiments, the oligonucleotide splint is about 75% of the gRNA. In some embodiments, the oligonucleotide splint is about 70% of the gRNA. In some embodiments, the oligonucleotide splint is about 65% of the gRNA. In some embodiments, the oligonucleotide splint is about 60% of the gRNA. In some embodiments, the oligonucleotide splint is about 55% of the gRNA. In some embodiments, the oligonucleotide splint is about 50% of the gRNA. In some embodiments, the oligonucleotide splint is about 45% of the gRNA. In some embodiments, the oligonucleotide splint is about 40% of the gRNA. In some embodiments, the oligonucleotide splint is about 35% of the gRNA. In some embodiments, the oligonucleotide splint is about 30% of the gRNA. In some embodiments, the oligonucleotide splint is about 25% of the gRNA. In some embodiments, the oligonucleotide splint is about 20% of the gRNA. In some embodiments, the oligonucleotide splint is about 15% of the gRNA. In some embodiments, the oligonucleotide splint is about 10% of the gRNA. In some embodiments, the oligonucleotide splint is about 5% of the gRNA. In some embodiments, the oligonucleotide splint is about 2% of the gRNA. In some embodiments, the oligonucleotide splint is about 1% of the gRNA.

In some embodiments, the oligonucleotide splint is about 90% of the mRNA. In some embodiments, the oligonucleotide splint is about 85% of the mRNA. In some embodiments, the oligonucleotide splint is about 80% of the mRNA. In some embodiments, the oligonucleotide splint is about 75% of the mRNA. In some embodiments, the oligonucleotide splint is about 70% of the mRNA. In some embodiments, the oligonucleotide splint is about 65% of the mRNA. In some embodiments, the oligonucleotide splint is about 60% of the mRNA. In some embodiments, the oligonucleotide splint is about 55% of the mRNA. In some embodiments, the oligonucleotide splint is about 50% of the mRNA. In some embodiments, the oligonucleotide splint is about 45% of the mRNA. In some embodiments, the oligonucleotide splint is about 40% of the mRNA. In some embodiments, the oligonucleotide splint is about 35% of the mRNA. In some embodiments, the oligonucleotide splint is about 30% of the mRNA. In some embodiments, the oligonucleotide splint is about 25% of the mRNA. In some embodiments, the oligonucleotide splint is about 20% of the mRNA. In some embodiments, the oligonucleotide splint is about 15% of the mRNA. In some embodiments, the oligonucleotide splint is about 10% of the mRNA. In some embodiments, the oligonucleotide splint is about 5% of the mRNA. In some embodiments, the oligonucleotide splint is about 2% of the mRNA. In some embodiments, the oligonucleotide splint is about 1% of the mRNA.

In some embodiments, the oligonucleotide splint is about 0.9% of the mRNA. In some embodiments, the oligonucleotide splint is about 0.8% of the mRNA. In some embodiments, the oligonucleotide splint is about 0.7% of the mRNA. In some embodiments, the oligonucleotide splint is about 0.6% of the mRNA. In some embodiments, the oligonucleotide splint is about 0.5% of the mRNA. In some embodiments, the oligonucleotide splint is about 0.4% of the mRNA. In some embodiments, the oligonucleotide splint is about 0.3% of the mRNA. In some embodiments, the oligonucleotide splint is about 0.2% of the mRNA. In some embodiments, the oligonucleotide splint is about 0.1% of the mRNA.

In some embodiments, the oligonucleotide splint hybridizes with about 5-50 nucleotides of the first end of the RNA. In some embodiments, the oligonucleotide splint hybridizes with about 5-45 nucleotides of the first end of the RNA. In some embodiments, the oligonucleotide splint hybridizes with about 5-40 nucleotides of the first end of RNA. In some embodiments, the oligonucleotide splint hybridizes with about 5-35 nucleotides of the first end of the RNA. In some embodiments, the oligonucleotide splint hybridizes with about 5-30 nucleotides of the first end of the RNA. In some embodiments, the oligonucleotide splint hybridizes with about 5-25 nucleotides of the first end of the RNA. In some embodiments, the oligonucleotide splint hybridizes with about 5-20 nucleotides of the first end of the RNA. In some embodiments, the oligonucleotide splint hybridizes with about 5-15 nucleotides of the first end of the RNA. In some embodiments, the oligonucleotide splint hybridizes with about 5-10 nucleotides of the first end of the RNA. In some embodiments, the oligonucleotide splint hybridizes with about 6-30 nucleotides of the first end of the RNA. In some embodiments, the RNA is messenger RNA. In some embodiments, the RNA is guide RNA.

In some embodiments, the oligonucleotide splint hybridizes with about 5-50 nucleotides of the first end of the guide RNA. In some embodiments, the oligonucleotide splint hybridizes with about 5-45 nucleotides of the first end of the guide RNA. In some embodiments, the oligonucleotide splint hybridizes with about 5-40 nucleotides of the first end of the guide RNA. In some embodiments, the oligonucleotide splint hybridizes with about 5-35 nucleotides of the first end of the guide RNA. In some embodiments, the oligonucleotide splint hybridizes with about 5-30 nucleotides of the first end of the guide RNA. In some embodiments, the oligonucleotide splint hybridizes with about 5-25 nucleotides of the first end of the guide RNA. In some embodiments, the oligonucleotide splint hybridizes with about 5-20 nucleotides of the first end of the guide RNA. In some embodiments, the oligonucleotide splint hybridizes with about 5-15 nucleotides of the first end of the guide RNA. In some embodiments, the oligonucleotide splint hybridizes with about 5-10 nucleotides of the first end of the guide RNA. In some embodiments, the oligonucleotide splint hybridizes with about 6-30 nucleotides of the first end of the guide RNA.

In some embodiments, 100% of the oligonucleotide splint hybridizes with the RNA. In some embodiments, 90% of the oligonucleotide splint hybridizes with the RNA. In some embodiments, 85% of the oligonucleotide splint hybridizes with the RNA. In some embodiments, 80% of the oligonucleotide splint hybridizes with the RNA. In some embodiments, 75% of the oligonucleotide splint hybridizes with the RNA. In some embodiments, 70% of the oligonucleotide splint hybridizes with the RNA. In some embodiments, 65% of the oligonucleotide splint hybridizes with the RNA. In some embodiments, 60% of the oligonucleotide splint hybridizes with the RNA. In some embodiments, 55% of the oligonucleotide splint hybridizes with the RNA. In some embodiments, 50% of the oligonucleotide splint hybridizes with the RNA. In some embodiments, 45% of the oligonucleotide splint hybridizes with the RNA. In some embodiments, 40% of the oligonucleotide splint hybridizes with the RNA. In some embodiments, 35% of the oligonucleotide splint hybridizes with the RNA. In some embodiments, 30% of the oligonucleotide splint hybridizes with the RNA. In some embodiments, 25% of the oligonucleotide splint hybridizes with the RNA. In some embodiments, 20% of the oligonucleotide splint hybridizes with the RNA. In some embodiments, 15% of the oligonucleotide splint hybridizes with the RNA. In some embodiments, 10% of the oligonucleotide splint hybridizes with the RNA. In some embodiments, 5% of the oligonucleotide splint hybridizes with the RNA. In some embodiments, 2% of the oligonucleotide splint hybridizes with the RNA. In some embodiments, 1% of the oligonucleotide splint hybridizes with the RNA. In some embodiments, the RNA is messenger RNA. In some embodiments, the RNA is guide RNA.

In some embodiments, 100% of the oligonucleotide splint hybridizes with the guide RNA. In some embodiments, 90% of the oligonucleotide splint hybridizes with the guide RNA. In some embodiments, 85% of the oligonucleotide splint hybridizes with the guide RNA. In some embodiments, 80% of the oligonucleotide splint hybridizes with the guide RNA. In some embodiments, 75% of the oligonucleotide splint hybridizes with the guide RNA. In some embodiments, 70% of the oligonucleotide splint hybridizes with the guide RNA. In some embodiments, 65% of the oligonucleotide splint hybridizes with the guide RNA. In some embodiments, 60% of the oligonucleotide splint hybridizes with the guide RNA. In some embodiments, 55% of the oligonucleotide splint hybridizes with the guide RNA. In some embodiments, 50% of the oligonucleotide splint hybridizes with the guide RNA. In some embodiments, 45% of the oligonucleotide splint hybridizes with the guide RNA. In some embodiments, 40% of the oligonucleotide splint hybridizes with the guide RNA. In some embodiments, 35% of the oligonucleotide splint hybridizes with the guide RNA. In some embodiments, 30% of the oligonucleotide splint hybridizes with the guide RNA. In some embodiments, 25% of the oligonucleotide splint hybridizes with the guide RNA. In some embodiments, 20% of the oligonucleotide splint hybridizes with the guide RNA. In some embodiments, 15% of the oligonucleotide splint hybridizes with the guide RNA. In some embodiments, 10% of the oligonucleotide splint hybridizes with the guide RNA. In some embodiments, 5% of the oligonucleotide splint hybridizes with the guide RNA. In some embodiments, 2% of the oligonucleotide splint hybridizes with the guide RNA. In some embodiments, 1% of the oligonucleotide splint hybridizes with the guide RNA.

In some embodiments, the oligonucleotide splint has perfect complementarity with 5-50 nucleotides of the first end of the RNA. In some embodiments, the oligonucleotide splint has perfect complementarity with 5-45 nucleotides of the first end of the RNA. In some embodiments, the oligonucleotide splint has perfect complementarity with 5-40 nucleotides of the first end of the RNA. In some embodiments, the oligonucleotide splint has perfect complementarity with 5-35 nucleotides of the first end of the RNA. In some embodiments, the oligonucleotide splint has perfect complementarity with 5-30 nucleotides of the first end of the RNA. In some embodiments, the oligonucleotide splint has perfect complementarity with 5-25 nucleotides of the first end of the RNA. In some embodiments, the oligonucleotide splint has perfect complementarity with 5-20 nucleotides of the first end of the RNA. In some embodiments, the oligonucleotide splint has perfect complementarity with 5-15 nucleotides of the first end of the RNA. In some embodiments, the oligonucleotide splint has perfect complementarity with 5-10 nucleotides of the first end of the RNA. In some embodiments, the RNA is messenger RNA. In some embodiments, the RNA is guide RNA.

In some embodiments, the oligonucleotide splint has perfect complementarity with 5-50 nucleotides of the first end of the guide RNA. In some embodiments, the oligonucleotide splint has perfect complementarity with 5-45 nucleotides of the first end of the guide RNA. In some embodiments, the oligonucleotide splint has perfect complementarity with 5-40 nucleotides of the first end of the guide RNA. In some embodiments, the oligonucleotide splint has perfect complementarity with 5-35 nucleotides of the first end of the guide RNA. In some embodiments, the oligonucleotide splint has perfect complementarity with 5-30 nucleotides of the first end of the guide RNA. In some embodiments, the oligonucleotide splint has perfect complementarity with 5-25 nucleotides of the first end of the guide RNA. In some embodiments, the oligonucleotide splint has perfect complementarity with 5-20 nucleotides of the first end of the guide RNA. In some embodiments, the oligonucleotide splint has perfect complementarity with 5-15 nucleotides of the first end of the guide RNA. In some embodiments, the oligonucleotide splint has perfect complementarity with 5-10 nucleotides of the first end of the guide RNA.

In some embodiments, the oligonucleotide splint has perfect complementarity with 100% of the RNA. In some embodiments, the oligonucleotide splint has perfect complementarity with 95% of the RNA. In some embodiments, the oligonucleotide splint has perfect complementarity with 90% of the RNA. In some embodiments, the oligonucleotide splint has perfect complementarity with 85% of the RNA. In some embodiments, the oligonucleotide splint has perfect complementarity with 80% of the RNA. In some embodiments, the oligonucleotide splint has perfect complementarity with 75% of the RNA. In some embodiments, the oligonucleotide splint has perfect complementarity with 70% of the RNA. In some embodiments, the oligonucleotide splint has perfect complementarity with 65% of the RNA. In some embodiments, the oligonucleotide splint has perfect complementarity with 60% of the RNA. In some embodiments, the oligonucleotide splint has perfect complementarity with 55% of the RNA. In some embodiments, the oligonucleotide splint has perfect complementarity with 50% of the RNA. In some embodiments, the oligonucleotide splint has perfect complementarity with 45% of the RNA. In some embodiments, the oligonucleotide splint has perfect complementarity with 40% of the RNA. In some embodiments, the oligonucleotide splint has perfect complementarity with 35% of the RNA. In some embodiments, the oligonucleotide splint has perfect complementarity with 30% of the RNA. In some embodiments, the oligonucleotide splint has perfect complementarity with 25% of the RNA. In some embodiments, the oligonucleotide splint has perfect complementarity with 20% of the RNA. In some embodiments, the oligonucleotide splint has perfect complementarity with 15% of the RNA. In some embodiments, the oligonucleotide splint has perfect complementarity with 10% of the RNA. In some embodiments, the oligonucleotide splint has perfect complementarity with 5% of the RNA. In some embodiments, the oligonucleotide splint has perfect complementarity with 2% of the RNA. In some embodiments, the oligonucleotide splint has perfect complementarity with 1% of the RNA. In some embodiments, the RNA is messenger RNA. In some embodiments, the RNA is guide RNA.

In some embodiments, the oligonucleotide splint has perfect complementarity with 100% of the guide RNA. In some embodiments, the oligonucleotide splint has perfect complementarity with 95% of the guide RNA. In some embodiments, the oligonucleotide splint has perfect complementarity with 90% of the guide RNA. In some embodiments, the oligonucleotide splint has perfect complementarity with 85% of the guide RNA. In some embodiments, the oligonucleotide splint has perfect complementarity with 80% of the guide RNA. In some embodiments, the oligonucleotide splint has perfect complementarity with 75% of the guide RNA. In some embodiments, the oligonucleotide splint has perfect complementarity with 70% of the guide RNA. In some embodiments, the oligonucleotide splint has perfect complementarity with 65% of the guide RNA. In some embodiments, the oligonucleotide splint has perfect complementarity with 60% of the guide RNA. In some embodiments, the oligonucleotide splint has perfect complementarity with 55% of the guide RNA. In some embodiments, the oligonucleotide splint has perfect complementarity with 50% of the guide RNA. In some embodiments, the oligonucleotide splint has perfect complementarity with 45% of the guide RNA. In some embodiments, the oligonucleotide splint has perfect complementarity with 40% of the guide RNA. In some embodiments, the oligonucleotide splint has perfect complementarity with 35% of the guide RNA. In some embodiments, the oligonucleotide splint has perfect complementarity with 30% of the guide RNA. In some embodiments, the oligonucleotide splint has perfect complementarity with 25% of the guide RNA. In some embodiments, the oligonucleotide splint has perfect complementarity with 20% of the guide RNA. In some embodiments, the oligonucleotide splint has perfect complementarity with 15% of the guide RNA. In some embodiments, the oligonucleotide splint has perfect complementarity with 10% of the guide RNA. In some embodiments, the oligonucleotide splint has perfect complementarity with 5% of the guide RNA. In some embodiments, the oligonucleotide splint has perfect complementarity with 2% of the guide RNA. In some embodiments, the oligonucleotide splint has perfect complementarity with 1% of the guide RNA.

In some embodiments, the method comprises a ligating enzyme wherein the ligating enzyme is a nick or template-mediated ligase. In some embodiments, wherein the nick or template-mediated ligase is T4 RNA ligase 2.

In some embodiments, the RNA is chemically synthesized. In some embodiments, the guide RNA is chemically synthesized. In some embodiments, the messenger RNA is chemically synthesized.

In some embodiments, the RNA is enzymatically synthesized. In some embodiments, wherein the RNA is synthesized enzymatically using T7 RNA polymerase. In some embodiments, the RNA is synthesized enzymatically using T3 RNA polymerase. In some embodiments, the RNA is synthesized enzymatically using SP6 RNA polymerase.

In some embodiments, the guide RNA is enzymatically synthesized. In some embodiments, wherein the guide RNA is synthesized enzymatically using T7 RNA polymerase. In some embodiments, the guide RNA is synthesized enzymatically using T3 RNA polymerase. In some embodiments, the guide RNA is synthesized enzymatically using SP6 RNA polymerase.

In some embodiments, the messenger RNA is enzymatically synthesized. In some embodiments, the messenger RNA is synthesized enzymatically using T7 RNA polymerase. In some embodiments, the messenger RNA is synthesized enzymatically using T3 RNA polymerase. In some embodiments, the messenger RNA is synthesized enzymatically using SP6 RNA polymerase.

In some embodiments, the enzymatically synthesized RNA comprises a 5′ triphosphate. In some embodiments, mRNA comprising a terminal 5′ triphosphate is digested to mRNA comprising a terminal 5′ monophosphate. In some embodiments, RNA 5′ pyrophosphohydrolase (RppH) enzyme catalyzes the conversion of mRNA comprising a terminal 5′ triphosphate to mRNA comprising a terminal 5′ monophosphate. In some embodiments, mRNA comprising a terminal 5′ monophosphate is generated by the addition of guanosine monophosphate (GMP) to guanosine triphosphate (GTP) during in vitro transcription for synthesis of mRNA.

In some embodiments, the mRNA comprising terminal 5′ monophosphate is subsequently circularized by a ligase. In some embodiments, the ligase is T4 RNA ligase 1. In some embodiments, the ligase is T4 RNA ligase 2.

In some embodiments, the guide RNA and/or messenger RNA further comprises a linker sequence. In some embodiments, the linker sequence is an RNA sequence.

In some embodiments, the linker sequence is positioned at the 5′ end and/or 3′ end of the guide RNA or messenger RNA sequence.

In some embodiments, the linker sequence is positioned between the first end of the guide RNA and the second end of the guide RNA. In some embodiments, the linker sequence is positioned between the first end of the messenger RNA and the second end of the messenger RNA.

In some embodiments, the linker comprises about 1-50 nucleotides. In some embodiments, the linker comprises about 1-45 nucleotides. In some embodiments, the linker comprises about 1-40 nucleotides. In some embodiments, the linker comprises about 1-35 nucleotides. In some embodiments, the linker comprises about 1-30 nucleotides. In some embodiments, the linker comprises about 1-25 nucleotides. In some embodiments, the linker comprises about 1-20 nucleotides. In some embodiments, the linker comprises about 1-15 nucleotides. In some embodiments, the linker comprises about 1-10 nucleotides. In some embodiments, the linker comprises about 1-5 nucleotides.

In some embodiments, the linker comprises about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, or about 50 nucleotides.

In some embodiments, the guide RNA or messenger RNA does not comprise a linker. In some embodiments, the linker comprises about one nucleotide. In some embodiments, the linker comprises about 2 nucleotides. In some embodiments, the linker comprises about 3 nucleotides. In some embodiments, the linker comprises about 4 nucleotides. In some embodiments, the linker comprises about 5 nucleotides. In some embodiments, the linker comprises about 6 nucleotides. In some embodiments, the linker comprises about 7 nucleotides. In some embodiments, the linker comprises about 8 nucleotides. In some embodiments, the linker comprises about 9 nucleotides. In some embodiments, the linker comprises about 10 nucleotides. In some embodiments, the linker comprises about 11 nucleotides. In some embodiments, the linker comprises about 12 nucleotides. In some embodiments, the linker comprises about 13 nucleotides. In some embodiments, the linker comprises about 14 nucleotides. In some embodiments, the linker comprises about 15 nucleotides. In some embodiments, the linker comprises about 16 nucleotides. In some embodiments, the linker comprises about 17 nucleotides. In some embodiments, the linker comprises about 18 nucleotides. In some embodiments, the linker comprises about 19 nucleotides. In some embodiments, the linker comprises about 20 nucleotides. In some embodiments, the linker comprises about 21 nucleotides. In some embodiments, the linker comprises about 22 nucleotides. In some embodiments, the linker comprises about 23 nucleotides. In some embodiments, the linker comprises about 24 nucleotides. In some embodiments, the linker comprises about 25 nucleotides.

In some embodiments, the linker comprises between about 50-100 nucleotides. In some embodiments, the linker comprises between about 100-150 nucleotides. In some embodiments, the linker comprises between about 150-200 nucleotides. In some embodiments, the linker comprises between about 200-500 nucleotides.

In some embodiments, the guide RNA further comprises a direct repeat sequence found in natural CRISPR systems.

In one aspect, provided herein is a method of making circular guide RNA (cgRNA) comprising modifying the ends of a linear guide RNA to create a first 5′ hydroxyl end and a second 2′-3′ cyclic phosphate end; and ligating the first end and the second end with an RNA ligase thereby generating a cgRNA. In another aspect, provided herein is a method of making circular messenger RNA comprising modifying the ends of a linear messenger RNA to create a first 5′ hydroxyl end and a second 2′-3′ cyclic phosphate end; and ligating the first end and the second end with an RNA ligase thereby generating a circular messenger RNA.

In some aspects, a method of producing a synthetic circular guide RNA (gRNA) or messenger RNA is provided comprising: providing two or more RNA fragments, with one comprising a 5′ monophosphate; providing an oligonucleotide that has partial complementarity to the two or more RNA fragments, wherein the complementarity of the oligonucleotide allows for base pairing with the two or more RNA fragments; and providing a ligase to catalyze ligation between the two or more RNA fragments, thus producing the synthetic circular guide RNA or circular messenger RNA. In some embodiments, the two or more RNA fragments are ligated at an overhang, blunt end, or at a bulge. In some embodiments, a first RNA comprises a 5′ monophosphate. In some embodiments, a second RNA comprises a blocked 3′ end. In some embodiments, the gRNA or messenger RNA comprises an adenosine triphosphate at the 5′ terminus.

In some embodiments, the ligating occurs in the absence of a template between the first end and the second end of the RNA. In some embodiments, the ligating occurs in the absence of an oligonucleotide splint between the first end and the second end of the RNA.

In some embodiments, the ligating occurs in the absence of a template between the first end and the second end of the guide RNA. In some embodiments, the ligating occurs in the absence of an oligonucleotide splint between the first end and the second end of the guide RNA.

In some embodiments, the ligating occurs in the absence of a template between the first end and the second end of the messenger RNA. In some embodiments, the ligating occurs in the absence of an oligonucleotide splint between the first end and the second end of the messenger RNA.

In some embodiments, the modifying the ends to create a first 5′ hydroxyl end and a second 2′-3′ cyclic phosphate end is performed by a ribozyme. In some embodiments, the modifying the ends to create a first 5′ hydroxyl end and a second 2′-3′ cyclic phosphate end is performed by a self-splicing ribozyme. In some embodiments, the modifying the ends to create a first 5′ hydroxyl end and a second 2′-3′ cyclic phosphate end is performed by introns. In some embodiments, the modifying the ends to create a first 5′ hydroxyl end and a second 2′-3′ cyclic phosphate end is performed by a twister ribozyme.

In some embodiments, the RNA ligase ligates an RNA comprising 2′,3′-cyclic phosphate and 5′-OH. In some embodiments, the RNA ligase is RtcB ligase.

In some embodiments, the guide RNA further comprises a linker sequence.

In some embodiments, the linker sequence is between 40 nucleotides and 250 nucleotides. In some embodiments, the linker comprises about 40-250 nucleotides. In some embodiments, the linker comprises about 40-200 nucleotides. In some embodiments, the linker comprises about 40-150 nucleotides. In some embodiments, the linker comprises about 40-100 nucleotides. In some embodiments, the linker comprises about 40-80 nucleotides. In some embodiments, the linker comprises about 40-70 nucleotides. In some embodiments, the linker comprises about 40-60 nucleotides. In some embodiments, the linker comprises about 40-50 nucleotides.

In some embodiments, the linker comprises about 100-250 nucleotides. In some embodiments, the linker comprises about 100-200 nucleotides. In some embodiments, the linker comprises about 100-150 nucleotides. In some embodiments, the linker comprises about 150-250 nucleotides. In some embodiments, the linker comprises about 150-200 nucleotides. In some embodiments, the linker comprises about 200-250 nucleotides.

In some embodiments, the linker sequence is a linear linker sequence. In some embodiments, the linker sequence is a non-linear sequence. In some embodiments, the linker sequence comprises RNA secondary structures.

In some embodiments, the linker sequence is placed at the 3′ end and/or the 5′ end of the RNA sequence. In some embodiments, the linker sequence is placed at the 3′ end and/or the 5′ end of the guide RNA sequence. In some embodiments, the linker sequence is placed at the 3′ end and/or the 5′ end of the messenger RNA sequence.

In some embodiments, the 3′ end of the RNA has a linker that is about 1-20 nucleotides, about 5-50 nucleotides, about 5-100 nucleotides, about 5-200 nucleotides, or about 5-250 nucleotides longer than a linker at the 5′ end of the RNA.

In some embodiments, the 3′ end of the guide RNA has a linker that is about 1-20 nucleotides, about 5-50 nucleotides, about 5-100 nucleotides, about 5-200 nucleotides, or about 5-250 nucleotides longer than a linker at the 5′ end of the guide RNA.

In some embodiments, the 3′ end of the messenger RNA has a linker that is about 1-30 nucleotides, about 10-50 nucleotides, about 25-100 nucleotides, about 30-200 nucleotides, or about 5-250 nucleotides longer than a linker at the 5′ end of the messenger RNA.

In some embodiments, the 5′ end of the RNA has a linker that is between 1-20 nucleotides, 5-50 nucleotides, 5-100 nucleotides longer than a linker at the 3′ end of the RNA.

In some embodiments, the 5′ end of the guide RNA has a linker that is between 1-20 nucleotides, 5-50 nucleotides, 5-100 nucleotides longer than a linker at the 3′ end of the guide RNA.

In some embodiments, the 5′ end of the messenger RNA has a linker that is between 1-20 nucleotides, 5-50 nucleotides, 5-100 nucleotides longer than a linker at the 3′ end of the messenger RNA.

In some embodiments, the 3′ end of the guide RNA has a linker that is about one nucleotide longer than a linker at the 5′ end of the guide RNA. In some embodiments, wherein the 3′ end of the guide RNA has a linker that is about two nucleotides longer than a linker at the 5′ end of the guide RNA. In some embodiments, wherein the 3′ end of the guide RNA has a linker that is about 1-50 nucleotides longer than a linker at the 5′ end of the guide RNA. In some embodiments, wherein the 3′ end of the guide RNA has a linker that is about 1-45 nucleotides longer than a linker at the 5′ end of the guide RNA. In some embodiments, wherein the 3′ end of the guide RNA has a linker that is about 1-40 nucleotides longer than a linker at the 5′ end of the guide RNA. In some embodiments, wherein the 3′ end of the guide RNA has a linker that is about 1-35 nucleotides longer than a linker at the 5′ end of the guide RNA. In some embodiments, wherein the 3′ end of the guide RNA has a linker that is about 1-30 nucleotides longer than a linker at the 5′ end of the guide RNA. In some embodiments, wherein the 3′ end of the guide RNA has a linker that is about 1-25 nucleotides longer than a linker at the 5′ end of the guide RNA. In some embodiments, wherein the 3′ end of the guide RNA has a linker that is about 1-20 nucleotides longer than a linker at the 5′ end of the guide RNA. In some embodiments, wherein the 3′ end of the guide RNA has a linker that is about 1-15 nucleotides longer than a linker at the 5′ end of the guide RNA. In some embodiments, wherein the 3′ end of the guide RNA has a linker that is about 1-10 nucleotides longer than a linker at the 5′ end of the guide RNA. In some embodiments, wherein the 3′ end of the guide RNA has a linker that is about 1-5 nucleotides longer than a linker at the 5′ end of the guide RNA.

In some embodiments, the 3′ end of the messenger RNA has a linker that is about one nucleotide longer than a linker at the 5′ end of the messenger RNA. In some embodiments, wherein the 3′ end of the messenger RNA has a linker that is about two nucleotides longer than a linker at the 5′ end of the messenger RNA. In some embodiments, wherein the 3′ end of the messenger RNA has a linker that is about 1-50 nucleotides longer than a linker at the 5′ end of the messenger RNA. In some embodiments, wherein the 3′ end of the messenger RNA has a linker that is about 1-45 nucleotides longer than a linker at the 5′ end of the messenger RNA. In some embodiments, wherein the 3′ end of the messenger RNA has a linker that is about 1-40 nucleotides longer than a linker at the 5′ end of the messenger RNA. In some embodiments, wherein the 3′ end of the messenger RNA has a linker that is about 1-35 nucleotides longer than a linker at the 5′ end of the messenger RNA. In some embodiments, wherein the 3′ end of the messenger RNA has a linker that is about 1-30 nucleotides longer than a linker at the 5′ end of the messenger RNA. In some embodiments, wherein the 3′ end of the messenger RNA has a linker that is about 1-25 nucleotides longer than a linker at the 5′ end of the messenger RNA. In some embodiments, wherein the 3′ end of the messenger RNA has a linker that is about 1-20 nucleotides longer than a linker at the 5′ end of the messenger RNA. In some embodiments, wherein the 3′ end of the messenger RNA has a linker that is about 1-15 nucleotides longer than a linker at the 5′ end of the messenger RNA. In some embodiments, wherein the 3′ end of the messenger RNA has a linker that is about 1-10 nucleotides longer than a linker at the 5′ end of the messenger RNA. In some embodiments, wherein the 3′ end of the messenger RNA has a linker that is about 1-5 nucleotides longer than a linker at the 5′ end of the messenger RNA.

In some embodiments, the 5′ end of the guide RNA has a linker that is about one nucleotide longer than a linker at the 3′ end of the guide RNA. In some embodiments, the 5′ end of the guide RNA has a linker that is about two nucleotides longer than a linker at the 3′ end of the guide RNA. In some embodiments, the 5′ end of the guide RNA has a linker that is about 1-50 nucleotides longer than a linker at the 3′ end of the guide RNA. In some embodiments, the 5′ end of the guide RNA has a linker that is about 1-45 nucleotides longer than a linker at the 3′ end of the guide RNA. In some embodiments, the 5′ end of the guide RNA has a linker that is about 1-40 nucleotides longer than a linker at the 3′ end of the guide RNA. In some embodiments, the 5′ end of the guide RNA has a linker that is about 1-35 nucleotides longer than a linker at the 3′ end of the guide RNA. In some embodiments, the 5′ end of the guide RNA has a linker that is about 1-30 nucleotides longer than a linker at the 3′ end of the guide RNA. In some embodiments, the 5′ end of the guide RNA has a linker that is about 1-25 nucleotides longer than a linker at the 3′ end of the guide RNA. In some embodiments, the 5′ end of the guide RNA has a linker that is about 1-20 nucleotides longer than a linker at the 3′ end of the guide RNA. In some embodiments, the 5′ end of the guide RNA has a linker that is about 1-15 nucleotides longer than a linker at the 3′ end of the guide RNA. In some embodiments, the 5′ end of the guide RNA has a linker that is about 1-10 nucleotides longer than a linker at the 3′ end of the guide RNA. In some embodiments, the 5′ end of the guide RNA has a linker that is about 1-5 nucleotides longer than a linker at the 3′ end of the guide RNA.

In some embodiments, the 5′ end of the messenger RNA has a linker that is about one nucleotide longer than a linker at the 3′ end of the messenger RNA. In some embodiments, the 5′ end of the messenger RNA has a linker that is about two nucleotides longer than a linker at the 3′ end of the messenger RNA. In some embodiments, the 5′ end of the messenger RNA has a linker that is about 1-50 nucleotides longer than a linker at the 3′ end of the messenger RNA. In some embodiments, the 5′ end of the messenger RNA has a linker that is about 1-45 nucleotides longer than a linker at the 3′ end of the messenger RNA. In some embodiments, the 5′ end of the messenger RNA has a linker that is about 1-40 nucleotides longer than a linker at the 3′ end of the messenger RNA. In some embodiments, the 5′ end of the messenger RNA has a linker that is about 1-35 nucleotides longer than a linker at the 3′ end of the messenger RNA. In some embodiments, the 5′ end of the messenger RNA has a linker that is about 1-30 nucleotides longer than a linker at the 3′ end of the messenger RNA. In some embodiments, the 5′ end of the messenger RNA has a linker that is about 1-25 nucleotides longer than a linker at the 3′ end of the messenger RNA. In some embodiments, the 5′ end of the messenger RNA has a linker that is about 1-20 nucleotides longer than a linker at the 3′ end of the messenger RNA. In some embodiments, the 5′ end of the messenger RNA has a linker that is about 1-15 nucleotides longer than a linker at the 3′ end of the messenger RNA. In some embodiments, the 5′ end of the messenger RNA has a linker that is about 1-10 nucleotides longer than a linker at the 3′ end of the messenger RNA. In some embodiments, the 5′ end of the messenger RNA has a linker that is about 1-5 nucleotides longer than a linker at the 3′ end of the messenger RNA.

In some embodiments, the linker comprises about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, or about 50 nucleotides. Accordingly, in some embodiments, the linker comprises about 5 nucleotides. Accordingly, in some embodiments, the linker comprises about 10 nucleotides. Accordingly, in some embodiments, the linker comprises about 15 nucleotides. Accordingly, in some embodiments, the linker comprises about 20 nucleotides. Accordingly, in some embodiments, the linker comprises about 25 nucleotides. Accordingly, in some embodiments, the linker comprises about 30 nucleotides. Accordingly, in some embodiments, the linker comprises about 35 nucleotides. Accordingly, in some embodiments, the linker comprises about 40 nucleotides. Accordingly, in some embodiments, the linker comprises about 45 nucleotides. Accordingly, in some embodiments, the linker comprises about 50 nucleotides.

In some embodiments, the circular RNA is non-coding. In some embodiments, the circular RNA encodes a protein. In some embodiments, the circular RNA is messenger RNA. In some embodiments, the circular messenger RNA further comprises a non-coding element. In some embodiments, the circular messenger RNA comprises an internal ribosome entry site (IRES) element. In some embodiments, the circular messenger RNA comprises one or more untranslated regions, i.e. a 5′ UTR and/or a 3′ UTR. In some embodiments, the circular messenger RNA comprises a 5′ UTR. In some embodiments, the circular messenger RNA comprises a 3′ UTR.

In some embodiments, the circular RNA codes for a protein. In some embodiments, the circular RNA codes for an enzyme. In some embodiments, the circular messenger RNA encodes one or more components of CRISPR-Cas systems. In some embodiments, the circular messenger RNA encodes a Cas enzyme.

In some embodiments, the circular messenger RNA encodes a Cas enzyme selected from the group consisting of Cas9, Cas13 or Cas12 (e.g. Cas12a, Cas12b, Cas12c, Cas12d, Cas12e, Cas12f, Cas12g, Cas12h, Cas12i, Cas12j, Cas12k). In some embodiments, the circular messenger RNA encodes a Cas9 enzyme. In some embodiments, the circular messenger RNA encodes a Cas13 enzyme. In some embodiments, the circular messenger RNA encodes a Cas12 enzyme. In some embodiments, the circular messenger RNA encodes a Cas12a, Cas12b, Cas12c, Cas12d, Cas12e, Cas12f, Cas12g, Cas12h, Cas12i, Cas12j, or Cas12k enzyme. In some embodiments, the circular messenger RNA encodes a Cas12a enzyme. In some embodiments, the circular messenger RNA encodes a Cas12b enzyme. In some embodiments, the circular messenger RNA encodes a Cas12c enzyme. In some embodiments, the circular messenger RNA encodes a Cas12d enzyme. In some embodiments, the circular messenger RNA encodes a Cas12e enzyme. In some embodiments, the circular messenger RNA encodes a Cas12f enzyme. In some embodiments, the circular messenger RNA encodes a Cas12g enzyme. In some embodiments, the circular messenger RNA encodes a Cas12h enzyme. In some embodiments, the circular messenger RNA encodes a Cas12i enzyme. In some embodiments, the circular messenger RNA encodes a Cas12j enzyme.

In some embodiments, the circular messenger RNA encodes a deaminase enzyme. In some embodiments, the circular messenger RNA encodes a cytosine deaminase or a cytidine deaminase. In some embodiments, the circular messenger RNA encodes an adenosine deaminase or an adenine deaminase. In some embodiments, the circular messenger RNA encodes a base editor. In some embodiments, the circular messenger RNA is circular guide RNA.

In some embodiments, the guide RNA comprises a clustered regularly interspersed short palindromic repeats (CRISPR) RNA (crRNA). In some embodiments, the guide RNA further comprises a trans-activating RNA (tracrRNA).

In some embodiments, the crRNA is modified. In some embodiments, the tracrRNA is modified. In some embodiments, the crRNA and/or comprise chemically modified nucleotides. In some embodiments, the tracrRNA comprises additional sequences that maintain folding. In some embodiments, the linker comprises chemically modified nucleotides.

In some embodiments, the modifications to the crRNA, tracrRNA, and/or linker comprises one or more of 1) chemical modifications; 2) any nucleotide substitutions that preserve secondary structure; 3) alterations of the GC content; 4) addition of sequence to maintain predicted folding of tracrRNA.

In some embodiments provided herein is a method, wherein the cgRNA is an extended guide RNA, or a Cas9 guide RNA, or a Cas13 guide RNA, or a Cas12 guide RNA such as Cas12a guide RNA, Cas12b guide RNA, Cas12c guide RNA, Cas12d, guide RNA, Cas12e guide RNA, Cas12f guide RNA, Cas12g guide RNA, Cas12h guide RNA, Cas12i guide RNA, Cas12j guide RNA, Cas12k guide RNA. Accordingly, in some embodiments, the cgRNA is an extended guide RNA. In some embodiments, the cgRNA is a Cas9 guide RNA. In some embodiments, the cgRNA is a Cas13 guide RNA. In some embodiments, the cgRNA is a Cas12 guide RNA. In some embodiments, the cgRNA is a Cas12a guide RNA. In some embodiments, the cgRNA is a Cas12b guide RNA. In some embodiments, the cgRNA is a Cas12c guide RNA. In some embodiments, the cgRNA is a Cas12d guide RNA. In some embodiments, the cgRNA is a Cas12e guide RNA. In some embodiments, the cgRNA is a Cas12f guide RNA. In some embodiments, the cgRNA is a Cas12g guide RNA. In some embodiments, the cgRNA is a Cas12h guide RNA. In some embodiments, the cgRNA is a Cas12i guide RNA. In some embodiments, the cgRNA is a Cas12j guide RNA. In some embodiments, the cgRNA is a Cas12k guide RNA.

In some embodiments, the cgRNA comprises one or more of the following: a spacer, a lower stem, a bulge, an upper stem, a nexus and a hairpin.

In some embodiments, the stem loop comprises GC base pairs.

In some embodiments provided herein is a method, wherein the cgRNA is produced at a yield of about 50%, 55%, 60%, 65%, 70%, 75%, 80%, 85%, 90%, 95%, 99% or more. Accordingly, in some embodiments, the cgRNA is produced at a yield of about 50%. In some embodiments, the cgRNA is produced at a yield of about 55%. In some embodiments, the cgRNA is produced at a yield of about 60%. In some embodiments, the cgRNA is produced at a yield of about 65%. In some embodiments, the cgRNA is produced at a yield of about 70%. In some embodiments, the cgRNA is produced at a yield of about 75%. In some embodiments, the cgRNA is produced at a yield of about 80%. In some embodiments, the cgRNA is produced at a yield of about 85%. In some embodiments, the cgRNA is produced at a yield of about 90%. In some embodiments, the cgRNA is produced at a yield of about 95%. In some embodiments, the cgRNA is produced at a yield of more than 99%.

In some embodiments provided herein is a method, wherein the circular RNA is produced at a yield of about 50%, 55%, 60%, 65%, 70%, 75%, 80%, 85%, 90%, 95%, 99% or more. Accordingly, in some embodiments, the circular RNA is produced at a yield of about 50%. In some embodiments, the circular RNA is produced at a yield of about 55%. In some embodiments, the circular RNA is produced at a yield of about 60%. In some embodiments, the circular RNA is produced at a yield of about 65%. In some embodiments, the circular RNA is produced at a yield of about 70%. In some embodiments, the circular RNA is produced at a yield of about 75%. In some embodiments, the circular RNA is produced at a yield of about 80%. In some embodiments, the circular RNA is produced at a yield of about 85%. In some embodiments, the circular RNA is produced at a yield of about 90%. In some embodiments, the circular RNA is produced at a yield of about 95%. In some embodiments, the circular RNA is produced at a yield of more than 99%. In some embodiments, the circular RNA is circular messenger RNA. In some embodiments, the circular RNA is circular guide RNA.

In some embodiments, the cgRNA is produced at 50%, 55%, 60%, 65%, 70%, 75%, 80%, 85%, 90%, 95%, 99% or more improvement in yield as compared to conventional synthetic methods. Accordingly, in some embodiments, the cgRNA is produced at 50% improvement in yield as compared to conventional synthetic methods. In some embodiments, the cgRNA is produced at 55% improvement in yield as compared to conventional synthetic methods. In some embodiments, the cgRNA is produced at 60% improvement in yield as compared to conventional synthetic methods. In some embodiments, the cgRNA is produced at 55% improvement in yield as compared to conventional synthetic methods. In some embodiments, the cgRNA is produced at 60% improvement in yield as compared to conventional synthetic methods. In some embodiments, the cgRNA is produced at 65% improvement in yield as compared to conventional synthetic methods. In some embodiments, the cgRNA is produced at 70% improvement in yield as compared to conventional synthetic methods. In some embodiments, the cgRNA is produced at 75% improvement in yield as compared to conventional synthetic methods. In some embodiments, the cgRNA is produced at 80% improvement in yield as compared to conventional synthetic methods. In some embodiments, the cgRNA is produced at 85% improvement in yield as compared to conventional synthetic methods. In some embodiments, the cgRNA is produced at 90% improvement in yield as compared to conventional synthetic methods. In some embodiments, the cgRNA is produced at 95% improvement in yield as compared to conventional synthetic methods. In some embodiments, the cgRNA is produced at 99% improvement in yield as compared to conventional synthetic methods. In some embodiments, the cgRNA is produced at more than 99% improvement in yield as compared to conventional synthetic methods.

In some embodiments, the circular RNA is produced at 50%, 55%, 60%, 65%, 70%, 75%, 80%, 85%, 90%, 95%, 99% or more improvement in yield as compared to conventional synthetic methods. Accordingly, in some embodiments, the circular RNA is produced at 50% improvement in yield as compared to conventional synthetic methods. In some embodiments, the circular RNA is produced at 55% improvement in yield as compared to conventional synthetic methods. In some embodiments, the circular RNA is produced at 60% improvement in yield as compared to conventional synthetic methods. In some embodiments, the circular RNA is produced at 55% improvement in yield as compared to conventional synthetic methods. In some embodiments, the circular RNA is produced at 60% improvement in yield as compared to conventional synthetic methods. In some embodiments, the circular RNA is produced at 65% improvement in yield as compared to conventional synthetic methods. In some embodiments, the circular RNA is produced at 70% improvement in yield as compared to conventional synthetic methods. In some embodiments, the circular RNA is produced at 75% improvement in yield as compared to conventional synthetic methods. In some embodiments, the circular RNA is produced at 80% improvement in yield as compared to conventional synthetic methods. In some embodiments, the circular RNA is produced at 85% improvement in yield as compared to conventional synthetic methods. In some embodiments, the circular RNA is produced at 90% improvement in yield as compared to conventional synthetic methods. In some embodiments, the circular RNA is produced at 95% improvement in yield as compared to conventional synthetic methods. In some embodiments, the circular RNA is produced at 99% improvement in yield as compared to conventional synthetic methods. In some embodiments, the circular RNA is produced at more than 99% improvement in yield as compared to conventional synthetic methods.

In some embodiments, the cgRNA has a length of about 40 nucleotides, about 100 nucleotides, about 125 nucleotides, about 150 nucleotides, about 175 nucleotides, about 200 nucleotides, or greater than about 200 nucleotides. Accordingly, in some embodiments, the cgRNA has a length of about 40 nucleotides. In some embodiments, the cgRNA has a length of about 100 nucleotides. In some embodiments, the cgRNA has a length of about 125 nucleotides. In some embodiments, the cgRNA has a length of about 150 nucleotides. In some embodiments, the cgRNA has a length of about 175 nucleotides. In some embodiments, the cgRNA has a length of about 200 nucleotides. In some embodiments, the cgRNA has a length of greater than about 200 nucleotides.

In some embodiments, the circular RNA has a length of between about 200 to 1000 nucleotides. In some embodiments, the circular RNA has a length of about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides or greater than about 1000 nucleotides. Accordingly, in some embodiments, the circular RNA has a length of about 100 nucleotides. In some embodiments, the circular RNA has a length of about 200 nucleotides. In some embodiments, the circular RNA has a length of about 300 nucleotides. In some embodiments, the circular RNA has a length of about 400 nucleotides. In some embodiments, the circular RNA has a length of about 500 nucleotides. In some embodiments, the circular RNA has a length of about 600 nucleotides. In some embodiments, the circular RNA has a length of greater than about 700 nucleotides. In some embodiments, the circular RNA has a length of greater than about 800 nucleotides. In some embodiments, the circular RNA has a length of greater than about 900 nucleotides. In some embodiments, the circular RNA has a length of greater than about 1000 nucleotides. In some embodiments, the circular RNA is circular messenger RNA.

In some embodiments, the circular RNA has a length of between about 1000 to 10,000 nucleotides. In some embodiments, the circular RNA has a length of between about 3000 to 6000 nucleotides. In some embodiments, the circular RNA has a length of about 1000 nucleotides, about 1500 nucleotides, about 2000 nucleotides, about 2500 nucleotides, about 3000 nucleotides, about 3500 nucleotides, about 4000 nucleotides, about 4500 nucleotides, about 5000 nucleotides, about 5500 nucleotides, about 6000 nucleotides, about 6500 nucleotides, about 7000 nucleotides, about 7500 nucleotides, about 8000 nucleotides, about 8500 nucleotides, about 9000 nucleotides, about 9500 nucleotides, about 10,000 nucleotides or greater than about 10,000 nucleotides. Accordingly, in some embodiments, the circular RNA has a length of about 1000 nucleotides. In some embodiments, the circular RNA has a length of about 1500 nucleotides. In some embodiments, the circular RNA has a length of about 2000 nucleotides. In some embodiments, the circular RNA has a length of about 2500 nucleotides. In some embodiments, the circular RNA has a length of about 3000 nucleotides. In some embodiments, the circular RNA has a length of about 3500 nucleotides. In some embodiments, the circular RNA has a length of about 4000 nucleotides. In some embodiments, the circular RNA has a length of about 4500 nucleotides. In some embodiments, the circular RNA has a length of about 5000 nucleotides. In some embodiments, the circular RNA has a length of about 5500 nucleotides. In some embodiments, the circular RNA has a length of about 6000 nucleotides. In some embodiments, the circular RNA has a length of about 6500 nucleotides. In some embodiments, the circular RNA has a length of about 7000 nucleotides. In some embodiments, the circular RNA has a length of about 7500 nucleotides. In some embodiments, the circular RNA has a length of about 8000 nucleotides. In some embodiments, the circular RNA has a length of about 8500 nucleotides. In some embodiments, the circular RNA has a length of about 9000 nucleotides. In some embodiments, the circular RNA has a length of about 9500 nucleotides. In some embodiments, the circular RNA has a length of about 10,000 nucleotides. In some embodiments, the circular RNA has a length of greater than about 10,000 nucleotides. In some embodiments, the circular RNA is circular messenger RNA.

In some embodiments, the circular RNA has a length of between about 1 kb to about 10 kb. In some embodiments, the circular RNA has a length of between about 3 kb to about 6 kb. In some embodiments, the circular RNA has a length of greater than about 10 kb. In some embodiments, the circular RNA has a length of about 1 kb. In some embodiments, the circular RNA has a length of about 2 kb. In some embodiments, the circular RNA has a length of about 3 kb. In some embodiments, the circular RNA has a length of about 4 kb. In some embodiments, the circular RNA has a length of about 5 kb. In some embodiments, the circular RNA has a length of about 6 kb. In some embodiments, the circular RNA has a length of about 7 kb. In some embodiments, the circular RNA has a length of about 8 kb. In some embodiments, the circular RNA has a length of about 9 kb. In some embodiments, the circular RNA has a length of about 10 kb.

In some embodiments, the cgRNA length is Cas dependent. For example, in some embodiments, the cgRNA length for Cas12a is greater than 40 nucleotides. In some embodiments, the cgRNA length for Cas9 is greater than 123 nucleotides. In some embodiments, the cgRNA length for Cas9 is between 125-200 nucleotides. In some embodiments, the cgRNA length for Cas9 is between 125-250 nucleotides. In some embodiments, the cgRNA length for Cas9 is between 125-300 nucleotides. In some embodiments, the cgRNA length for Cas9 is between 125-350 nucleotides. In some embodiments, the cgRNA length for Cas9 is between 125-400 nucleotides. In some embodiments, the cgRNA length for Cas9 is between 125-450 nucleotides. In some embodiments, the cgRNA length for Cas9 is between 125-500 nucleotides.

In some embodiments, the circular RNA comprises one or more backbone modifications. In some embodiments, the cgRNA comprises one or more backbone modifications. In some embodiments, the circular messenger RNA comprises one or more backbone modifications.

In some embodiments, the one or more backbone modifications comprises a 2′ O-methyl or a phosphorothioate modification. Accordingly, in some embodiments, the one or more backbone modifications comprises a 2′ O-methyl modification. In some embodiments, the one or more backbone modifications comprises a phosphorothioate modification.

In some embodiments, the one or more backbone modifications is selected from 2′-O-methyl 3′-phosphorothioate, 2′O-methyl, 2′-ribo 3′-phosphorothioate, 2′-fluro, 2′-O-methoxyethyl morpholino (PMO), locked nucleic acid (LNA), deoxy, or 5′ phosphate modification. Accordingly, in some embodiments, the one or more backbone modifications comprises a 2′-O-methyl 3′-phosphorothioate modification. In some embodiments, the one or more backbone modifications comprises a 2′-O-methyl modification. In some embodiments, the one or more modifications comprises a 2′-ribo 3′-phosphorothioate modification. In some embodiments, the one or more modifications comprises a 2′-fluro modification. In some embodiments, the one or more modifications comprises a locked nucleic acid (LNA). In some embodiments, the one or more modifications comprises a 2′-O-methoxyethyl morpholino (PMO). In some embodiments, the one or more modifications comprises a deoxy modification. In some embodiments, the one or more modifications comprises a 5′ phosphate modification.

Various modified RNA bases are known in the art and include for example, 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′OMethyl modifications can also be used with the methods described herein. For example, the following RNA comprising one or more of the following 2′OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the RNA comprises one or more of the following modifications: phosphorothioates, 2′O-methyls, 2′ fluoro (2′F), DNA. In some embodiments, the RNA comprises 2′OMe modifications at the 3′ and 5′-ends. In some embodiments, the RNA comprises one or more of the following modifications: 2′-O-2-Methoxyethyl (MOE), locked nucleic acids, bridged nucleic acids, unlocked nucleic acids, peptide nucleic acids, morpholino nucleic acids. In some embodiments, the RNA comprises one or more of the following base modifications: 2,6-diaminopurine, 2-aminopurine, pseudouracil, N1-methyl-psuedouracil, 5′ methyl cytosine, N6-methyladenosine, 2′pyrimidinone (zebularine), thymine. Other modified bases include for example, 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Desoxy-r(A,C,G,U), 3′-O-Methyl-r(A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

In some embodiments, the circular RNA (e.g. cgRNA or circular messenger RNA) is produced at a quantity of less than or about 1 gram. In some embodiments, the circular RNA (e.g. cgRNA or circular messenger RNA) is produced at a quantity of about 100 μg, 200 μg, 300 μg, 400 μg, 500 μg, 600 μg, 700 μg, 800 μg, 900 μg, or 1,000 μg. In some embodiments, the circular RNA (e.g. cgRNA or circular messenger RNA) is produced at a quantity of about 100 μg. In some embodiments, the circular RNA (e.g. cgRNA or circular messenger RNA) is produced at a quantity of about 200 μg. In some embodiments, the circular RNA (e.g. cgRNA or circular messenger RNA) is produced at a quantity of about 300 μg. In some embodiments, the circular RNA (e.g. cgRNA or circular messenger RNA) is produced at a quantity of about 400 μg. In some embodiments, the circular RNA (e.g. cgRNA or circular messenger RNA) is produced at a quantity of about 500 μg. In some embodiments, the circular RNA (e.g. cgRNA or circular messenger RNA) is produced at a quantity of about 600 μg. In some embodiments, the circular RNA (e.g. cgRNA or circular messenger RNA) is produced at a quantity of about 700 μg. In some embodiments, the circular RNA (e.g. cgRNA or circular messenger RNA) is produced at a quantity of about 800 μg. In some embodiments, the circular RNA (e.g. cgRNA or circular messenger RNA) is produced at a quantity of about 900 μg. In some embodiments, the circular RNA (e.g. cgRNA or circular messenger RNA) is produced at a quantity of about 1,000 μg.

In some embodiments, the method produces circular RNA (e.g. cgRNA or circular messenger RNA) at a purity of about 50%, 60%, 70%, 80%, 90%, or more than 90%. Accordingly, in some embodiments, the method produces circular RNA (e.g. cgRNA or circular messenger RNA) at a purity of about 50%. In some embodiments, the method produces circular RNA (e.g. cgRNA or circular messenger RNA) at a purity of about 60%. In some embodiments, the method produces circular RNA (e.g. cgRNA or circular messenger RNA) at a purity of about 70%. In some embodiments, the method produces circular RNA (e.g. cgRNA or circular messenger RNA) at a purity of about 80%. In some embodiments, the method produces circular RNA (e.g. cgRNA or circular messenger RNA) at a purity of about 90%. In some embodiments, wherein the method produces circular RNA (e.g. cgRNA or circular messenger RNA) at a purity of about 91%, about 92%, about 93%, about 94%, about 95%, about 96%, about 97%, about 98%, about 99%, or more than 99%. In some embodiments, the method produces circular RNA (e.g. cgRNA or circular messenger RNA) at a purity of about 91%. In some embodiments, the method produces circular RNA (e.g. cgRNA or circular messenger RNA) at a purity of about 92%. In some embodiments, the method produces circular RNA (e.g. cgRNA or circular messenger RNA) at a purity of about 93%. In some embodiments, the method produces circular RNA (e.g. cgRNA or circular messenger RNA) at a purity of about 94%. In some embodiments, the method produces circular RNA (e.g. cgRNA or circular messenger RNA) at a purity of about 95%. In some embodiments, the method produces circular RNA (e.g. cgRNA or circular messenger RNA) at a purity of about 96%. In some embodiments, the method produces circular RNA (e.g. cgRNA or circular messenger RNA) at a purity of about 97%. In some embodiments, the method produces circular RNA (e.g. cgRNA or circular messenger RNA) at a purity of about 98%. In some embodiments, the method produces circular RNA (e.g. cgRNA or circular messenger RNA) at a purity of about 99%. In some embodiments, the method produces circular RNA (e.g. cgRNA or circular messenger RNA) at a purity of greater than about 99%.

In some embodiments, the cgRNA is suitable for use with CRISPR/Cas systems. In some embodiments, the cgRNA is suitable for use with CRISPR class 2 type II enzymes. In some embodiments, the cgRNA is suitable for use with CRISPR class 2 type V enzymes. In some embodiments, the cgRNA is suitable for use with CRISPR class 2 type VI enzymes. In some embodiments, wherein the cgRNA is suitable for use with Cas9, Cpf1, SaCas9, Cas12, Cas13, or modified versions thereof. Accordingly, in some embodiments, the cgRNA is suitable for use with Cas9, or modified versions thereof. In some embodiments, the cgRNA is suitable for use with Cpf1, or modified versions thereof. In some embodiments, the cgRNA is suitable for use with SaCas9, or modified versions thereof. In some embodiments, the cgRNA is suitable for use with Cas12, or modified versions thereof. In some embodiments, the cgRNA is suitable for use with Cas13, or modified versions thereof. In some embodiments, the cgRNA is in complex with the Cas enzyme.

In some embodiments, RNA sequences are included that will be cleaved by the endonuclease activity of some Cas e.g. Cas12a and Cas13 to linearize gRNA prior to or during assembly with Cas protein.

In some embodiments, the cgRNA provides increased stability and resistance to cellular exonucleases in comparison to linear guide RNA. In some embodiments, the cgRNA provides increased editing events in target cells using a CRISPR/Cas editing system.

In some embodiments, provided herein is a method for producing circular RNA that employs ligases, ribozymes and/or chemical ligation In some embodiments, provided herein is a method for producing synthetic circular guide RNA that employs ligases, ribozymes and/or chemical ligation. In some embodiments, provided herein is a method for producing synthetic circular messenger RNA that employs ligases, ribozymes and/or chemical ligation.

In some embodiments, provided herein is a composition comprising circular RNA produced by use of ligases, ribozymes and/or chemical modifications. In some embodiments, provided herein is a composition comprising cgRNA produced by use of ligases, ribozymes and/or chemical modifications. In some embodiments, provided herein is a composition comprising circular messenger RNA produced by use of ligases, ribozymes and/or chemical modifications.

In some embodiments, the cgRNA comprises one or more of the following: a spacer, a lower stem, a bulge, an upper stem, a nexus and a hairpin.

In some embodiments, the circular RNA has increased resistance to exonuclease in comparison to a linear RNA. In some embodiments, the cgRNA has increased resistance to exonuclease in comparison to a linear guide RNA. In some embodiments, the circular messenger RNA has increased resistance to exonuclease in comparison to a linear messenger RNA.

In some embodiments, the linear RNA has end modifications. In some embodiments, the linear guide RNA has end modifications. In some embodiments, the linear messenger RNA has end modifications.

In some aspects, a composition is provided comprising a circularized RNA. In some aspects, a composition is provided comprising a circularized guide RNA (cgRNA), the cgRNA comprising one or more of a spacer, a lower stem, a bulge, an upper stem, a nexus and a hairpin. In some other embodiments, a composition is provided comprising a circularized messenger RNA.

In some embodiments, the cgRNA is in a complex with a CRISPR class 2 type II enzyme. In some embodiments, the cgRNA is in a complex with a CRISPR class 2 type V enzyme. In some embodiments, the cgRNA is in a complex with a CRISPR class 2 type VI enzyme. In some embodiments, the cgRNA is in a complex with Cas9, Cpf1, SaCas9, Cas12, Cas13, or modified versions thereof.

In some embodiments, the circular RNA is produced by contacting a linear RNA with a ligating enzyme, wherein the contacting brings together a first end and a second end of the RNA, and wherein the ligating enzyme ligates the first and the second end of the RNA thus creating the circular RNA. In some embodiments, the cgRNA is produced by contacting a linear guide RNA with a ligating enzyme, wherein the contacting brings together a first end and a second end of the guide RNA, and wherein the ligating enzyme ligates the first and the second end of the guide RNA thus creating the cgRNA. In some embodiments, the circular messenger RNA is produced by contacting a linear messenger RNA with a ligating enzyme, wherein the contacting brings together a first end and a second end of the messenger RNA, and wherein the ligating enzyme ligates the first and the second end of the messenger RNA thus creating the circular messenger RNA.

In some embodiments, the circular RNA has increased resistance to exonuclease in comparison to a linear RNA. In some embodiments, the cgRNA has increased resistance to exonuclease in comparison to a linear guide RNA. In some embodiments, the circular messenger RNA has increased resistance to exonuclease in comparison to a linear messenger RNA. In some embodiments, the linear RNA has end modifications. In some embodiments, the linear guide RNA has end modifications. In some embodiments, the linear messenger RNA has end modifications.

In some aspects, a Cas protein complex is provided, the complex comprising a Cas nuclease and a circularized gRNA.

In some embodiments, the Cas nuclease is a CRISPR class 2 type II enzyme. In some embodiments, the Cas nuclease is a CRISPR class 2 type V enzyme. In some embodiments, the Cas nuclease is a CRISPR class 2 type VI In some embodiments, the Cas nuclease is selected from Cas9, Cpf1, SaCas9, Cas12, Cas13, or modified versions thereof.

In some embodiments, provided herein is a method for targeted transcription activation, targeted transcription repression, targeted epigenome modification, or targeted genome modification, the method comprising introducing into a eukaryotic cell: (a) a circular guide RNA (cgRNA), (b) at least one CRISPR/Cas protein or a nucleic acid encoding at least one CRISPR/Cas protein, wherein interactions between (a) and (b) and a target sequence in chromosomal DNA leads to targeted transcription activation, targeted transcription repression, targeted epigenome modification, or targeted genome modification.

In some embodiments, provided herein is a method for targeted RNA modification, the method comprising introducing into a eukaryotic cell: (a) a circular guide RNA (cgRNA) and (b) at least one CRISPR/Cas protein or a nucleic acid encoding the at least one CRISPR/Cas protein, wherein interactions between (a) and (b) and an RNA expressed by chromosomal DNA leads to a modification of the RNA expressed by the chromosomal DNA.

In some embodiments, the RNA expressed by the chromosomal DNA is a target messenger RNA (mRNA).

In some embodiments, provided herein is a method of making circular RNA, wherein the method comprises the steps of in vitro transcription in the presence of guanosine monophosphate, generating 5′ triphosphate, followed by circularization by self-splicing introns. In some embodiments, the self-splicing intron is Anabaena intron. In some embodiments, the self-splicing intron comprises SEQ ID NOs: 24 and 25. In some embodiments, the self-splicing intron is T4 intron. In some embodiments, the self-splicing intron comprises SEQ ID NOs: 26 and 27. In some embodiments, the T4 intron is modified. In some embodiments, the self-splicing intron comprises SEQ ID NOs: 28 and 29.

In some embodiments, provided herein is a kit comprising the composition comprising circular RNA produced by use of ligases, ribozymes or chemical modifications. In some embodiments, provided herein is a kit comprising the composition comprising cgRNA produced by use of ligases, ribozymes or chemical modifications. In some embodiments, provided herein is a kit comprising the composition comprising circular messenger RNA produced by use of ligases, ribozymes or chemical modifications.

In some embodiments, provided herein is a kit, wherein the kit further comprises one or more ligase, a linker sequence and one or more oligonucleotide splints.

In some embodiments, provided herein is a kit, wherein the ligase is a T4 RNA Ligase 2, T4 RNA Ligase 1 or RtcB ligase.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C: G-to-T: A base editors with higher efficiency and product purity” Science Advances 3: eaao4774 (2017), and Rees, H. A., et al., “Base editing: precision chemistry on the genome and transcriptome of living cells.” Nat Rev Genet. 2018 December; 19 (12): 770-788. doi: 10.1038/s41576-018-0059-1, the entire contents of which are hereby incorporated by reference.

Base Editing Activity: By “base editing activity” is meant acting to chemically alter a base within a polynucleotide (e.g., by deaminating the base). In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C. In another embodiment, the base editing activity is cytidine deaminase activity, e.g., converting target C·G to T·A and adenosine or adenine deaminase activity, e.g., converting A·T to G·C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: By “complementary” or “complementarity” is meant that a nucleic acid can form hydrogen bond(s) with another nucleic acid sequence by either traditional Watson-Crick or Hoogsteen base pairing. Complementary base pairing includes not only G-C and A-T base pairing, but also includes base pairing involving universal bases, such as inosine. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers. In some embodiments, the CRISPR array includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity and/or that binds to a target site on a nucleic acid specified by a RNA guide. In different embodiments, a CRISPR effector has endonuclease activity, nickase activity, exonuclease activity, transposase activity, and/or excision activity. In other embodiments, the CRISPR effector is nuclease inactive.

crRNA: The term “CRISPR RNA” or “crRNA,” as used herein, refers to a RNA molecule including a guide sequence used by a CRISPR effector to target a specific nucleic acid sequence. Typically, crRNAs contain a sequence that mediates target recognition and a sequence that forms a duplex with a tracrRNA. In some embodiments, the crRNA:tracrRNA duplex binds to a CRISPR effector.

Duplex: As used herein, “duplex” refers to a double helical structure formed by the interaction of two single stranded nucleic acids. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA, adenine (A) forms a base pair with thymine (T) in DNA, and adenine (A) forms a base pair with uracil (U) in RNA. Conditions under which base pairs can form include physiological or biologically relevant conditions (e.g., intracellular: pH 7.2, 140 mM potassium ion; extracellular pH 7.4, 145 mM sodium ion). Furthermore, duplexes are stabilized by stacking interactions between adjacent nucleotides. As used herein, a duplex may be established or maintained by base pairing or by stacking interactions. A duplex is formed by two complementary nucleic acid strands, which may be substantially complementary or fully complementary. Single-stranded nucleic acids that base pair over a number of bases are said to “hybridize.”

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Hybridize: By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

Hybridization occurs by hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Indel: As used herein, the term “indel” refers to insertion or deletion of bases in a nucleic acid sequence. It commonly results in mutations and is a common form of genetic variation.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Linker or Spacer: The linker or spacer is a nucleotide sequence that physically separates the terminal positions of the gRNA sequence to enable Cas binding and function of the gRNA. In some embodiments, the linker is RNA. In some embodiments, the linker is a chemical linker, for example, PEG9/18. In some embodiments, the linker is a DNA linker.

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. A PAM may be required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term “RNA guide” or “guide RNA” refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides. The term “RNA guide” or “guide RNA” also refers to circular guide RNA.

Single Strand Ligase: As used herein, the term “Single Strand Ligase” means a ligase that does not require an oligonucleotide splint or a template for its ligating activity.

Splint or Oligonucleotide Splint: The terms “splint” or “oligonucleotide splint” refers to refers to a single stranded RNA or DNA or other polymer that is capable of hybridizing with at least two, three or more single stranded RNA nucleotides. For example, the splint can refer to an oligonucleotide splint.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA,” “single guide RNA,” or “guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215 (3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, IRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1A is a general schematic which shows the synthesis of circular guide RNA (cgRNA) using T4 RNA ligase 2, which is a nick ligase requiring a splint.

FIG. 1B is a general schematic which shows the synthesis of circular guide RNA (cgRNA) using T4 RNA ligase 1, a single-strand ligase that does not require a template or splint.

FIG. 2A shows spectrophotometric readings of absorption at 260 nm. that depicts the amount of circular or cyclic gRNA synthesized by using T4 RNA ligase 2, relative to a control amount of an exemplary linear guide RNA in the absence of T4 RNA ligase 2.

FIG. 2B shows spectrophotometric readings of absorption at 260 nm. that depicts the amount of a circular or cyclic gRNA synthesized by using T4 RNA ligase 1, relative to a control amount of an exemplary linear guide RNA in the absence of T4 RNA ligase 1.

FIG. 3A shows spectrophotometric readings of absorption at 260 nm. that depicts the amount of circular or cyclic gRNA synthesized by using Cas9 gRNA and T4 RNA ligase 1, relative to a control amount of an exemplary gRNA in the absence of T4 RNA ligase 1.

FIG. 3B shows spectrophotometric readings of absorption at 260 nm. that depicts the amount of circular or cyclic gRNA synthesized by using Cas12 gRNA and T4 RNA ligase 1, relative to a control amount of an exemplary gRNA in the absence of T4 RNA ligase 1.

FIG. 3C shows spectrophotometric readings of absorption at 260 nm. that depicts the amount of circular or cyclic gRNA synthesized by using Cas13 gRNA and T4 RNA ligase 1, relative to a control amount of an exemplary gRNA in the absence of T4 RNA ligase 1.

FIG. 4 shows a schematic method of producing cyclic or circular gRNA. T4 RNA ligase 1 is used to circularize linear gRNAs. Production is scaled up and then the cgRNAs are analysed by HPLC and purified, for example, by anion exchange liquid chromatography.

FIG. 5A shows exemplary Cas12 cgRNA HPLC gradient analysis results after synthesis and scale-up to 500 ul reaction. FIG. 5B shows exemplary chromatograms after purification of Cas12 cgRNA.

FIG. 6A shows exemplary Cas13 cgRNA HPLC gradient analysis results after synthesis and scale-up to 500 ul reaction. FIG. 6B shows exemplary chromatograms after purification of Cas13 cgRNA.

FIG. 7A shows exemplary Cas9 cgRNA HPLC gradient analysis results after synthesis and scale-up to 500 ul reaction. FIG. 7B shows exemplary chromatograms after purification of Cas9 cgRNA.

FIG. 8 shows a graph depicting the percent indel frequency obtained with Cas12a using 150 ng mRNA and either 50 ng guide RNA (saturating gRNA concentration) or 5 ng guide RNA (subsaturating gRNA concentration). Comparative results are also provided for 42 nt guide RNAs, that are either circular or linear guide RNAs, and for linear guide RNAs with or without chemical end modifications. Results are provided for exemplary linker lengths of 10 nt used in combination with a 52 nt long cgRNA, linker of 20 nt in combination with a 62 nt cgRNA, linker of 30 nt in combination with 72 nt cgRNA, and direct repeats evaluated in combination with a 61 nt cgRNA. The percent indel frequency is a measure of gene editing efficiency.

FIG. 9 shows a graph depicting the percent indel frequency obtained with Cas12a using cgRNAs relative to linear gRNAs at exemplary gRNA concentrations of 500 μg, 5 ng, 50 ng and 100 ng. Two exemplary lengths of 42 nt and 61 nt were tested. The cgRNA and linear gRNA was used in combination with direct repeats. The percent indel frequency is a measure of gene editing efficiency.

FIG. 10 depicts a schematic method of generating circular guide RNAs using ribozymes. This method does not require a splint. Products are formed in three possible orientations: orientation 1, which comprises a linear linker at the 5′ end followed by target sequence, crRNA, broccoli marker, tracrRNA, and then a linear linker at 3′ end, and ligation occurs joining the linkers at 5′ OH and 2′3 cyclic phosphate ends producing cgRNA; orientation 2, which comprises a modified tracrRNA which helps proper folding and ligation occurs at the GAAA tetraloop connecting the tracrRNA at the 5′ end to broccoli, a linker joins the tracrRNA at the 3′ end to target sequence, followed by crRNA; orientation 3, which comprises a target, crRNA, tracrRNA and a long linker at the 3′ end of the tracrRNA only.

FIG. 11A and FIG. 11B depict a series of graphs that show base editing using either modified or unmodified linear or circular guide RNAs.

FIG. 12A illustrates schematically the design of a messenger RNA molecule that is circularized by T4 RNA ligase 2. FIG. 12B and FIG. 12C depict schematically the design of messenger RNA molecules that are circularized by T4 RNA ligase 1. FIG. 12D is a schematic which shows two methods of synthesis of circular RNA using T4 RNA ligase 1 or T4 RNA ligase 2. In the first method, 5′ terminal triphosphate in messenger RNA is cleaved using RppH to generate messenger RNA comprising 5′ terminal monophosphate. In the second method, in vitro transcription of messenger RNA is carried out in the presence of guanosine monophosphate (GMP) to produce messenger RNA comprising 5′ terminal monophosphate. The linear messenger RNA comprising 5′ terminal monophosphate is then ligated by T4 RNA ligase 1 or T4 RNA ligase 2. FIG. 12E is a denaturing polyacrylamide gel showing slower migrating circular RNA molecules obtained by circularization of linear messenger RNAs depicted in FIG. 12A, FIG. 12B and FIG. 12C in the presence of ligase enzyme, with or without RppH, i.e. using one of the methods depicted in FIG. 12D.

FIG. 13A depicts a gel showing exemplary linearized and corresponding non-linearized template RNAs, e.g. 990, 991, 992, 259, 260 and 261. FIG. 13B depicts a gel with in vitro transcribed exemplary RNAs 990, 991 and 992 with guanosine monophosphate priming at exemplary GTP:GMP ratios of 1:2, 1:4 or 1:10. FIG. 13C is a schematic of an enzymatic method of circularizing mRNA which includes in vitro transcription using GMP priming, circularizing using T4 RNA ligase 2, RNAse digestion and gel electrophoresis. FIG. 13D depicts a gel showing RNA product circularized by an enzymatic method. In vitro transcribed RNA with exemplary GTP:GMP ratios of 1:2, 1:4 or 1:10 were treated with RNAse R and/or ligase. The gel showed RNAse resistant circular RNA species in lanes 4, 9 and 13. FIG. 13E depicts a schematic of a circularization method carried out by a self-splicing mechanism which comprises the steps of in vitro transcription by 5′ triphosphate, circularization by self-splicing introns, and subsequent RNase digestion with RNAse R and/or 2 mM GTP, followed by gel electrophoresis. FIG. 13F is a gel that showed RNA product circularized by Anabaena intron, and that WT T4 and mutated T4 did not circularize product. FIG. 13G is a schematic of the mechanism of self-circularization by Anabaena intron. FIG. 13H is a schematic of the circularization of RNA by RT-PCR using divergent primers. FIG. 13I is a gel showing RT-PCR product with divergent primers, confirming that the circularization reaction was successful.

FIG. 14 is a graph of Cas12a gene editing efficiency in CD34+ cells depicted as percentage indel mutations using linear and circular guide RNAs.

### DETAILED DESCRIPTION

The present invention provides methods of producing circular guide RNAs (cgRNAs) that have increased stability against ubiquitous cellular exonucleases without requiring non-natural modifications. Due to increased stability, cgRNAs show improved frequency of gene editing and reduced off-target effects. In some aspects, methods of producing cgRNA comprise use of enzymatic or chemical ligation, and/or ribozymes for circularization. The resultant cgRNA is obtained with high purity, yield and integrity.

Circular gRNAs (cgRNAs) have potential numerous advantages that include, for example increased stability. Circular guides are at least as stable as end modified gRNA but do not require non-natural modifications. cgRNA also are advantageous because of ease of purification in comparison to linear gRNA. For example, purification of cgRNA is simpler at least due to the presence of a 5′ phosphate—a property that could enable simple cleanup and purification away from linear RNA. cgRNA also are advantageous because of the ability to synthesize cgRNA fully enzymatically, thus not requiring chemical synthesis. This in turn allows for a significant advancement in manufacturing.

In some aspects, provided herein are methods of producing other types of circular RNAs, including but not limited to, circular or cyclic messenger RNA (used interchangeably herein), and compositions comprising the same. Circular messenger RNAs have increased stability against exonucleases and thereby have extended half-lives for protein translation. Various aspects of the invention are described in detail in the following sections. The use of sections is not meant to limit the invention. Each section can apply to any aspect of the invention. In this application, the use of “or” means “and/or” unless stated otherwise.

Guide RNA (gRNA)

As used herein, guide RNA (gRNA) also refers to circular guide RNA (cgRNA) unless otherwise noted. A gRNA comprises a polynucleotide sequence complementary to a target sequence. The gRNA hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or 100% complementarity to a target nucleic acid sequence.

In some embodiments, the gRNA is between about 50 nucleotides and 250 nucleotides. In some embodiments, the gRNA is between about 50 nucleotides and 500 nucleotides. In some embodiments, the gRNA is between about 50 nucleotides and 1,000 nucleotides. In some embodiments, the gRNA is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides long. In some embodiments, the gRNA of is between about 50 and 75 nucleotides long. In some embodiments, the gRNA is between about 75 and 100 nucleotides long. In some embodiments, the gRNA is between about 100 and 125 nucleotides long. In some embodiments, the gRNA is between about 125 and 150 nucleotides long. In some embodiments, the gRNA is between about 150 and 175 nucleotides long. In some embodiments, the gRNA is between about 175 and 200 nucleotides long. In some embodiments, the gRNA is between about 200 and 225 nucleotides long. In some embodiments, the gRNA is between about 225 and 250 nucleotides long.

In some embodiments, the gRNA comprises a ligated crRNA and a tracrRNA. Various crRNA and tracrRNA sequences are known in the art, for example those associated with several type II CRISPR-Cas9 systems (e.g., WO2013/176772), Cpf1, SaCas, and Cas12, among others.

A gRNA can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustIX, BLAST, Novoalign, SOAP, Maq, and ELAND.

In some embodiments, a gRNA is designed to target to a unique target sequence within the genome of a cell. In some embodiments, a gRNA is designed to lack a PAM sequence. In some embodiments, a gRNA sequence is designed to have optimal secondary structure using a folding algorithm including mFold or Geneious. In some embodiments, expression of gRNAs may be under an inducible promoter, e.g. hormone inducible, tetracycline or doxycycline inducible, arabinose inducible, or light inducible.

In some embodiments, the gRNA sequence is a “dead crRNAs,” “dead guides,” or “dead guide sequences” that can form a complex with a CRISPR-associated protein and bind specific targets without any substantial nuclease activity.

In some embodiments, the gRNA is chemically modified in the sugar phosphate backbone or base. In some embodiments, the gRNA has one or more of the following modifications 2′O-methyl, 2′-F or locked nucleic acids to improve nuclease resistance or base pairing. In some embodiments, the gRNA may contain modified bases such as 2-thiouridiene or N6-methyladenosine.

In some embodiments, the gRNA is conjugated with other oligonucleotides, peptides, proteins, tags, dyes, or polyethylene glycol.

In some embodiments, the gRNA includes an aptamer or riboswitch sequence that binds specific target molecules due to their three-dimensional structure.

In some embodiments, gRNA has two, three, four or five hairpins.

In some embodiments, gRNA includes a transcription termination sequence, which includes a polyT sequences comprising six nucleotides.

### Production of Circular Guide RNA

The described methods produce cyclic or circular guide RNA (used interchangeably in this application) (cgRNA) that has beneficial properties of high stability, purity, integrity and yield.

The ligation strategies described herein differ from previously-reported chemical ligation strategies used to synthesize synthetic RNAs, such as gRNAs, as the described strategies form natural phosphate linkage at the site of ligation.

Production of gRNA for Circularization or Cyclization

Various methods for the production of guide RNA can be used, including for example, synthetic methods (e.g., solid phase synthesis “SPS”) and/or enzymatically-derived guide RNA (e.g., using T7 RNA polymerase). Either synthetically-derived or enzymatically-derived guide RNA can be used in the methods to produce cgRNA. Two general approaches relating to the production of cgRNA include: 1) using short DNA splints to pre-organize ends of linear gRNA for ligation by a nick-joining ligase (e.g., T4 RNA ligase 2); and 2) using single-stranded RNA ligase (e.g., T4RNA ligase 1) to join untemplated ends of RNA.

In some embodiments, cgRNA is synthesized starting from a linear guide RNA. In some embodiments, linear gRNA is produced by in vitro transcription using T7 RNA polymerase, Syn5 polymerase, VSW3 RNA polymerase, or another RNA polymerase.

In some embodiments, linear gRNA is produced by in-cell transcription by RNA Polymerase III (U6) or a modified RNA polymerase III such as U6+27 RNA polymerase III.

In some embodiments, linear gRNA is produced by in cell transcription by RNA Polymerase I or RNA Polymerase II.

A segmented synthetic approach is advantageous in producing linear gRNA because short sections of RNA can be produced with greater purity post-purification compared to full length gRNA. In this approach the 5′ acceptor RNA is the smallest RNA fragment (about 30-50 nts) and can thus be purified to a high level before ligation. The 3′ donor RNA is terminated with a phosphate that is required for synthesis and thus only the full-length fragment will be incorporated into the full length product (i.e., truncations are not substrates).

The advantages are increased when considering gRNAs that are greater than 100 nts, such as pegRNA or Cas12b guides. The types of enzymatic ligations described herein are very high yielding (>80%) and the oligonucleotide starting material can be separated from ligated product with high selectivity, ensuring that full-length product is very pure. These types of enzymatic ligations are relatively inexpensive and scale well.

Other means of linear RNA synthesis, include fully synthetic, and RNA-dependent RNA transcription.

Enzymatic Ligation to Produce Circular Guide RNA (cgRNA)

Using an enzymatic approach to cyclization, a cgRNA is produced using enzymatic methods. In some embodiments, the method comprises contacting a linear guide RNA comprising a phosphate at the 5′-terminus with a ligating enzyme to bring together a first end and a second end of the guide RNA thus creating a cgRNA.

The ligation approach described herein makes use of ligases, a class of enzymes that combine sections of nucleic acids with each other. An advantage of using such ligases is that the resulting linkages between the fragments of RNA are indistinguishable from naturally occurring RNA or DNA. The ligases function on RNA or DNA and perform reactions with high efficiency.

Various ligases can be used with the methods described herein. For example, one or more of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, Splint® Ligase E. coli DNA Ligase, 9°N DNA Ligase, CircLigase, CircLigase II, DNA Ligase I, DNA Ligase III, and DNA Ligase IV can be used. In some embodiments, T4 RNA ligase 1 is used to ligate the first end and the second end of the guide RNA. In some embodiments, T4 RNA ligase 2 is used for ligating the first end and the second end of the guide RNA.

In some embodiments, two or more RNA fragments are ligated using a self-templating approach, followed by cyclization to create a cgRNA. For example, in some embodiments, 2, 3, 4, 5, 6, 7, 8, 9, 10 or more RNA fragments are ligated, followed by cyclization to create a cgRNA. In some embodiments, producing a synthetic cgRNA comprises providing two or more RNA fragments; providing an oligonucleotide that has partial complementarity to the two or more RNA fragments, wherein the complementarity of the oligonucleotide allows for base pairing with the two or more RNA fragments; and providing a ligase to catalyze ligation between the two or more RNA fragments, thus producing a synthetic circular guide RNA (cgRNA).

Various kinds of ligation are possible using this approach, such as ligation within a terminal loop of a hairpin formed between the first RNA and the second RNA. Various ligases are suitable for ligation at the terminal loop of a hairpin formed, such as T4 RNA ligase 1. Another kind of ligation that is possible with this approach is ligation within the duplex formed between the first RNA and the second RNA. Various ligases are suitable for ligating at the duplex formed between the two RNAs, such as T4 RNA ligase 2 and DNA ligases.

In some embodiments, the cgRNA comprises a crRNA. In some embodiments, the cgRNA comprises a tracrRNA. In some embodiments, the cgRNA comprises a crRNA and a cgRNA.

In some embodiments, a linear guide RNA is first synthesized. In this approach, two or more separate RNAs are ligated together. In some embodiments, a first RNA comprises a trans-activating RNA (tracrRNA), and a second RNA comprises a clustered regularly interspersed short palindromic repeats (CRISPR) RNA (crRNA).

In some embodiments, the RNA comprising the tracrRNA sequences are synthesized such that a portion of the tracrRNA contains a phosphate at the 5′-terminus. Two forms of ligation are possible with this approach, both of which are found within the stem loop region. The first form of ligation occurs within the terminal loop of the hairpin, which is a natural site of T4 RNA Ligase 1. The second form of ligation occurs within the duplex which is a natural of T4 RNA Ligase 2 and DNA ligases. One of the advantages of this form of ligation is that fragment impurities are readily removable because of the marked differences in elution time between the fused gRNA and the fragment impurities.

1. Oligonucleotide Splint or Template Approach for the Production of cgRNAs

In some embodiments, the RNA fragments meant for ligation can be brought into physical proximity for the ligation reaction by use of a nucleic acid template that has complementarity to a first end and a second end of an RNA fragment. This template is referred to herein as an oligonucleotide splint. In some embodiments, an oligonucleotide splint is used in the production of the synthetic circular guide RNAs (cgRNAs). The use of an oligonucleotide splint allows for one end of an RNA molecule to be brought into physical proximity to a second end for the reaction. Oligonucleotide splints can be designed with imperfect pairing to generate loops that are amenable to ligation by a particular ligase, e.g., T4 RNA Ligase 2. In some embodiments, oligonucleotide splints are used to bring together more than two fragments of RNA.

The oligonucleotide splints can be any suitable polymer that is capable of bringing the one or more RNA molecules in close proximity can be used. For example, in some embodiments, the oligonucleotide splint is an RNA molecule or a DNA molecule.

In some embodiments, the oligonucleotide splint has complementarity to sections of the first end of the RNA and the second end of the RNA. The complementarity can either be partial or perfect. Accordingly, in some embodiments, a method of producing a synthetic circular guide RNA is provided comprising providing a linear RNA comprising a 5′ phosphate and a second end comprising free 3′-hydoxyl; providing an oligonucleotide that has partial complementarity to the first end of the linear guide RNA and the second end of the linear guide RNA, wherein the complementarity of the oligonucleotide allows for base pairing with the first end and the second end of the guide RNA; and providing a ligase to catalyze ligation between the first end and the second end of the guide RNA, thus producing a circular guide RNA (cgRNA). In some embodiments, the oligonucleotide splints are about 15-40 nucleotides long.

In some embodiments, the oligonucleotide splint has no complementarity to the sections of the first end of the linear guide RNA and the second end of the linear guide RNA that will be coupled. Accordingly, in some embodiments, a method of producing a circular guide RNA, is provided comprising providing a linear guide RNA comprising a 5′ phosphate; and a free 3′-hydroxyl; providing an oligonucleotide that has no complementarity to nucleotides of the first end of the guide RNA and the second end of the guide RNA that will be coupled; and providing a ligase to catalyze ligation between the first end and the second end of the guide RNA, thus producing a cgRNA.

In some embodiments, one or more oligonucleotide splints anneal to the first end and to the second end of the guide RNA thereby facilitating the formation of a loop structure between the first end and the second end of the guide RNA. In some embodiments, the oligonucleotide splint hybridizes with at least 6-30 nucleotides at the first end of the guide RNA. In some embodiments, the oligonucleotide splint hybridizes with at least 6-30 nucleotides at the second end of the guide RNA. In some embodiments, the oligonucleotide splint has perfect complementarity with at least 6-30 nucleotides at the first end and the second end of the guide RNA. In some embodiments, the ligating enzyme is T4 RNA Ligase 2.

II. Self-Templating Approach for Production of cgRNAs

In some embodiments, a self-templating approach is used to produce circular guide RNAs. In some embodiments of the self-templating approach, a linear guide RNA is provided that has a first end and a second end that shares partial complementarity.

In some embodiments, the RNA fragments can be associated by base-pairing with each other before the ligation reaction. This approach is referred to herein as “self-templating.” In some embodiments, the ligation reaction can proceed with high yield without the need for physical association of the RNA sections beforehand. In view of this, select ligation reactions do not require the use of an oligonucleotide splint and are self-templating. In some embodiments, the partial complementarity between the first end and the second end comprises complementarity of about 10-20 nucleotides. In some embodiments, the ligating enzyme is a single-strand ligase. In some embodiments, the single-strand ligase is T4 RNA Ligase 1. In some embodiments, addition of the ligating enzyme promotes cyclization by hybridization of the partially complementary ends in the absence of any template or oligonucleotide splint.

In some embodiments, using the self-templating approach, the locations of the stem loop that can be selected for ligation of RNA fragments include the loop or in the helix where one of the oligos contains a short stem-loop (e.g., self-templating nick). Nicks can be included on splints, overhangs, blunt ends, and bulges can also be used.

Thus, in some aspects, the self-templating approach of producing a circular guide RNA comprises providing a linear guide RNA comprising a 5′-monophosphate; with partial complementarity to the 3′ end; and providing a ligase to catalyze ligation between the first end and the second end of the RNA, thus producing a cgRNA.

### Self-Circularizing RNAs Using Self-Splicing Ribozymes

In some embodiments, guide RNAs are circularized using a circularizing technique that employs self-splicing ribozymes. In some embodiments, guide RNAs are circularized using intronic ribozymes.

In some aspects, a method of making circular guide RNA (cgRNA) is provided comprising: modifying the ends of a linear guide RNA to create a first 5′ hydroxyl end and a second 2′-3′ cyclic phosphate end; and ligating the first end and the second end with an RNA ligase thereby generating a cgRNA. The modifying of the ends can be performed using ribozymes. Various kinds of ribozymes can be used and including, for example, a self-spicing ribozyme. In some embodiments the self-splicing ribozyme is a twister ribozyme.

In some embodiments, guide RNAs are circularized using Twister ribozymes. In some embodiments, twister ribozymes are used to generate specific ends (5′ hydroxyl and 2′-3′ cyclic phosphate) in an RNA transcript in cis. Following this, the ends are ligated by RtcB, a ubiquitous and endogenous RNA ligase. The self-circularizing guides are delivered as DNA plasmids driven by U6 promoter, where both cleavage and ligation steps take place directly in transfected cells, or transcribed in vitro using a T7 promoter, then transfected following in vitro ligation with purified RtcB.

Cloning Constructs that Contain an RNA Aptamer or RNA of Interest

Briefly, in some embodiments, two Tornado plasmids are employed. In some embodiments, a first Tornado plasmid comprises an RNA sequence of interest that includes a 5′ NotI and 3′ SacII restriction digestion site, between which a fluorogenic aptamer (for example, such as Broccoli) has been inserted. Any circular RNA aptamer or sequence may be cloned in with NotI and SacII, replacing the broccoli sequence.

In some embodiments, a second Tornado plasmid, called Tornado-F30, comprises a Broccoli sequence within a folding scaffold called F30. F30 is a 3-way RNA junction derived from a naturally occurring phage RNA, and aptamers inserted (via an internal stem of the aptamer) into an arm of F30 fold more efficiently. F30 contains 3 stems, allowing for expression of bifunctional circular RNAs. One F30 stem is continuous with the stem required for circular RNA maturation, while the other two F30 stems are not involved in maturation. The loops of these two stems can be replaced with any two desired sequences. In this way the RNA can contain a separate aptamer on either “arm”. The sequence on either arm can be cloned in or out using restriction digest cloning.

In the Tornado-F30 construct, the first F30 arm is extended by two RsrII restriction sites that flank a Broccoli aptamer. The Broccoli aptamer is used to validate that the circular RNA is uniformly (i.e. high transfection efficiency) and abundantly expressed by cells. Cells are transfected, total cellular RNA is extracted and run on a gel and stained with Broccoli's fluorophore (DFHBI-IT). Broccoli is replaced on the first arm by cutting with RsrII and inserting a sequence with RsrII sites on either side. The second F30 arm is capped by a KflI restriction site. A new sequence can be introduced at this arm by using a cloning insert that is flanked by KflI a sequence on both ends. Sequences of Tornado and Tornado F30 plasmids are provided in Table 1. In some embodiments, Tornado F30 is not used.

TABLE 1
Tornado and Tornado F30 Plasmid Sequences
Tornado
GAGGGCCTATTTCCCATGATTCCTTCATATTTGCATATACGATACAAGGCTGTTAGAGAGA
TAATTAGAATTAATTTGACTGTAAACACAAAGATATTAGTACAAAATACGTGACGTAGAAA
GTAATAATTTCTTGGGTAGTTTGCAGTTTTAAAATTATGTTTTAAAATGGACTATCATATG
CTTACCGTAACTTGAAAGTATTTCGATTTCTTGGCTTTATATATCTTGTGGAAAGGACGAA
ACACC<GTGCTCGCTTCGGCAGCACATATACTAGTCGACGGGCCGCACTCGCCGGTCCCAA
GCCCGGATAAAATGGGAGGGGGCGGGAAACCGCCT\hat{ }AACCATGCCGAGTGCGGCCGCGAGA
CGGTCGGGTCCAGATATTCGTATCTGTCGAGTAGAGTGTGGGCTCGTGGCCGCGGTCGGCG
TGGACTGTAG\hat{ }AACACTGCCAATGCCGGTCCCAAGCCCGGATAAAAGTGGAGGGTACAGTC
CACGCTCTAGAGCGGACTTCGGTCCGCTTTT> (SEQ ID NO: 1)
Tornado-F30
GAGGGCCTATTTCCCATGATTCCTTCATATTTGCATATACGATACAAGGCTGTTAGAGAGA
TAATTAGAATTAATTTGACTGTAAACACAAAGATATTAGTACAAAATACGTGACGTAGAAA
GTAATAATTTCTTGGGTAGTTTGCAGTTTTAAAATTATGTTTTAAAATGGACTATCATATG
CTTACCGTAACTTGAAAGTATTTCGATTTCTTGGCTTTATATATCTTGTGGAAAGGACGAA
ACACC<GTGCTCGCTTCGGCAGCACATATACTAGTCGACGGGCCGCACTCGCCGGTCCCAA
GCCCGGATAAAATGGGAGGGGGCGGGAAACCGCCT\hat{ }AACCATGCCGAGTGCGGCCGCTTGC
CATGTGTATCGGTCCGGAGACGGTCGGGTCCAGATATTCGTATCTGTCGAGTAGAGTGTGG
GCTCCGGTCCGATACTCTGATGATGGGTCCCATCATTCATGGCAAGTGGCCGCGGTCGGCG
TGGACTGTAG\hat{ }AACACTGCCAATGCCGGTCCCAAGCCCGGATAAAAGTGGAGGGTACAGT
CCACGCTCTAGAGCGCACTTCGGTGCGCTTTT> (SEQ ID NO: 2)
U6+27 promoter is underlined.
5′ and 3′ ribozyme sequences are in italics.
F30 sequences are in italics and underlined.
RNA sequence of interest (Broccoli aptamer) is in bold and italics.
The U6 terminator is in bold.
The transcription start (<) and stop (>) sites.
The \hat{ } symbol represents a cleavage site.
Sites used for cloning are bold and underlined (SaII: GTCGAC, XbaI: TCTAGA, NotI: GCGGCCGC, SacII: CCGCGG, RsrII: CGGTCCG and KflI: GGGTCCC)

The KflI cut site is GG/GWCCC (W=A or T) while the RsrII enzyme cut site is CG/GWCCG. Since each site is an odd number of bases long, they are directional cloning sites, reducing reverse direction insertions during cloning. Both enzymes allow directional cloning into this single site using one enzyme. When an insert is inserted on either arm, two RsrII or KflI sequences flank the RNA such that they base pair and form a continuous stem because both RsrII and KflI restriction sites are palindromic.

Ligation of the RNA ends occurs immediately after cleavage. The sequence of the circular RNA is determined from the beginning and end of the transcript just after cleavage.

Before cloning an aptamer or other RNA into the Tornado expression cassette, the sequence is scanned for consecutive uridines. Since the U6 promoter is used, polymerase III is recruited for transcription and the termination signal for polymerase III is traditionally poly (U). Since four or more uridines can cause high levels of termination (4 Us is a weak terminator, and 5+ Us is a strong terminator), poly (U) stretches are mutated to ensure that full length RNA is transcribed and that the RNA is able to circularize properly. Sequence confirmation of DNA after cloning is typically performed following screening of individual bacterial colonies.

HEK293T cells are transfected with plasmid, cellular RNA is harvested and total RNA is isolated. Circular RNA is detected as early as 16 hours after transfection, but only reaches maximum levels after 3 days in HEK293T. Confirmation that RNA is circular is obtained from its ability to resist degradation. Actinomycin D is used to measure resistance to degradation. Actinomycin D intercalates in DNA preventing transcription. Without continuous transcription of polymerases, the levels of all RNAs drop due to degradation. Circular RNAs demonstrate high stability in this assay. Untreated cells are compared with those treated with 5 μg/ml actinomycin D for 6 hours prior to RNA harvesting.

Total cellular RNA is separated by denaturing urea PAGE; however, the exact mobility of circular RNA by PAGE is difficult to predict. While their mobility is correlated with their size, circular RNAs do not migrate as would be expected based on nucleotide length. In a 6% acrylamide gel, small circular RNAs (roughly less than 200 nt) run faster than expected, while longer RNAs (roughly over 200 nt) run slower than expected. This roughly 200 nt delineation between the behavior of small and large circular RNAs also changes depending on the acrylamide percentage of the gel.

An alternate approach is Broccoli imaging of PAGE gels by staining with DFHBI-IT and confirming that they do not degrade upon Actinomycin D treatment.

By using this approach, circular guide RNAs are generated using self-splicing ribozymes without requiring a template.

In some embodiments, provided herein is a method of making circular RNA, wherein the method comprises the steps of in vitro transcription in the presence of guanosine monophosphate, generating 5′ triphosphate, followed by circularization by self-splicing introns. In some embodiments, the self-splicing intron is Anabaena intron. In some embodiments, the Anabaena intron comprises SEQ ID NOs: 24 and 25. In some embodiments, the self-splicing intron is T4 intron. In some embodiments, the T4 intron comprises SEQ ID NOs: 26 and 27. In some embodiments, the T4 intron is modified. In some embodiments, the intron comprises SEQ ID NOs: 28 and 29.

In some embodiments, circularization is carried out by a self-splicing mechanism which comprises the steps of in vitro transcription in the presence of guanosine monophosphate generating RNA with 5′ triphosphate, circularization by self-splicing introns, and subsequent RNase digestion with RNAse R and/or 2 mM GTP, followed by gel electrophoresis. In some embodiments, a self-splicing intron using permuted group I catalytic intron is used to circularize RNA. In some embodiments, GTP and Mg2+ are used as cofactors. Three different self-splicing introns (Anabaena, WT T4 and mutated T4) were designed for template circularization. Briefly, circular RNA is synthesized in vitro in which transposed halves of a split group I intron flank the sequence of the RNA to be circularized. Exemplary permuted intron-exon (PIE) sequences for self-splicing introns are as shown in Table 9. (Wesselhoeft, R A et al., Nature Communications. 2018. 9:2629, p. 1-10; Rausch et al., Nucleic Acids Research. 2021. 49 (6). p. 1-13). The permuted intron-exon splicing strategy comprises fused partial exons flanked by half-intron sequences which undergo double transesterification reactions characteristic of group I catalytic introns, but because the exons are fused they are excised as covalently linked circular RNA from 5′ to 3′, thus yielding circular RNA.

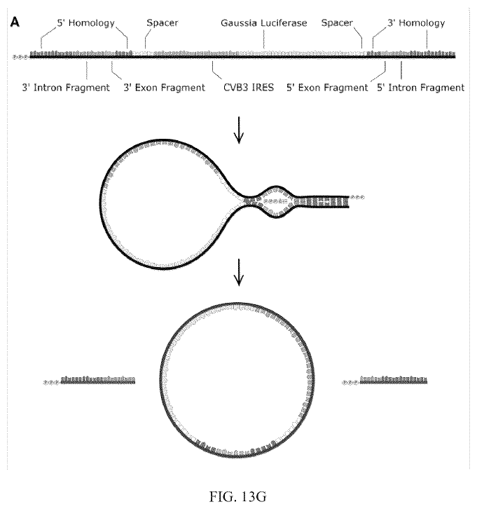

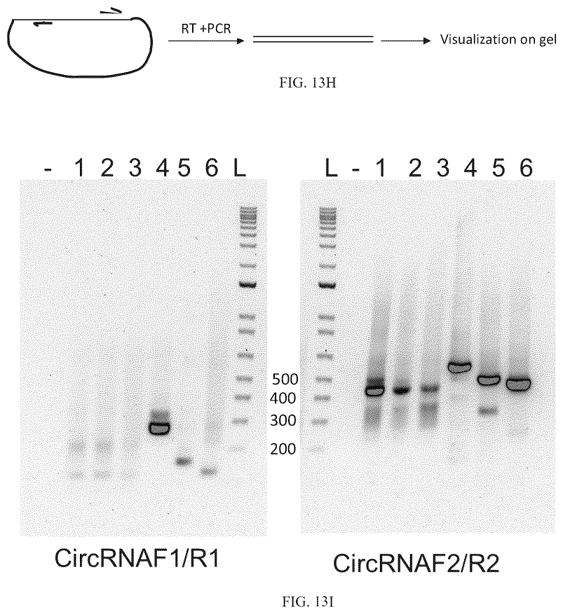

A schematic of the mechanism of self-circularization by Anabaena intron is shown in FIG. 13G.

Without wishing to be bound by any particular theory, it is contemplated that Anabaena catalytic intron from Anabaena pre-tRNA results in a weakening of a short stretch of homology between the Internal Ribosome Entry Site (IRES) and the 3′ end of the coding region, which aids in the formation of an isolated splicing bubble. The Anabaena intron system used herein (SEQ ID NO: 24 and 25) strengthens this region of internal homology increasing splicing efficiency. The Anabaena intron also resulted in reduction in circRNA nicking compared to T4 intron. Without wishing to be bound by any particular theory, it is contemplated the Anabaena system successfully generates circular RNA due to increased splicing efficiency and generation of intact circular RNA without nicks.

### Chemically Modified Circular Guide RNA

In some embodiments, the first end of the guide RNA and/or the second end of the guide RNA comprises a chemical modification to its backbone or to one or more of its bases. For example, chemically modified RNA can comprise chemical synthesis can be used to install highly modified monomers including modified sugars, bases, backbones or functional groups that do not resemble natural nucleotides.

Accordingly, in some embodiments, the first end of the guide RNA and/or the second end of the guide RNA comprises a modified base. In some embodiments, the modified RNA include one or more of the following 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′-O-Methyl modifications can also be used with the methods described herein. For example, the following RNA comprising one or more of the following 2′OMethyl modifications can be used with the methods described: 2′-OMe-5-Methyl-IC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the first end of the guide RNA and/or second end of the guide RNA comprises one or more of the following modifications: phosphorothioates, 2′O-methyl, 2′ fluoro (2′F), DNA.

In some embodiments, the first end of the guide RNA and/or the second end of the guide RNA comprises 2′OMe modifications at the 3′ and 5′-ends.

In some embodiments, the first end of the guide RNA and/or second end of the guide RNA comprises one or more of the following modifications: 2′-O-2-Methoxyethyl (MOE), locked nucleic acids, bridged nucleic acids, unlocked nucleic acids, peptide nucleic acids, morpholino nucleic acids.

In some embodiments, the first end of the guide RNA and/or second end of the guide RNA comprises one or more of the following base modifications: 2,6-diaminopurine, 2-aminopurine, pseudouracil, N1-methyl-psuedouracil, 5′ methyl cytosine, 2′pyrimidinone (zebularine), thymine.

Other modified bases include for example, 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Desoxy-r(A,C,G,U), 3′-O-Methyl-r(A,C,G,U), IT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the first end of the guide RNA and/or second end of the guide RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

### RNA Ligation

In some embodiments the Tm (melting temperature) of the circular gRNA is greater than 0° C., 1° C., 2° C., 3° C., 4° C., 5° C., 6° C., 7° C., 8° C., 9° C., 10° C., 11° C., 12° C., 12° C., 14° C., 15° C., 16° C., 17° C., 18° C., 19° C., 20° C., 25° C., 30° C., 35° C., 40° C., 45° C., 50° C., 55° C., 60° C., 65° C., 70° C., 75° C., 80° C., 85° C., 90° C., 95° C., 100° C., or more.

In some embodiments, the temperature at which the ligation reaction occurs influences the yield or productivity of the cgRNA ligation reaction. In some embodiments, the temperature at which the ligation reaction occurs is about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C. Accordingly, in some embodiments, the temperature at which the ligation reaction occurs is about 15° C. In some embodiments, the temperature at which the ligation reaction occurs is about 16° C. In some embodiments, the temperature at which the ligation reaction occurs is about 17° C. In some embodiments, the temperature at which the ligation reaction occurs is about 18° C. In some embodiments, the temperature at which the ligation reaction occurs is about 19° C. In some embodiments, the temperature at which the ligation reaction occurs is about 20° C. In some embodiments, the temperature at which the ligation reaction occurs is about 21° C. In some embodiments, the temperature at which the ligation reaction occurs is about 22° C. In some embodiments, the temperature at which the ligation reaction occurs is about 23° C. In some embodiments, the temperature at which the ligation reaction occurs is about 24° C. In some embodiments, the temperature at which the ligation reaction occurs is about 25° C. In some embodiments, the temperature at which the ligation reaction occurs is about 26° C. In some embodiments, the temperature at which the ligation reaction occurs is about 27° C. In some embodiments, the temperature at which the ligation reaction occurs is about 28° C. In some embodiments, the temperature at which the ligation reaction occurs is about 29° C. In some embodiments, the temperature at which the ligation reaction occurs is about 30° C. In some embodiments, the temperature at which the ligation reaction occurs is about 31° C. In some embodiments, the temperature at which the ligation reaction occurs is about 32° C. In some embodiments, the temperature at which the ligation reaction occurs is about 33° C. In some embodiments, the temperature at which the ligation reaction occurs is about 34° C. In some embodiments, the temperature at which the ligation reaction occurs is about 35° C. In some embodiments, the temperature at which the ligation reaction occurs is about 36° C. In some embodiments, the temperature at which the ligation reaction occurs is about 37° C. In some embodiments, the temperature at which the ligation reaction occurs is about 38° C. In some embodiments, the temperature at which the ligation reaction occurs is about 39° C. In some embodiments, the temperature at which the ligation reaction occurs is about 40° C.

### Messenger RNA

Synthesis of mRNA

Messenger RNAs (mRNAs) may be synthesized according to any of a variety of known methods. For example, mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application. In some embodiments, a suitable DNA template typically has a promoter, for example a T3, T7 or SP6 promoter, for in vitro transcription, followed by desired nucleotide sequence for desired mRNA and a termination signal.

In some embodiments, mRNA is produced using T3 RNA Polymerase. T3 RNA Polymerase is a DNA-dependent RNA polymerase from the T3 bacteriophage that catalyzes the formation of RNA from DNA in the 5′→3′ direction on either single-stranded DNA or double-stranded DNA, and is able to incorporate modified nucleotide. T3 polymerase is extremely promoter-specific and transcribes only DNA downstream of a T3 promoter. T3 binds to a consensus promoter sequence of 5′-AATTAACCCTCACTAAAGGGAGA-3′ (SEQ ID NO: 24).

In some embodiments, mRNA is produced using T7 RNA Polymerase. T7 RNA Polymerase is a DNA-dependent RNA polymerase from the T7 bacteriophage that catalyzes the formation of RNA from DNA in the 5′→3′ direction. T7 polymerase is extremely promoter-specific and transcribes only DNA downstream of a T7 promoter. T7 binds to a consensus promoter sequence of 5′-TAATACGACTCACTATAGGGAGA-3′ (SEQ ID NO: 25). The T7 polymerase also requires a double stranded DNA template and Mg²⁺ ion as cofactor for the synthesis of RNA. It has a very low error rate.

In some embodiments, mRNA is produced using SP6 RNA Polymerase. SP6 RNA Polymerase is a DNA-dependent RNA polymerase with high sequence specificity for SP6 promoter sequences. The SP6 polymerase catalyzes the 5′→3′ in vitro synthesis of RNA on either single-stranded DNA or double-stranded DNA downstream from its promoter; it incorporates native ribonucleotides and/or modified ribonucleotides and/or labeled ribonucleotides into the polymerized transcript. SP6 binds to a consensus promoter sequence of 5′-ATTTACGACACACTATAGAAGAA-3′ (SEQ ID NO: 26). Examples of such labeled ribonucleotides include biotin-, fluorescein-, digoxigenin-, aminoallyl-, and isotope-labeled nucleotides.

### DNA Template

Typically, a DNA template is either entirely double-stranded or mostly single-stranded with a suitable promoter sequence (e.g. T3, T7 or SP6 promoter).

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription, provided that they contain a double-stranded promoter upstream (and in the correct orientation) of the DNA sequence to be transcribed.

In some embodiments, the linearized DNA template has a blunt-end.

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature poly A sites, Shine-Dalgarno (SD) sequences, and/or other elements relevant to translation; and/or the DNA sequence may be optimized regarding codon context, codon-anticodon interaction, translational pause sites, and/or other elements relevant to protein folding.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

In some embodiments, 1-100 mg of RNA polymerase is typically used per gram (g) of mRNA produced. In some embodiments, the concentration of the RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the RNA polymerase is from about 10 to 50 nM, 20 to 50 nM, or 30 to 50 nM. A concentration of 100 to 10000 Units/ml of the RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

Linear or linearized DNA template (e.g., as described above and in an amount/concentration sufficient to provide a desired amount of RNA), the RNA polymerase reaction buffer, and RNA polymerase are combined to form the reaction mixture. The reaction mixture is incubated at between about 37° C. and about 42° C. for thirty minutes to six hours, e.g., about sixty to about ninety minutes.

In some embodiments, about 5 mM NTPs, about 0.05 mg/mL RNA polymerase, and about 0.1 mg/ml DNA template in a suitable RNA polymerase reaction buffer (final reaction mixture pH of about 7.5) is incubated at about 37° C. to about 42° C. for sixty to ninety minutes.

In some embodiments, a reaction mixture contains linearized double stranded DNA template with an RNA polymerase-specific promoter, RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10× is 100 mM Tris-HCl, 5 mM MgCl₂ and 25 mM CaCl₂), pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and RNA polymerase at a concentration ranging from 0.01-0.1 mg/ml, e.g., the reaction mixture comprises NTPs at a concentration of 5 mM, the DNA template at a concentration of 0.1 mg/ml, and the RNA polymerase at a concentration of 0.05 mg/ml.

### Nucleotides

Various naturally-occurring or modified nucleosides may be used to produce mRNA. In some embodiments, an mRNA comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), N6-methyladenosine, pseudouridine (“VU”), and/or 2-thio-uridine (“2sU”). The mRNA may be RNA, which is defined as RNA in which 25% of U residues are 2-thio-uridine and 25% of C residues are 5-methylcytidine. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

### Post-Synthesis Processing

Typically, a 5′ cap and/or a 3′ tail may be added to linear mRNA after the synthesis. Circular mRNA is generated from linear mRNA that lacks a 5′ cap, or a 3′ tail, i.e. circular mRNA is generated from linear mRNA without post-synthesis processing.

In linear mRNA, the presence of the cap is important in providing resistance to nucleases found in most eukaryotic cells. The presence of a “tail” serves to protect mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G (5′) ppp (5′ (A,G (5′) ppp (5′) A and G (5′) ppp (5′) G.

The presence of a “tail” at 3′ end serves to protect the mRNA from exonuclease degradation. The 3′ tail may be added before, after or at the same time of adding the 5′ Cap. In some embodiments, the poly A tail is added co-transcriptionally. In some embodiments, the poly A tail is added post-transcriptionally. In some embodiments, the poly C tail is added co-transcriptionally. In some embodiments, the poly C tail is added post-transcriptionally. Typically, a tail structure includes a poly A and/or poly C tail.

### Production of Circular or Cyclic Messenger RNA

In some aspects, described herein are methods to produce circular or cyclic messenger RNA, used interchangeably herein, and compositions comprising the same. Circular RNAs typically have improved stability relative to linear RNAs since they lack free ends necessary for exonuclease-mediated degradation and RNA turnover. The improved stability and extended half-life improves overall efficacy of exogenous circular mRNA in a variety of applications. Circularizing long in vitro transcribed messenger RNAs, purifying circular mRNA and achieving high protein expression is challenging. RNA circularization is typically carried out by chemical methods using cyanogen bromide or a similar condensing agent, enzymatic methods using RNA or DNA ligases, or using ribozymes in methods using self-splicing introns.

In some aspects, provided herein are methods to circularize messenger RNA. In some embodiments, linear messenger RNA generated by in vitro transcription is used to synthesize circular RNA by enzymatic ligation to join the ends of messenger RNA. In some embodiments, ligation methods use T4 RNA ligase 1 or T4 RNA ligase 2. In some embodiments, RNA generated by in vitro transcription that comprises a 5′ terminal triphosphate is treated with RNA pyrophosphohydrolase (RppH) enzyme which cleaves diphosphate residues yielding mRNA with a terminal 5′ monophosphate. In some other embodiments, in vitro transcription is carried out in the presence of guanosine monophosphate (GMP) resulting in mRNA product that is a mixture of 5′ triphosphorylated mRNA and 5′ monophosphorylated mRNA. In some other embodiments, heat annealing is carried out. Messenger RNA comprising 5′ monophosphate is then treated with T4 RNA ligase 1 or T4 RNA ligase 2 to yield circular messenger RNA.

In some embodiments, a ligation method uses a single stranded RNA ligase, T4 RNA ligase 1 to join the untemplated ends of RNA.

In some embodiments, a ligation method uses uses short DNA splints to pre-organize the ends of linear gRNA for ligation by a nick-joining ligase (e.g. T4 RNA ligase 2).

In some aspects, provided herein is a method of making circular RNA comprising: contacting a linear RNA with a ligating enzyme, wherein the contacting brings together a first end and a second end of the RNA, and wherein the ligating enzyme ligates the first and the second end of the RNA thus creating a circular RNA. In some embodiments, the RNA is messenger RNA. In some embodiments, the linear RNA comprises a terminal 5′ phosphate.

In some embodiments, the ligating enzyme is selected from the group consisting of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, Splint® Ligase E. coli DNA Ligase, 9°N DNA Ligase, CircLigase, CircLigase II, DNA Ligase I, DNA Ligase III, and DNA Ligase IV.

In some embodiments, ligating occurs in the absence of a template between the first end and the second end of the RNA. In some embodiments, wherein the first end and the second end of the RNA has partial complementarity. In some embodiments, the partial complementarity between the first end and the second end comprises complementarity of at least 5, 10, 15, or 20 nucleotides.

In some embodiments, the ligating enzyme is a single-strand ligase. In some embodiments, the single-strand ligase is T4 RNA Ligase 1.

In some embodiments, the ligating occurs in the presence of a template between the first end and the second end of the RNA. In some embodiments, the ligating enzyme is T4 RNA Ligase 2.

In some embodiments, the RNA is synthesized by in vitro transcription. In some embodiments, the in vitro transcription is carried out in the presence of guanosine monophosphate (GMP) to produce RNA. In some other embodiments, the in vitro transcribed RNA is treated with pyrophosphohydrolase (RppH) enzyme. In some embodiments, RNA is treated with a ligase. In some embodiments, the ligase is T4 RNA ligase 1 or T4 RNA ligase 2. In some embodiments, the ligase is T4 RNA ligase 1. In some embodiments, the ligase is T4 RNA ligase 2.

In some embodiments, the RNA is between about 100 to 1000 nucleotides long. In some embodiments, the RNA is between about 1000 to 10,000 nucleotides long. In some embodiments, the RNA is between about 1 kb to 10 kb in length. In some embodiments, the RNA is greater than 10 kb long.

In some embodiments, the circular RNA has a length of between about 200 to 1000 nucleotides. In some embodiments, the circular RNA has a length of about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides or greater than about 1000 nucleotides. Accordingly, in some embodiments, the circular RNA has a length of about 100 nucleotides. In some embodiments, the circular RNA has a length of about 200 nucleotides. In some embodiments, the circular RNA has a length of about 300 nucleotides. In some embodiments, the circular RNA has a length of about 400 nucleotides.

In some embodiments, the circular RNA has a length of about 500 nucleotides. In some embodiments, the circular RNA has a length of about 600 nucleotides. In some embodiments, the circular RNA has a length of greater than about 700 nucleotides. In some embodiments, the circular RNA has a length of greater than about 800 nucleotides. In some embodiments, the circular RNA has a length of greater than about 900 nucleotides. In some embodiments, the circular RNA has a length of greater than about 1000 nucleotides. In some embodiments, the circular RNA is circular messenger RNA.

In some embodiments, the circular RNA has a length of between about 1000 to 10,000 nucleotides. In some embodiments, the circular RNA has a length of about 1000 nucleotides, about 1500 nucleotides, about 2000 nucleotides, about 2500 nucleotides, about 3000 nucleotides, about 3500 nucleotides, about 4000 nucleotides, about 4500 nucleotides, about 5000 nucleotides, about 5500 nucleotides, about 6000 nucleotides, about 6500 nucleotides, about 7000 nucleotides, about 7500 nucleotides, about 8000 nucleotides, about 8500 nucleotides, about 9000 nucleotides, about 9500 nucleotides, about 10,000 nucleotides or greater than about 10,000 nucleotides. Accordingly, in some embodiments, the circular RNA has a length of about 1000 nucleotides. In some embodiments, the circular RNA has a length of about 1500 nucleotides. In some embodiments, the circular RNA has a length of about 2000 nucleotides. In some embodiments, the circular RNA has a length of about 2500 nucleotides. In some embodiments, the circular RNA has a length of about 3000 nucleotides. In some embodiments, the circular RNA has a length of about 3500 nucleotides. In some embodiments, the circular RNA has a length of about 4000 nucleotides. In some embodiments, the circular RNA has a length of about 4500 nucleotides. In some embodiments, the circular RNA has a length of about 5000 nucleotides. In some embodiments, the circular RNA has a length of about 5500 nucleotides. In some embodiments, the circular RNA has a length of about 6000 nucleotides. In some embodiments, the circular RNA has a length of about 6500 nucleotides. In some embodiments, the circular RNA has a length of about 7000 nucleotides. In some embodiments, the circular RNA has a length of about 7500 nucleotides. In some embodiments, the circular RNA has a length of about 8000 nucleotides. In some embodiments, the circular RNA has a length of about 8500 nucleotides. In some embodiments, the circular RNA has a length of about 9000 nucleotides. In some embodiments, the circular RNA has a length of about 9500 nucleotides. In some embodiments, the circular RNA has a length of about 10,000 nucleotides. In some embodiments, the circular RNA has a length of greater than about 10,000 nucleotides. In some embodiments, the circular RNA is circular messenger RNA.

In some embodiments, the circular RNA has a length of between about 1 kb to about 10 kb. In some embodiments, the circular RNA has a length of between about 3 kb to about 6 kb. In some embodiments, the circular RNA has a length of greater than about 10 kb. In some embodiments, the circular RNA has a length of about 1 kb. In some embodiments, the circular RNA has a length of about 2 kb. In some embodiments, the circular RNA has a length of about 3 kb. In some embodiments, the circular RNA has a length of about 4 kb. In some embodiments, the circular RNA has a length of about 5 kb. In some embodiments, the circular RNA has a length of about 6 kb. In some embodiments, the circular RNA has a length of about 7 kb. In some embodiments, the circular RNA has a length of about 8 kb. In some embodiments, the circular RNA has a length of about 9 kb. In some embodiments, the circular RNA has a length of about 10 kb.

In some embodiments, provided herein is a composition comprising circular messenger RNA generated by the methods disclosed herein. In some embodiments, provided herein is a kit comprising a circular messenger RNA composition.

Gene Editing Using gRNA

The synthetic cgRNA described herein can be used with a suitable gene editing system for targeted gene editing which can result in a gene silencing event, or an alteration of the expression (e.g., an increase or a decrease) in the expression of a desired target gene. Accordingly, in some embodiments, the synthetic cgRNA described herein can be used in a method for targeted transcription activation, targeted transcription repression, targeted epigenome modification, or targeted genome modification, the method comprising introducing into a eukaryotic cell: (a) a synthetic circular guide RNA (cgRNA) as defined herein; (b) at least one CRISPR/Cas protein or a nucleic acid encoding the at least one CRISPR/Cas protein; wherein interactions between (a) and (b) and a target sequence in chromosomal DNA leads to targeted transcription activation, targeted transcription repression, targeted epigenome modification, or targeted genome modification.

In some embodiments, the synthetic cgRNA described herein can be used in a gene editing system comprising: the synthetic circular guide RNA described herein, wherein the RNA guide comprises a direct repeat sequence and a spacer sequence capable of hybridizing to a target nucleic acid; gene editing protein, and wherein the gene editing enzyme is capable of binding to the RNA guide and of causing a break in the target nucleic acid sequence complementary to the RNA guide.

In some embodiments, the synthetic cgRNA described herein can be used in a gene editing system comprising: the synthetic circular guide RNA described herein, wherein the RNA guide comprises a direct repeat sequence and a spacer sequence capable of hybridizing to a target nucleic acid; and a gene editing protein; wherein the gene editing protein is fused to a deaminase, and wherein the gene editing protein fusion is capable of binding to the RNA guide and of editing the target nucleic acid sequence complementary to the RNA guide.

In some embodiments, the invention provides a method of altering expression of a target nucleic acid in a eukaryotic cell comprising: contacting the cell with a gene editing protein, and the synthetic circular guide RNA described herein, wherein the cgRNA comprises a direct repeat sequence and a spacer sequence capable of hybridizing to the target nucleic acid, and wherein the gene editing protein is capable of binding to the cgRNA and of causing a break in the target nucleic acid sequence complementary to the cgRNA.

In some embodiments, the invention provides a method of altering expression of a target nucleic acid in a eukaryotic cell comprising: contacting the cell with a gene editing protein, and the synthetic cgRNA described herein, wherein the cgRNA comprises a direct repeat sequence and a spacer sequence capable of hybridizing to the target nucleic acid, and wherein the gene editing protein is capable of binding to the cgRNA and editing the target nucleic acid sequence complementary to the cgRNA.

In some embodiments, the invention provides a method of modifying a target nucleic acid in a eukaryotic cell comprising: contacting the cell with a gene editing protein, and the synthetic circular guide RNA described herein, wherein the cgRNA comprises a direct repeat sequence and a spacer sequence capable of hybridizing to the target nucleic acid, and wherein the gene editing protein is capable of binding to the cgRNA and editing the target nucleic acid sequence complementary to the cgRNA.

In some embodiments, the gene editing method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the gene editing method or system comprises a fusion protein with enzymes that can edit DNA sequences by chemically modifying nucleotide bases, including deaminase enzymes that can modify adenosine or cytosine bases and function as site-specific base editors. For example, APOBEC1 cytidine deaminase, which usually uses RNA as a substrate, can be targeted to single-stranded and double-stranded DNA when it is fused to Cas9, converting cytidine to uridine directly, and ADAR enzymes deaminate adenosine to inosine. Thus, ‘base editing’ using deaminases enables programmable conversion of one target DNA base into another. Various base editors are known in the art and can be used in the method and systems described herein. Exemplary base editors are described in, for example, Rees and Liu Nature Review Genetics, 2018, 19 (12): 770-788, the contents of which are incorporated herein.

In some embodiments, base editing results in the introduction of stop codons to silence genes. In some embodiments, base editing results in altered protein function by altering amino acid sequences.

In some embodiments, the synthetic circular guide RNA described herein can be used in a gene editing method or system to modulate transcription of target DNA. In some embodiments, the synthetic circular guide RNA can be used in a gene editing method or system to modulate the expression of a target non-coding RNA, including tRNA, IRNA, snoRNA, siRNA, miRNA, and long ncRNA.

In some embodiments, the synthetic circular guide RNA described herein is used for targeted engineering of chromatin loop structures using a suitable gene editing system. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, the synthetic circular guide RNA described herein is used in conjunction with a gene editing system for correction of pathogenic mutations by insertion of beneficial clinical variants or suppressor mutations.

### Therapeutic Applications

The synthetic circular guide RNA and/or circular mRNA described herein can be used in a gene editing system for various therapeutic applications. Accordingly, in some embodiments, a method of treating a disorder or a disease in a subject in need thereof is provided, the method comprising administering to the subject a synthetic circular guide RNA described herein with a gene editing system. Various gene editing systems are known in the art and include for example CRISPR-Cas9, Cpf1, SaCas, and Cas12, among others. The synthetic circular gRNA or circular mRNA described herein can be used with any gene editing system.

In some embodiments, the synthetic circular guide RNA or circular mRNA described herein can be used in conjunction with a gene editing system to treat various diseases and disorders, e.g., genetic disorders (e.g., monogenetic diseases), diseases that can be treated by nuclease activity, and various cancers, etc.

In some embodiments, the synthetic circular guide RNA or circular mRNA described herein can be used in conjunction with a gene editing system to edit a target nucleic acid to modify the target nucleic acid (e.g., by inserting, deleting, or mutating one or more nucleic acid residues). For example, in some embodiments a CRISPR systems is used with the synthetic gRNA described herein and comprises an exogenous donor template nucleic acid (e.g., a DNA molecule or a RNA molecule), which comprises a desirable nucleic acid sequence. Upon resolution of a cleavage event induced with the CRISPR system, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the synthetic circular guide RNA or circular mRNA described herein is used in conjunction with a gene editing system to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA).

In one aspect, the synthetic circular guide RNA or circular mRNA described herein can be used in conjunction with a gene editing system for treating a disease caused by overexpression of RNAs, toxic RNAs, and/or mutated RNAs (e.g., splicing defects or truncations).

In some embodiments, the synthetic circular guide RNA or circular mRNA described herein can be used in conjunction with a gene editing system to target trans-acting mutations affecting RNA-dependent functions that cause various diseases.

In some embodiments, the synthetic circular guide RNA or circular mRNA described herein can be used in conjunction with a gene editing system to target mutations disrupting the cis-acting splicing codes that can cause splicing defects and diseases.

The synthetic circular guide RNA or circular mRNA described herein can be used in conjunction with a gene editing system can for antiviral activity, in particular against RNA viruses. For example, to target viral RNAs using suitable synthetic RNA guides selected to target viral RNA sequences.

The synthetic circular guide RNA or circular mRNA described herein can be used in conjunction with a gene editing system to treat a cancer in a subject (e.g., a human subject). For example, by targeting a RNA molecule that is aberrant (e.g., comprises a point mutation or are alternatively-spliced) and found in cancer cells to induce cell death in the cancer cells (e.g., via apoptosis).

The synthetic circular guide RNA or circular mRNA described herein can be used in conjunction with a gene editing system to treat an infectious disease in a subject. For example, through targeting a RNA molecule expressed by an infectious agent (e.g., a bacteria, a virus, a parasite or a protozoan) in order to target and induce cell death in the infectious agent cell. The synthetic guide RNA or circular mRNA described herein can be used in conjunction with a gene editing system to treat diseases where an intracellular infectious agent infects the cells of a host subject. In some embodiments, a circular mRNA is used in conjunction with a gene editing system to treat diseases.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, the DNA-targeting RNA and/or site-directed modifying polypeptide and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, a DNA-targeting RNA and/or site-directed modifying polypeptide and/or donor polynucleotide are administered directly to the individual. A DNA-targeting RNA and/or site-directed modifying polypeptide and/or donor polynucleotide may be administered by any of a number of well-known methods in the art for the administration of peptides, small molecules and nucleic acids to a subject. A DNA-targeting RNA and/or site-directed modifying polypeptide and/or donor polynucleotide can be incorporated into a variety of formulations. More particularly, a DNA-targeting RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a DNA-targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a DNA-targeting RNA and/or site-directed modifying polypeptide and/or donor polynucleotide are provided. As discussed above with regard to ex vivo methods, an effective amount or effective dose of a DNA-targeting RNA and/or site-directed modifying polypeptide and/or donor polynucleotide in vivo is the amount to induce a 2 fold increase or more in the amount of recombination observed between two homologous sequences relative to a negative control, e.g. a cell contacted with an empty vector or irrelevant polypeptide. The amount of recombination may be measured by any convenient method, e.g. as described above and known in the art. The calculation of the effective amount or effective dose of a DNA-targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a DNA-targeting RNA and/or site-directed modifying polypeptide and/or donor polynucleotide may be obtained from a suitable commercial source. As a general proposition, the total pharmaceutically effective amount of the a DNA-targeting RNA and/or site-directed modifying polypeptide and/or donor polynucleotide administered parenterally per dose will be in a range that can be measured by a dose response curve.

Therapies based on a DNA-targeting RNA and/or site-directed modifying polypeptide and/or donor polynucleotides, i.e. preparations of a DNA-targeting RNA and/or site-directed modifying polypeptide and/or donor polynucleotide to be used for therapeutic administration, must be sterile. Sterility is readily accomplished by filtration through sterile filtration membranes (e.g., 0.2 uni membranes). Therapeutic compositions generally are placed into a container having a sterile access port, for example, an intravenous solution bag or vial having a stopper pierceable by a hypodermic injection needle. The therapies based on a DNA-targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The synthetic circular guide RNA described herein, along with a desired gene editing system components, can be delivered to a cell of interest by various delivery systems such as vectors, e.g., plasmids and delivery vectors. In some aspects, circular mRNA is delivered by said delivery systems as described herein.

The synthetic circular guide RNA and/or circular mRNA described herein can be delivered by nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 2 (below).

TABLE 2
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethyl- | DOTMA | Cationic |
| ammonium chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis | GAP- | Cationic |
| (dodecyloxy)-1-propanaminium bromide | DLRIE | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethyl- | 2Oc | Cationic |
| pyridinium | | |
| 2,3-Dioleyloxy-N-[2-sperminecarboxamido- | DOSPA | Cationic |
| ethyl]-N,N-dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis | MDRIE | Cationic |
| (tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)-carbamoyl] | DC-Chol | Cationic |
| cholesterol | | |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[2,3-Dioctadecyloxypropyl)(2-hydroxyethyl)]- | CLIP-1 | Cationic |
| dimethylammonium chloride | | |
| rac-[2-(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl-aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylamino- | diC14- | Cationic |
| propionamidine | amidine | |
| Octadecenolyoxy[ethyl-2-heptadecenyl-3 | DOTIM | Cationic |
| hydroxyethyl]imidazolinium chloride | | |
| N1-Cholesteryloxycarbonyl-3,7-diazanonane- | CDAN | Cationic |
| 1,9-diamine | | |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilionleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 3 lists exemplary polymers for use in gene transfer and/or nanoparticle formulations.

TABLE 3
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 4 summarizes delivery methods for a polynucleotide encoding a Cas9 described herein.

TABLE 4

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system including the synthetic circular gRNA describe herein may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33 (1): 73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive the CRISPR system (e.g., including the synthetic gRNA described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, separate promoters drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas9 and synthetic circular gRNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a circular guide RNA or a Cas9-encoding mRNA, can be delivered in the form of RNA. Cas9 encoding mRNA can be generated using in vitro transcription. For example, Cas9 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas9 sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising gene editing system (e.g., including the synthetic circular gRNA and/or circular mRNA, described herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et ah, 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas9 described herein) are provided as part of a pharmaceutical composition. In some embodiments, the pharmaceutical composition comprises any of the fusion proteins provided herein (e.g., including the nucleobase editor described herein comprising LubCas9). In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. In some embodiments pharmaceutical composition comprises a circular messenger RNA. Pharmaceutical compositions can optionally comprise one or more additional therapeutically active substances.

### Kits

In one aspect, the synthetic circular gRNA described herein can be provided and or produced by a kit containing any one or more of the elements disclosed in the above methods and compositions. For example, a kit may include a cgRNA, a ligase, and suitable buffering reagents.

In some embodiments, the kit further comprises a nucleobase editor.

In some aspects, the kit comprises a synthetic circular mRNA. In some embodiments, the kit comprises a circular mRNA and a gene editing system. For example, a kit may include one or more of a circular mRNA, circular guide RNA, a ligase, suitable buffering agents and/or a nucleobase editor.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1: Ligation-Based Approach to Synthesis of Circular Guide RNA

This example describes the use of enzymatic ligation to join the ends of synthetically derived RNA, for example, by solid-phase synthesis (SPS) or enzymatically derived RNA, for example, by T7 RNA polymerase. Two ligation methods were tested that employ linear synthetic RNA modified with a terminal 5′ phosphate.

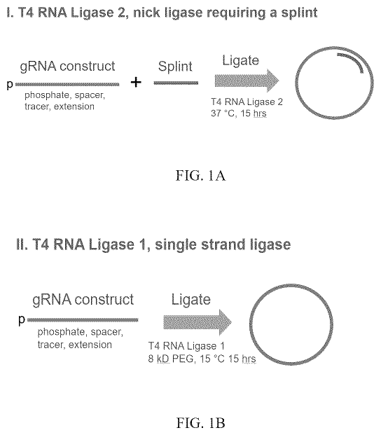

In some embodiments, an exemplary ligation method using T4 RNA ligase 2 is employed, which uses short DNA splints to pre-organize the ends of linear gRNA for ligation by a nick-joining ligase (e.g. T4 RNA ligase 2) (FIG. 1A). In some other embodiments, an exemplary ligation method is employed using a single stranded RNA ligase, T4 RNA ligase 1 to join the untemplated ends of RNA (FIG. 1B).

A comparison between the two methods was carried out to determine the highest yield of circular guide RNA (cgRNA) synthesis for SpCas9. Six Cas9 gRNAs were synthesized by solid phase synthesis (SPS) with different linker lengths (10-20 nucleotides; 110-120 nucleotides total).

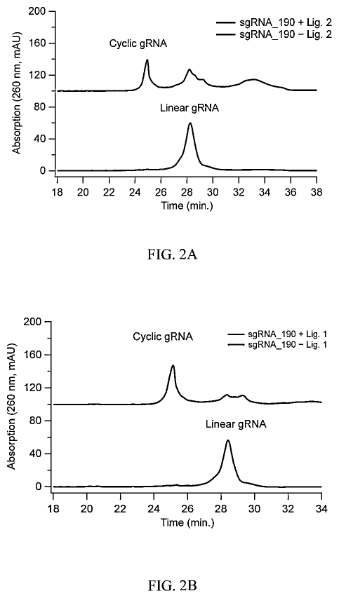

The amount of cyclic gRNA in the presence of T4 RNA ligase 2 and linear gRNA in control samples that were not treated with T4 RNA ligase 2 was quantified by measuring absorption at 260 nm (FIG. 2A). Only two gRNAs were cyclized with a yield of less than 50% with T4 RNA ligase 2.

The amount of cyclic gRNA in the presence of T4 RNA ligase 1 and linear gRNA in control samples that were not treated with T4 RNA ligase 1 was quantified by measuring absorption at 260 nm (FIG. 2B).

Conversion to cyclic gRNA was found to be higher using the T4 RNA Ligase 1 method (FIG. 2B). All six gRNAs tested were cyclized with a yield of 67-83% (FIG. 2B). Thus T4 RNA ligase 1 resulted in optimal yield of cgRNA.

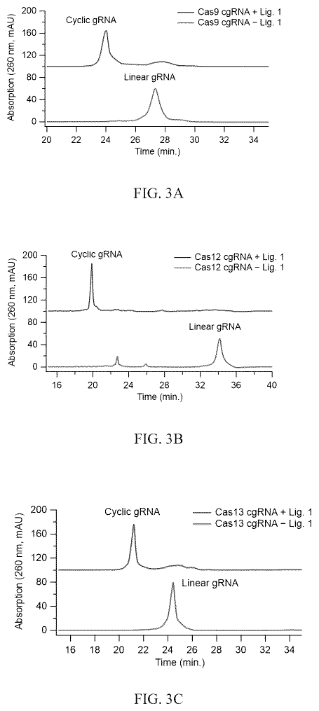

Using the T4 RNA ligase 1 method, eight cgRNAs for Cas12a and six cgRNAs for Cas13 were synthesized. The amount of cyclic gRNA was measured for each of these Cas gRNAs (FIG. 3A-3C).

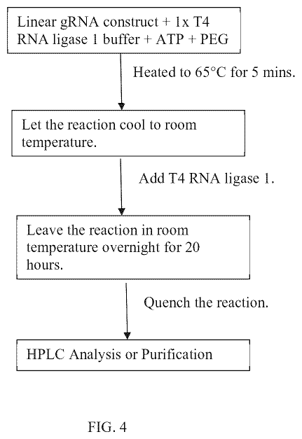

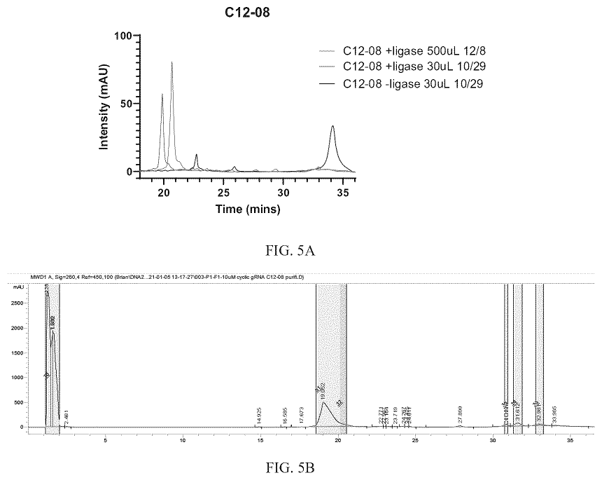

A schematic for generation of cgRNA by enzymatic ligation is shown in FIG. 4.

Table 5 provides the sequences for cgRNAs generated for Cas12a, Cas13 and Cas9.

TABLE 5
Circular guide RNA sequences for Cas12a, Cas13 and Cas9.

| RNA Name | Sequence | Cas enzyme, Notes |
|---|---|---|
| C12-01 | AAUUUCUACUCUUGUAGAUCUAGGAAUAUUGAAG | Cas12a, Control. |
| | GGGGCAGG (SEQ ID NO: 3) | No modifications. |
| C12-02 | mAsmAsmUsUUCUACUCUUGUAGAUCUAGGAAUA | Cas12a, Control. |
| | UUGAAGGGGGmCsmAsmGsG (SEQ ID NO: 4) | End modifications |
| C12-03 | PAAUUUCUACUCUUGUAGAUCUAGGAAUAUUGAA | Cas12a, No linker. |
| | GGGGGCAGG (SEQ ID NO: 5) | |
| C12-04 | pAAUUUCUACUCUUGUAGAUCUAGGAAUAUUGAA | Cas12a, Dimer. No |
| | GGGGGCAGGAAUUUCUACUCUUGUAGAUCUAGGA | linker. |
| | AUAUUGAAGGGGGCAGG (SEQ ID NO: 6) | |
| C12-05 | pAACUUUAAAUAAUUUCUACUCUUGUAGAUCUAG | Cas12a, 10 nt linker |
| | GAAUAUUGAAGGGGGCAGG (SEQ ID NO: 7) | |
| C12-06 | pAACUUUAAAUAACUUUAAAUAAUUUCUACUCUU | Cas12a, 20 nt linker |
| | GUAGAUCUAGGAAUAUUGAAGGGGGCAGG (SEQ | |
| | ID NO: 8) | |
| C12-07 | pAACUUUAAAUAACUUUAAAUAACUUUAAAUAAU | Cas12a, 30 nt linker |
| | UUCUACUCUUGUAGAUCUAGGAAUAUUGAAGGGG | |
| | GCAGG (SEQ ID NO: 9) | |
| C12-08 | pAAUUUCUACUCUUGUAGAUAAUUUCUACUCUUG | Cas12a, Direct |
| | UAGAUCUAGGAAUAUUGAAGGGGGCAGG (SEQ | repeat (19 nt) as |
| | ID NO: 10) | linker |
| C13-01 | PUCAAGGCACUCUUGCCCACGCCACCAGCUCCAA | Cas13 |
| | CUACCACAAGUUUAUAUGUUGUGGAAGGUCCAGU | |
| | UUUGGGGGCUAUUACAACAUUmC*mG*mA*U | |
| | (SEQ ID NO: 11) | |
| C13-02 | PUCAAGGCACUCUUGCCCACGCCACCAGCUCCAA | Cas13 |
| | CUACCACAAGUUUAUAUGUUGUGGAAGGUCCAGU | |
| | UUUGGGGGCUAUUACAACAUUCGAU (SEQ ID | |
| | NO: 12) | |
| C13-03 | PUCAAGGCACUCUUGCCCACGCCACCAGCUCCAA | Cas13 |
| | CUACCACAAGUUUAUAUGUUGUGGAAGGUCCAGU | |
| | UUUGGGGGCUAUUACAACAUUmC*mG*mA*UUCA | |
| | AGACGAUCGCAU (SEQ ID NO: 13) | |
| C13-04 | PUCAAGGCACUCUUGCCCACGCCACCAGCUCCAA | Cas13 |
| | CUACCACAAGUUUAUAUGUUGUGGAAGGUCCAGU | |
| | UUUGGGGGCUAUUACAACAUUCGAUUCAAGACGA | |
| | UCGCAU (SEQ ID NO: 14) | |
| C13-05 | PUCAAGGCACUCUUGCCCACGCCACCAGCUCCAA | Cas13 |
| | CUACCACAAGUUUAUAUGUUGUGGAAGGUCCAGU | |
| | UUUGGGGGCUAUUACAACAUUmC*mG*mA*UUCA | |
| | AGACGAUCGCAUUUGCGCCAUCUUAUCCAAGAAG | |
| | UACUGUAU (SEQ ID NO: 15) | |
| C13-06 | PUCAAGGCACUCUUGCCCACGCCACCAGCUCCAA | Cas13 |
| | CUACCACAAGUUUAUAUGUUGUGGAAGGUCCAGU | |
| | UUUGGGGGCUAUUACAACAUUCGAUUCAAGACGA | |
| | UCGCAUUUGCGCCAUCUUAUCCAAGAAGUACUGU | |
| | AU (SEQ ID NO: 16) | |
| C9-01 | pAUCGACAAGAAAGGGACUGAGUUUUAGAGCUAG | Cas9 |
| | AAAUAGCAAGUUAAAAUAAGGCUAGUCCGUUAUC | |
| | AACUUGAAAAAGUGGCACCGAGUCGGUGCUUUUU | |
| | UUUUUUUUU (SEQ ID NO: 17) | |
| C9-02 | pAUCGACAAGAAAGGGACUGAGUUUUAGAGCUAG | Cas9 |
| | AAAUAGCAAGUUAAAAUAAGGCUAGUCCGUUAUC | |
| | AACUUGAAAAAGUGGCACCGAGUCGGUGCUUUUU | |
| | UUUUUUUUUUUUUU (SEQ ID NO: 18) | |
| C9-03 | pAUCGACAAGAAAGGGACUGAGUUUUAGAGCUAG | Cas9 |
| | AAAUAGCAAGUUAAAAUAAGGCUAGUCCGUUAUC | |
| | AACUUGAAAAAGUGGCACCGAGUCGGUGCUUUUU | |
| | UUUUUUUUUUUUUUUUUUU (SEQ ID NO: | |
| | 19) | |
| C9-04 | pACCAUCGACAAGAAAGGGACUGAGUUUUAGAGC | Cas9 |
| | UAGAAAUAGCAAGUUAAAAUAAGGCUAGUCCGUU | |
| | AUCAACUUGAAAAAGUGGCACCGAGUCGGUGCUU | |
| | UUUUUUUUUUUUUUUUU (SEQ ID NO: 20) | |
| C9-05 | pAUCGACAAGAAAGGGACUGAGUUUUAGAGCUAG | Cas9 |
| | AAAUAGCAAGUUAAAAUAAGGCUAGUCCGUUAUC | |
| | AACUUGAAAAAGUGGCACCGAGUCGGUGCUUUUA | |
| | UCGCGUGCUCUCGU (SEQ ID NO: 21) | |
| C9-06 | PACCAUCGACAAGAAAGGGACUGAGUUUUAGAGC | Cas9 |
| | UAGAAAUAGCAAGUUAAAAUAAGGCUAGUCCGUU | |
| | AUCAACUUGAAAAAGUGGCACCGAGUCGGUGCUU | |
| | UUAUCGCGUGCUCUCGU (SEQ ID NO: 22) | |

### Example 2: Purification of cgRNA Synthesized by Enzymatic Ligation

In some embodiments, circular guide RNAs synthesized by enzymatic ligation were purified, desalted and analyzed by HPLC.

Briefly, T4 RNA ligase 1 was used to generate circular gRNAs for Cas12a, Cas9 and Cas13. The reaction was scaled up from 30 μl to 500 μl. Purified cyclic gRNA was then analyzed via anion exchange chromatography.

The Cas12a gRNA analysis gradient is shown in FIG. 5A. The Cas12a gRNA purification profile is shown in FIG. 5B.

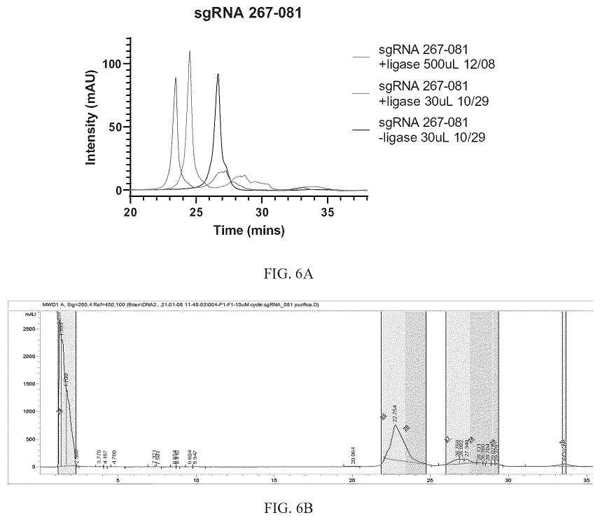

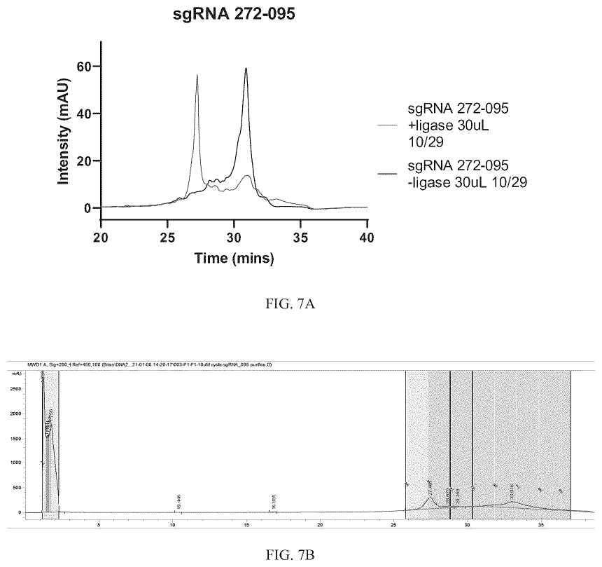

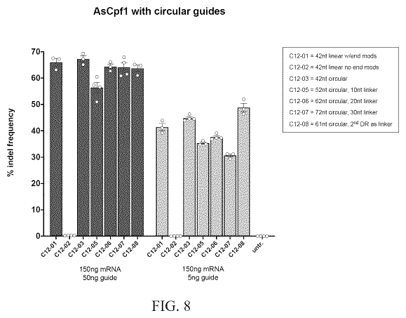

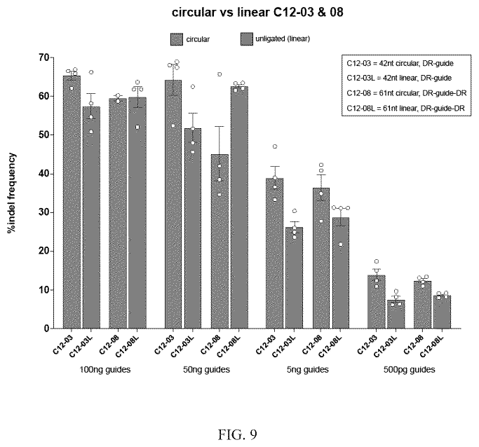

The Cas13 gRNA analysis gradient is shown in FIG. 6A. The Cas13 gRNA purification profile is shown in FIG. 6B.

The Cas9 gRNA analysis gradient is shown in FIG. 7A. The Cas9 gRNA purification profile is shown in FIG. 7B.

HPLC gradient is shown in Table 6.

TABLE 6
HPLC gradient

| Time | Elution |
|---|---|
| (mins.) | buffer (%) |
| 0 | 65 |
| 30 | 100 |
| 38 | 100 |
| 39 | 65 |
| 47 | 65 |

Circular gRNAs were generated and purified with high yield for Cas12a, Cas13 and Cas9.

### Example 3: Gene Editing Efficiency with Cas12a Circular Guide RNA

In this example, editing of cgRNAs generated for Cas12a were tested at two different concentrations of RNA, at a saturating concentration of gRNA of 50 ng and at a limiting concentration of gRNA of 5 ng. Percentage indel frequency was measured to determine gene editing efficiency (FIG. 8).

Three 42 nt linear guide RNAs were tested in the absence of any linker sequence for Cas12 (AsCpf1)—a linear gRNA without end modification, a linear gRNA with end modification, and a circular gRNA. A linear gRNA without end modification, C12-02, showed no editing due to degradation at both concentrations of guide RNA used. In contrast, a linear guide RNA with end modification to prevent degradation, C12-01, resulted in about 40% indel frequency at limiting concentrations of guide RNA and up to 65% indel frequency at saturating concentrations of guide RNA. Comparable editing was observed with circular gRNA, C12-03.

Cas12 administered with a 52 nt circular gRNA comprising a 10 nt linker (C12-05) resulted in about 55% editing frequency at a concentration of 50 ng gRNA and about 35% editing frequency at a concentration of 5 ng gRNA. Cas12 administered with a 62 nt circular gRNA comprising a 20 nt linker (C12-06) resulted in about 65% editing frequency at a concentration of 50 ng gRNA and about 35% editing frequency at a concentration of 5 ng gRNA. Cas12 administered with a 72 nt circular gRNA comprising a 30 nt linker (C12-07) resulted in about 65% editing frequency at a concentration of 50 ng gRNA and about 30% editing frequency at a concentration of 5 ng gRNA. Cas12 administered with a 61 nt circular gRNA comprising a second direct repeat as a linker (C12-08) resulted in about 65% editing frequency at a concentration of 50 ng gRNA and about 45% editing frequency at a concentration of 5 ng gRNA.

In summary, all the cgRNAs tested showed activity that was comparable to end modified gRNA, while unmodified linear gRNA showed no editing. Unlike for Cas9, a linker was not found to be required for Cas12 (C12-03). Linkers are likely not required for Cas12 or Cas13 to function due to activity of their own catalytic domains for RNA processing. Since including the direct repeat sequence (as found in the natural CRISPR system) as the linker element significantly increased activity above end modified gRNA, this demonstrated a method for engineering Cas12 circular guides with high activity.

### Example 4: Effect of Direct Repeats in Gene Editing Efficiency with Cas12a Circular or Linear Guide RNA

In this example, two exemplary circular Cas12a gRNAs were tested for gene editing efficiency at different concentrations of guide RNA, starting at 500 μg, 5 ng, 50 ng and 100 ng. Percentage indel frequency was measured to determine gene editing efficiency (FIG. 9).

Comparative results are shown for linear and circular Cas12a gRNAs, each of 42 nt length and 61 nt length. Both linear and circular Cas12a gRNAs comprised direct repeat sequences. The 42 nt guide RNA, C12-03 comprised a direct repeat sequence at the 5′ end. The 61 nt guide RNA, C12-08 comprised two direct repeat sequences at both the 5′ and 3′ end of the guide RNA.

The results showed that indel frequency increased with increasing concentration of gRNAs. About 60-65% indel frequency was achieved at a concentration of 100 ng circular guide RNA relative to about 10-15% at 500 pg guide RNA concentration. For both 42 nt and 61 nt guide RNAs, indel frequency was higher in the case of circular gRNAs relative to linear gRNAs (FIG. 9).

Overall, including the direct repeat sequence (as found in the natural CRISPR system) as the linker element significantly increased editing activity of circular guide RNAs to greater than activity of linear gRNAs comprising the same direct repeat sequence.

### Example 5: Self-Circularizing RNAs Using Twister Ribozymes

In this example, guide RNAs are circularized using a circularizing technique that employs Twister ribozymes. In this method, twister ribozymes are used to generate specific ends (5′ hydroxyl and 2′-3′ cyclic phosphate) in an RNA transcript in cis. Following this, the ends are ligated by RtcB, a ubiquitous and endogenous RNA ligase. The self-circularizing guides are delivered as DNA plasmids driven by U6 promoter, where both cleavage and ligation steps take place directly in transfected cells, or transcribed in vitro using a T7 promoter, then transfected following in vitro ligation with purified RtcB.

In this example, a U6/T7 hybrid promoter was used. The F30 arms and U6 terminator were not used. Guides were made in vitro but transfected into HEK293T cells and allowed to ligate in cells.

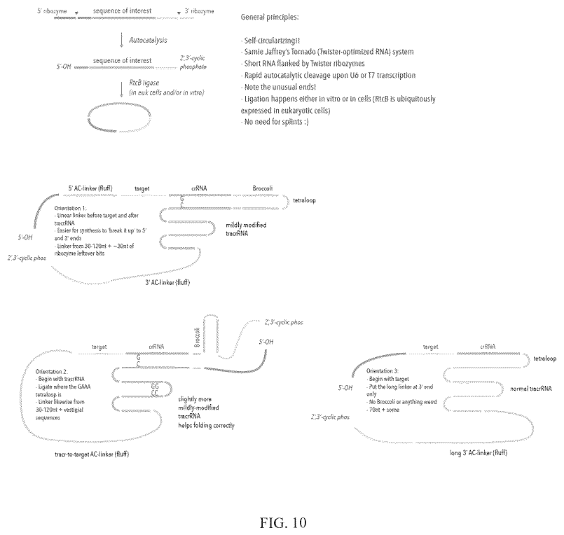

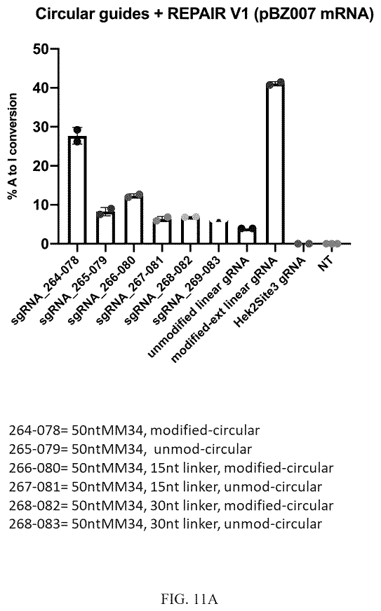

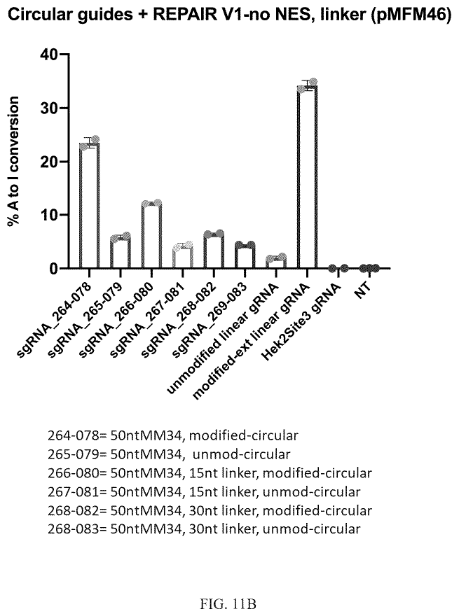

In this example, the sgRNA was connected to the ribozyme ends in one of three architectures (FIG. 10). Each of the architectures uses a linker sequence (exemplary repeating AC motifs are used herein to aid linear folding and avoid non-specific interactions with sgRNA scaffold). The linker lengths are optimized so that the circularized guide is long enough to not interfere with Cas9 loading and activity. The constructs made for testing had linker lengths of 45-115 nt and included some cgRNAs with linker only at the 3′ end and some with linker at both 5′ and 3′ ends. Some of the guides include an optional broccoli aptamer attached, which are for diagnostic purposes only and serve no function in Cas9 cleavage/binding of targets.

In orientation 1, the linear linker is connected 5′ of the target, followed by crRNA, broccoli marker, tracrRNA and then a linear linker at the 3′ of the tracrRNA. A linker from 30-120 nt is used and includes about 30 nt additional sequences. Ligation occurs joining linkers at 5′ OH and 2′3 cyclic phosphate ends producing cgRNA. In orientation 2, the linker begins with a modified tracrRNA which helps proper folding and ligation occurs at the GAAA tetraloop connecting the tracrRNA at the 5′ end to broccoli. The linker likewise comprises about 30-120 nt and some additional vestigial sequences and joins the tracrRNA at the 3′ end to the target sequence, followed by crRNA. In orientation 3, the target is followed by crRNA, tracrRNA and a long linker at the 3′ end of the tracrRNA only. This architecture does not include broccoli and is about 70 nt long and comprises some additional vestigial sequences.

Using this approach, circular gRNAs for Cas9 have been generated using the twister ribozyme system in cells. Circular gRNAs were synthesized for exemplary sequence with 100, 110, 120 and 150 nt guide RNA lengths. The guides were synthesized using standard solid phase synthesis chemistry and enzymatically ligated. The 100 and 120 nt guide RNAs did not show editing, but longer guide RNAs are being tested. Similarly, circular guides for Cas12b were also generated. These are cleaved by Cas12b RNAse function in cells and carry out gene editing while in linear form within cells. Cas13 guide RNAs have also been cyclized and these will linearize in cells prior to gene editing.

TABLE 7
Exemplary cgRNA tested in HEK293T cells are shown.

| | | | Max | Max |
|---|---|---|---|---|
| | | | A to G | A to G |
| CYL_Editor | CYL_Editor_Desc | FAR_target | rate | rate |
| DNA460 | HEK2 normal with | Hek2 | 29.196 | 19.625 |
| | Broccoli | | | |
| DNA461 | HEK2 AC40-30 | Hek2 | 0.047 | 0.097 |
| | orientation 1 | | | |
| DNA462 | HEK2 ext00-30 | Hek2 | 0.078 | 0.031 |
| | orientation 1 | | | |
| DNA463 | HEK2 ext30-30 | Hek2 | 0.043 | 0.039 |
| | orientation 1 | | | |
| DNA464 | HEK2 ext60-30 | Hek2 | 0.087 | 0.051 |
| | orientation 1 | | | |
| DNA465 | HEK2 AC40-30 | Hek2 | 0.175 | 0.127 |
| | orientation 1 no broc | | | |
| DNA466 | HEK2 AC40-30 | Hek2 | 0.104 | 0.103 |
| | orientation 1 no broc | | | |
| | tracr2 | | | |
| DNA467 | HEK2 AC30 | Hek2 | 0.026 | 0.029 |
| | orientation 2 tracr2 | | | |
| DNA468 | HEK2 AC60 | Hek2 | 0.644 | 0.37 |
| | orientation 2 tracr2 | | | |
| DNA469 | HEK2 AC90 | Hek2 | 0.032 | 0.03 |
| | orientation 2 tracr2 | | | |
| DNA470 | HEK2 AC105-ext20 | Hek2 | 0.13 | 0.09 |
| | orientation 2 tracr2 | | | |
| DNA471 | HEK2 AC60-ext20 | Hek2 | 0.029 | 0.032 |
| | orientation 2 no broc | | | |
| | tracr2 | | | |
| DNA472 | HEK2 AC60-ext20 | Hek2 | 0.033 | 0.033 |
| | orientation 2 no broc | | | |
| | tracr1 | | | |
| DNA473 | HEK2 AC70 | Hek2 | 7.383 | 4.982 |
| | orientation 3 natural | | | |
| DNA474 | HEK2 tracr1_T7 | Hek2 | 0.318 | 0.126 |
| | control no fluff | | | |
| DNA475 | HEK2 tracr2_T7 | Hek2 | 0.264 | 0.139 |
| | control no fluff | | | |
| DNA476 | HEK2 tracr1_U6 only | Hek2 | 28.807 | 19.217 |
| DNA477 | HEK2 tracr2_U6 only | Hek2 | 32.346 | 18.607 |
| DNA478 | HEK2 EFS ext20 | Hek2 | 0.034 | 0.033 |
| | tracr1 | | | |
| DNA479 | HEK2 EFS ext2 tracr1 | Hek2 | 0.291 | 0.095 |
| DNA480 | HEK2 normal | Hek2 | 34.207 | 21.089 |
| None | HEK2 normal | Hek2 | 0.031 | 0.048 |
| None | HEK2 normal | Hek2 | 0.039 | 0.027 |
| None | HEK2 normal | Hek2 | 0.02 | 0.03 |

The results showed that an exemplary cgRNA with a 70-nt AC linker in architecture 3 was functional. The sequence of the cgRNA is provided in Table 8.

TABLE 8
Sequence of exemplary cgRNA synthesized using Twister ribozyme

A cgRNA synthesized using Twister ribozyme
with 70 nt AC linker (Architecture 3)
GGCCGCACTCGCCGGTCCCAAGCCCGGATAAAATGGGAGGGGGCGGGAAACCGCCTAACCA
TGCCGAGTatgcatGGAACACAAAGCATAGACTGCGTTTTAGAGCTAGAAATAGCAAGTTA
AAATAAGGCTAGTCCGTTATCAACTTGAAAAAGTGGCACCGAGTCGGTGCTTTAAAACAAC
AACCCAACCAACAAACCAACACCAAACAACAAACCAAACCCCAACAAACAACCACCAACCC
AgaattcTCGGCGTGGACTGTAGAACACTGCCAATGCCGGTCCCAAGCCCGGATAAAAGTG
GAGGGTACAGTCCACGC (SEQ ID NO: 23)
Ribozyme sequences flanking gRNA (underlined)
Arbitrary linker sequence (italics)
Restriction sites (bold)
Guide RNA (bold, underlined)
Normal SpCas9 sgRNA scaffold (bold, italics, underlined)

### Example 6: RNA Editing Using cgRNAs

This example shows the successful editing of RNA using cgRNAs.

For these studies, 40,000 HEK293T cells were seeded in 48 well plates. The cells were cultured for 24 hours, followed by transfection of gRNA (either circular or linear) with REPAIR base editor using 1 μl of Messenger Max™. Forty eight hours post-transfection, the cells were harvested and RNA was extracted. RNA extraction was performed using QIAGEN 94-well kit, followed by cDNA with Random hexamer primer. Next generation sequencing was then performed.

The REPAIR editors used in these studies perform A to G edits only. Two kinds of REPAIR editors were used: 1) REPAIR editor V1 (has no extra linkers and contains nuclear export sequence (NES) sequence); and 2) REPAIR editor V1-B (has an extra linker and no NES).

The ability of cgRNA using REPAIR VI editor to target RNA editing on KRAS was tested. The data from these studies showed that cgRNA guides can direct on target RNA editing on KRAS (FIG. 11A). The data from these studies further showed that circular unmodified gRNAs had less efficiency compared to circular and linear modified gRNAs.

The ability of cgRNA using REPAIR V1-B editor to target RNA editing on KRAS was also tested (FIG. 11B). The data from these studies showed that using an alternative REPAIR editor did not improve editing efficiency when using circular or linear gRNAs. When compared to REPAIR VI editor, a similar pattern of efficiency was observed among circular and linear gRNAs.

Collectively, the data from these studies show the successful editing of RNA using cgRNAs.

### Example 7: Ligation-Based Approach to Synthesis of Circular mRNA

In some embodiments, messenger RNA is used to synthesize circular RNA. This example describes the use of enzymatic ligation to join the ends of messenger RNA derived, for example, by in vitro transcription, for example, by T7 RNA polymerase. Ligation methods using T4 RNA ligase 1 and T4 RNA ligase 2 were tested that employ linear messenger RNA modified with a terminal 5′ phosphate.

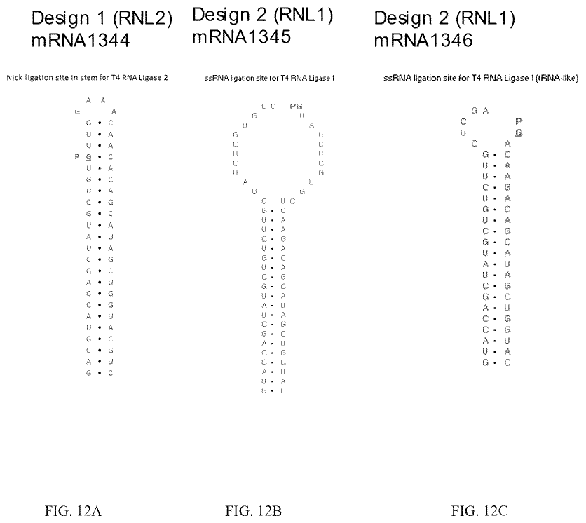

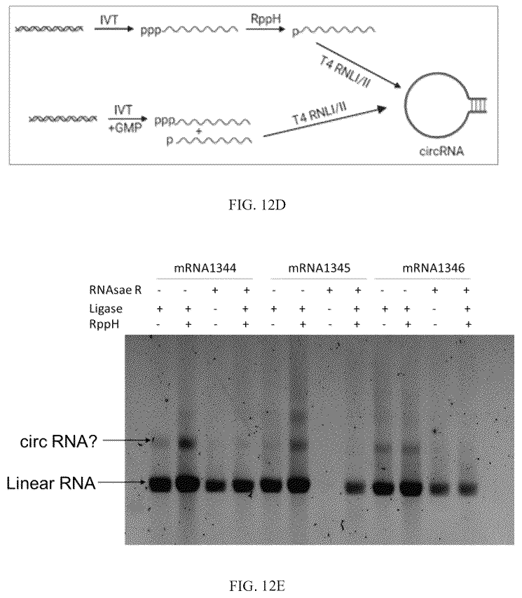

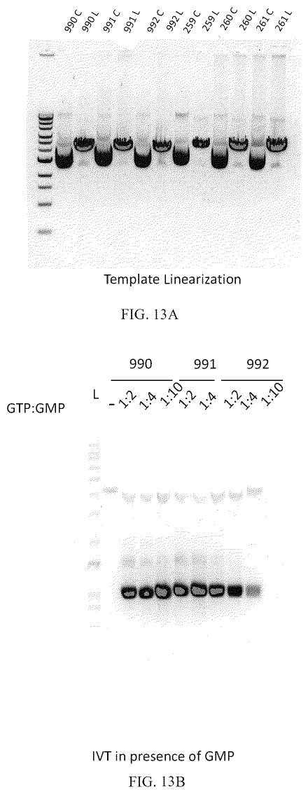

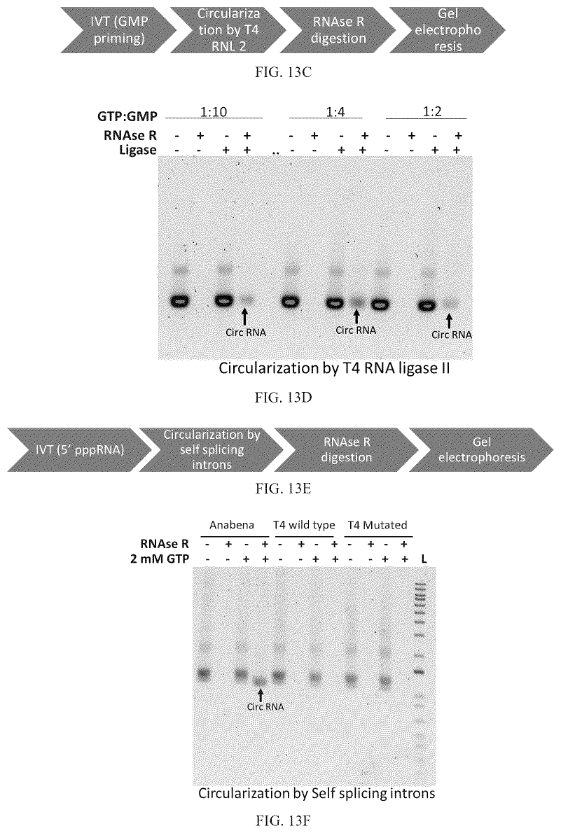

In some embodiments, an exemplary ligation method using T4 RNA ligase 2 is employed, which uses short DNA splints to pre-organize the ends of linear gRNA for ligation by a nick-joining ligase (e.g. T4 RNA ligase 2). For example, in some embodiments, the messenger RNA has a nick ligation site in the stem for the activity of T4 RNA ligase 2 (FIG. 12A). In some other embodiments, an exemplary ligation method is employed using a single stranded RNA ligase, T4 RNA ligase 1 to join the untemplated ends of RNA. For example, in some embodiments, the messenger RNA has a single-stranded RNA ligation site (FIG. 12B

[symbol]

In some embodiments, RNA generated by in vitro transcription that triphosphorylated at the 5′ terminal end is treated with RNA pyrophosphohydrolase (RppH) enzyme which cleaves diphosphate residues yielding mRNA with a terminal 5′ [symbol] other embodiments, in vitro transcription carrier [symbol] carrier [symbol] in the [symbol] phosphate (GMP) resulting in mRNA product [symbol] a mixture of 5′ triphosphorylated mRNA and 5′ monophosphorylated mRNA. In some other embodiments, heat annealing is carried out.

The monophosphorylated mRNA, when treated with T4 RNA ligase 1 or T4 RNA ligase 2, circularized the mRNA (FIG. 12D).

The mRNA was analyzed by polyacrylamide gel electrophoresis (PAGE) carried out in the presence of a denaturing agent (FIG. 12E). Gel analysis was conducted on samples treated with T4 RNA ligase 1 or T4 RNA ligase 2, RNA pyrophosphohydrolase (RppH) and/or RNAse R. The results showed circularized mRNA in a slower migrating band on the PAGE gel than the linear mRNA. Circular mRNA was most abundant in samples treated with both RppH and ligase enzymes. Appreciable amounts of circular mRNA were also observed in samples treated only with ligase enzyme.

Overall, the data from these studies showed successful circularization of messenger RNA using T4 RNA ligase 1 and T4 RNA ligase 2.

### Example 8: In Vitro Transcription in the Presence of Guanosine Monophosphate (GMP) Produced 5′ Phosphorylated mRNA

Exemplary template RNAs were linearized and linear and control (before linearization) forms were run on a gel. As shown in FIG. 13A, the linear forms were slower migrating than corresponding non-linearized or supercoiled forms. The results showed that the templates were substantially linearized and very faint bands corresponding to non-linearized or supercoiled form was seen in the linearized fraction (FIG. 13A).

In vitro transcription was carried out on linearized templates using 7.5 mM NTP, 5 mM guanosine triphosphate (GTP) and either 10 mM, 20 mM or 50 mM guanosine monophosphate (GMP), resulting in mRNA product that is a mixture of 5′ triphosphorylated mRNA and 5′ monophosphorylated mRNA. As shown in FIG. 13B, the results showed that at a ratio of 1:2, 1:4 and 1:10 guanosine triphosphate to guanosine monophosphate ratio (GTP:GMP), 5′-phosphorylated mRNA was produced.

Overall, the results showed that 5′-phosphorylated mRNA was produced with guanosine monophosphate priming.

### Example 9: Circularization of mRNA is Carried Out by Enzymatic Ligation or by Self-Splicing Introns

This example describes circularization of mRNA by two methods-enzymatic ligation or self-splicing introns. As shown in FIG. 13C, in some embodiments, circularization is carried out by an enzymatic method which comprises the steps of in vitro transcription with guanosine monophosphate priming, followed by circularization using T4 RNA ligase 2, and subsequent RNase R digestion and analysis by gel electrophoresis.

T4 RNA ligase 2 was used to circularize three exemplary mRNAs-2618, 2619 and 2620 synthesized by in vitro transcription in the presence of guanosine monophosphate priming at a guanosine triphosphate to guanosine monophosphate ratio (GTP:GMP) of 1:2, 1:4 or 1:10.

The results are shown in FIG. 13D. In the absence of RNAse and ligase, slower migrating and faster migrating RNA species were observed, with the faster migrating species being predominant (lane 1, 6 and 10). In the presence of RNAse R, both slower and faster migrating RNA species were entirely digested by RNAse R, indicating that these were linear RNA species (lane 2, 7 and 11). In the presence of ligase enzyme, a slower migrating and a faster migrating form of RNA were seen, similar to the lane without ligase. The faster migrating RNA species was predominant (lane 3, 8 and 12). In the presence of RNAse R, the linear RNA species were digested but an RNA species was seen co-migrating with the faster moving linear species, indicating that it was an RNAse resistant circularized RNA (lane 4, 9 and 13). Lane 5 is empty. The results showed that RNAs synthesized in all three guanosine triphosphate to guanosine monophosphate ratio (GTP:GMP) ratios showed a band resistant to RNAse R indicating circularization of RNA.

As shown in FIG. 13E, in some embodiments, circularization is carried out by a self-splicing mechanism which comprises the steps of in vitro transcription in the presence of guanosine monophosphate generating RNA with 5′ triphosphate, circularization by self-splicing introns, and subsequent RNase digestion with RNAse R and/or 2 mM GTP, followed by gel electrophoresis. In some embodiments, a self-splicing intron using permuted group I catalytic intron is used to circularize RNA. In some embodiments, GTP and Mg2+ are used as cofactors. Three different self-splicing introns (Anabaena, WT T4 and mutated T4) were designed for template circularization. Briefly, circular RNA is synthesized in vitro in which transposed halves of a split group I intron flank the sequence of the RNA to be circularized. Exemplary permuted intron-exon (PIE) sequences for self-splicing introns are as shown in Table 9. (Wesselhoeft, R A et al., Nature Communications. 2018. 9:2629, p. 1-10; Rausch et al., Nucleic Acids Research. 2021. 49 (6). p. 1-13). The permuted intron-exon splicing strategy comprises fused partial exons flanked by half-intron sequences which undergo double transesterification reactions characteristic of group I catalytic introns, but because the exons are fused they are excised as covalently linked circular RNA from 5′ to 3′, thus yielding circular RNA.

TABLE 9
Exemplary permuted intron-exon (PIE) sequences for self-splicing introns
Intron of tRNA Leu in Anabaena (cyanobacteria)
CCGTCGATTGTCCACTGGTCAACAATAGATGACTTACAACTAATCGGAAGGTGCAGAGACT
CGACGGGAGCTACCCTAACGTCAAGACGAGGGTAAAGAGAGAGTCCAATTCTCAAAGCCAA
TAGGCAGTAGCGAAAGCTGCAAGAGAATGaaaaTCCGTTGACCTTAAACGGTCGTGTGGGT
TCAAGTCCCTCCACCCCCACGCCGGAAACGCAATAGCCGAAAAACAAAAAACAAAAAAACC
(SEQ ID NO: 24)
AAAAAACAAAAAACAAAACGGCTATTATGCGTTACCGGCGAGACGCTACGGACTTaaataa
ttGAGCCTTAAAGAAGAAATTCTTTAAGTGGATGCTCTCAAACTCAGGGAAACCTAAATCT
AGTTATAGACAAGGCAATCCTGAGCCAAGCCGAAGTAGTAATTAGTAAGACCAGTGGACAA
TCGACGG (SEQ ID NO: 25)
Td (thymidylate synthase) intron of T4 bacteriophage
CCTTTGGGGAGTAGGGTCAAGTGACTCGAAACGATAGACAACTTGCTTTAACAAGTTGGAG
ATATAGTCTGCTCTGCATGGTGACATGCAGCTGGATATAATTCCGGGGTAAGATTAACGAC
CTTATCTGAACATAATGCTACCGTTTAATATTGCGTCA (SEQ ID NO: 26)
CAGAGATGTTTTCTTGGGTTAATTGAGGCCTGAGTATAAGGTGACTTATACTTGTAATCTA
TCTAAACGGGGAACCTCTCTAGTAGACAATCCCGTGCTAAATTGTAGGACTA (SEQ ID
NO: 27)
Modified td intron sequences of T4 bacteriophage
GGGAGACCCTCGAATGGAATTGGTTCTACATAAATGCCTAACGACTATCCCTTTGGGGAGT
AGGGTCAAGTGACTCGAAACGATAGACAACTTGCTTTAACAAGTTGGAGATATAGTCTGCT
CTGCATGGTGACATGCAGCTGGATATAATTCCGGGGTAAGATTAACGACCTTATCTGAACA
TAATGct
(SEQ ID NO: 28)
TTGGGTTAATTGAGGCCTGAGTATAAGGTGACTTATACTTGTAATCTATCTAAACGGGGAA
CCTCTCTAGTAGACAATCCCGTGCTAAATTGTAGGACTAATTCCATTTATCAGATTTCTAG
A (SEQ ID NO: 29)

As shown in FIG. 13F, in the absence of 2 mM GTP and RNAse R, slower migrating and faster migrating RNA species were observed, with the faster migrating species being predominant (lanes 1, 5, 9). In the presence of RNAse R, both these species were digested, indicating that these were linear forms (lanes 2, 6, 10). In the presence of 2 mM GTP, two species of RNA were seen similar to what was observed in lane 1 (lanes 3, 7, 11). Gel analysis showed an RNAse R resistant band in RNA treated with Anabaena intron (lane 4) that migrated faster than the untreated control. No RNAse R resistant RNA species were detected in RNA samples treated with T4 intron (lanes 8 and 12). Overall, the results indicated circularization of RNA with Anabaena intron. A schematic of the mechanism of self-circularization by Anabaena intron is shown in FIG. 13G.

Without wishing to be bound by any particular theory, it is contemplated that Anabaena catalytic intron from Anabaena pre-tRNA results in a weakening of a short stretch of homology between the Internal Ribosome Entry Site (IRES) and the 3′ end of the coding region, which aided in the formation of an isolated splicing bubble. The Anabaena intron system used herein (SEQ ID NO: 24 and 25) strengthens this region of internal homology increasing splicing efficiency. The Anabaena intron also resulted in reduction in circRNA nicking compared to T4 intron. Without wishing to be bound by any particular theory, it is contemplated the Anabaena system successfully generated circular RNA (rather than T4 introns) due to increased splicing efficiency and generation of intact circular RNA without nicks.

The circularization of RNA was confirmed by RT-PCR using divergent primers as indicated in FIG. 13H. The RT-PCR product was visualized on a gel.

Table 10 shows the expected product size. The results showed the presence of expected band sizes after PCR, confirming that the circularization reaction was successful (FIG. 13I).

TABLE 10
Expected RT-PCR product size

| | | Expected | Expected |
|---|---|---|---|
| | | product | product |
| | | size | size |
| | | with | with |
| Lane | | primer | primer |
| No. | Condition | pair F1/R1 | pair F2/R2 |
| 1 | Circularization of T4 RNA ligase II, | 172 | 496 |
| | 1:2 GTP:GMP | | |
| 2 | Circularization of T4 RNA ligase II, | 172 | 496 |
| | GTP:GMP | | |
| 3 | Circularization of T4 RNA ligase II, | 172 | 496 |
| | 1:10 GTP:GMP | | |
| 4 | Anabaena | 270 | 594 |
| 5 | Td of bacteriophage T4 (WT) | 154 | 488 |
| 6 | Td of bacteriophage T4 (mod) | 130 | 454 |

Overall, the results from this example showed that circularization was achieved through both enzymatic methods as well as self-templating intron methods using, for example, Anabaena intron.

### Example 10: Cas12a (Cpf1) Editing with cgRNA in CD34⁺ Cells

This example describes the use of Cas12a (Cpf1) with circular guide RNA for base editing in CD34+ cells.

In this study, exemplary circular guide RNAs and their linear guide RNA counterparts were tested for gene editing in CD34+ cells. Exemplary guide RNAs, C12-03 (SEQ ID NO: 5, 42 nt, no linker), and C12-08 (SEQ ID NO: 10, 61 nt) which comprises a linker of conserved direct repeat (19 nt) were tested.

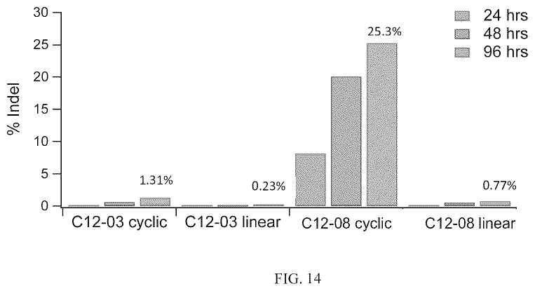

Circular or linear guide RNAs and Cas12a were introduced in CD34+ cells, and subsequently cells were harvested at 4 time points over 96 hours, i.e. 12 hours, 24 hours, 48 hours and 96 hours. The gene editing efficiency was quantified as % indel as shown in FIG. 14.

The results from this study showed that the C12-03 linear guide as well as the C12-03 circular guide, lacking a linker, showed less than 1% editing at all time-points in CD34+ cells.

Similarly, the C12-04 linear guide RNA showed <1% editing. However, the results showed that the C12-04 circular guide RNA showed significant editing: over 5% indel at 24 hours, about 20% indel at 48 hours and about 25% editing at 96 hours. Without wishing to be bound by any particular theory, it is contemplated that editing in CD34+ cells requires either end modification or circularization that minimizes or prevents gRNA degradation and enhances intracellular stability of gRNA and increases efficacy of genome editing.

Overall, the results from this study showed successful RNA editing in CD34+ cells using a circular guide RNA.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method of making circular guide RNA (cgRNA) comprising:
contacting a linear guide RNA with a ligating enzyme, wherein the contacting brings together a first end and a second end of the guide RNA, and wherein the ligating enzyme ligates the first and the second end of the guide R1′JA thus creating a cgRNA.

2. The method of claim 1, wherein (i) the linear guide RNA comprises a terminal 5′ phosphate; (ii) the ligating enzyme is selected from the group consisting of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, Splint® Ligase E. coli DNA Ligase, 9°N DNA Ligase, CircLigase, CircLigase II, DNA Ligase I, DNA Ligase III, and DNA Ligase IV, wherein the ligating enzyme is T4 RNA Ligase 2; (iii) the guide RNA is chemically synthesized or enzymatically synthesized; and/or (iv) the guide RNA further comprises a direct repeat sequence found in natural CRISPR systems.

3. (canceled)

4. The method of claim 1, wherein the ligating occurs in the absence of a template between the first end and the second end of the guide RNA, and wherein: (i) the first end and the second end of the guide RNA has partial complementarity of at least 5, 10, 15, or 20 nucleotides; and/or (ii) the ligating enzyme is a single-strand ligase, wherein the single-strand ligase is T4 RNA Ligase 1.

5.-8. (canceled)

9. The method of claim 1, wherein the ligating occurs in the presence of a template between the first end and the second end of the guide RNA, and wherein: (i) the template is an oligonucleotide splint, wherein the one or more oligonucleotide splints anneal to the first end and to the second end of the guide RNA thereby facilitating the formation of a loop structure between the first end and the second end of the guide RNA, further wherein the oligonucleotide splint is a DNA splint or an RNA splint having between 15 to 40 nucleotides; (ii) the oligonucleotide splint hybridizes with at least 5, 6, 7, 8, 9, 10, 11, 12, 13, 14, 15, 16, 17, 18, 19, 20, 21, 22, 23, 24, 25, 26, 27, 28, 29, or 30 nucleotides of the first end of the guide RNA; (iii) the oligonucleotide splint has perfect complementarity with 5, 6, 7, 8, 9, 10, 11, 12, 13, 14, 15, 16, 17, 18, 19, 20, 21, 22, 23, 24, 25, 26, 27, 28, 29, or 30 nucleotides of the first end of the guide RNA; (iv) the oligonucleotide splint hybridizes with at least two or more nucleotides of the second end of the guide RNA; and/or (v) the oligonucleotide splint has perfect complementarity with 2, 3, 4, 5, or six nucleotides of the second end of the guide RNA.

10.-20. (canceled)

21. The method of claim 1, wherein the guide RNA further comprises an RNA linker sequence positioned at the 5′ end and/or 3′ end of the guide RNA sequence or between the first end of the guide RNA and the second end of the guide RNA, and further wherein positioned at the 5′ end and/or 3′ end of the guide RNA sequence or between the first end of the guide RNA and the second end of the guide RNA, and further wherein: (i) the RNA linker sequence comprises between 1-50 nucleotides, 51-100 nucleotides, 100-150 nucleotides, or 150-200 nucleotides; (ii) the RNA linker sequence comprises 10 to 20 nucleotides; iii) the RNA linker comprises 20 to 30 nucleotides; iv) the RNA linker sequence comprises between 24 nucleotides and 200 nucleotides; v) wherein the linker sequence is placed only at the 3′ end; or vi) the 3′ end has a longer linker than the linker at the 5′ end, further optionally wherein the 3′ end of the guide RNA has a linker that is between 1-20 nucleotides, 5-50 nucleotides, 5-100 nucleotides longer than a linker at the 5′ end of the guide RNA.

22.-28. (canceled)

29. A method of making circular guide RNA (cgRNA) comprising:
modifying the ends of a linear guide RNA to create a first 5′ hydroxyl end and a second 2′-3′ cyclic phosphate end; and
ligating the first end and the second end with an RNA ligase thereby generating a cgRNA.

30. The method of claim 29, wherein:
(i) the ligating occurs in the absence of a template between the first end and the second end of the guide RNA;
(ii) the modifying the ends to create a first 5′ hydroxyl end and a second 2′-3′ cyclic phosphate end is performed by a ribozyme, wherein the ribozyme is a self-splicing ribozyme and/or wherein the ribozyme is a twister ribozyme;
(iii) the modifying the ends to create a first 5′ hydroxyl end and a second 2′-3′ cyclic phosphate end is performed by introns; and/or
(iv) the RNA ligase ligates an RNA comprising 2′,3′-cyclic phosphate and 5′-OH and/or wherein the RNA ligase is RtcB ligase.

31.-36. (canceled)

37. The method of claim 29, wherein the guide RNA further comprises a linker sequence, and wherein: (i) the linker sequence is between 40 nucleotides and 250 nucleotides; (ii) the linker sequence is placed at the 3′ end and/or the 5′ end of the guide RNA sequence; (iii) the linker sequence is placed only at the 3′ end; (iv) the linker sequence is placed only at the 5′ end; (v) the 3′ end has a longer linker than the linker at the 5′ end, wherein the 3′ end of the guide RNA has a linker that is between 1-20 nucleotides, 5-50 nucleotides, 5-100 nucleotides longer than a linker at the 5′ end of the guide RNA; or (vi) the 5′ end has a longer linker than the linker at the 3′ end, wherein the 5′ end of the guide RNA has a linker that is between 1-20 nucleotides, 5-50 nucleotides, 5-100 nucleotides longer than a linker at the 3′ end of the guide RNA.

38.-45. (canceled)

46. The method of claim 1, wherein: (i) the guide RNA comprises a crRNA, wherein the guide RNA further comprises a tracrRNA, and further wherein the tracrRNA is modified; (ii) the cgRNA is an extended guide RNA, or Cas9 guide RNA, or Cas13 guide RNA, or a Cas12 guide RNA such as Cas12a guide RNA, Cas12b guide RNA, Cas12c guide RNA, Cas12d, guide RNA, Cas12e guide RNA, Cas12f guide RNA, Cas12g guide RNA, Cas12h guide RNA, Cas12i guide RNA, Cas12j guide RNA, Cas12k guide RNA; (iii) the cgRNA comprises one or more of the following: a spacer, a lower stem, a bulge, an upper stem, a nexus and a hairpin; (iv) the cgRNA is produced at a yield of about 50%, 55%, 60%, 65%, 70%, 75%, 80%, 85%, 90%, 95%, 99% or more, wherein the cgRNA is produced at 50%, 55%, 60%, 65%, 70%, 75%, 80%, 85%, 90%, 95%, 99% or more improvement in yield as compared to conventional synthetic methods; (v) the cgRNA has a length of about 40 nucleotides, 100 nucleotides, about 125 nucleotides, about 150 nucleotides, about 175 nucleotides, about 200 nucleotides, or greater than about 200 nucleotides; (vi) the cgRNA comprises one or more backbone modifications, wherein the one or more backbone modifications comprises a 2′ O-methyl or a phosphorothioate modification; or wherein the one or more backbone modifications is selected from 2′-O-methyl 3′-phosphorothioate, 2′O-methyl, 2′-ribo 3′-phosphorothioate, deoxy, 5′ phosphate, 2′fluoro, 2′-O-methoxyethyl (MOE), morpholino (PMO), or locked nucleic acids (LNA) modification; wherein the cgRNA is produced at a quantity of about 100 μg, 200 μg, 300 μg, 400 μg, 500 μg, 600 μg, 700 μg, 800 μg, 900 μg, or 1,000 μg; (vii) the method produces cgRNA at a purity of about 50%, 60%, 70%, 80%, 90%, 95%, 97%, 98%, or more than 99%; (x) the cgRNA is suitable for use with CRISPR/Cas systems, wherein the cgRNA is suitable for use with Cas9, Cpf1, SaCas9, Cas12, Cas13, or modified versions thereof or wherein the cgRNA is in complex with Cas9, Cpf1, SaCas9, Cas12, Cas13, or modified versions thereof; (xi) the cgRNA provides increased stability in comparison to linear guide RNA, wherein the cgRNA provides increased editing events in target cells using a CRISPR/Cas editing system or wherein the linear guide RNA has end modifications; or (xii) the cgRNA has increased resistance to exonuclease in comparison to a linear guide RNA.

47.-70. (canceled)

71. A composition comprising a circularized guide RNA (cgRNA), the cgRNA comprising one or more of a spacer, a lower stem, a bulge, an upper stem, a nexus and a hairpin, and wherein (i) the cgRNA is in a complex with Cas9, Cpf1, SaCas9, Cas12, Cas13, or modified versions thereof; or (ii) the cgRNA is produced by contacting a linear guide RNA with a ligating enzyme, wherein the contacting brings together a first end and a second end of the guide RNA, and wherein the ligating enzyme ligates the first and the second end of the guide RNA thus creating the cgRNA, wherein the cgRNA has increased resistance to exonuclease in comparison to a linear guide RNA, and/or wherein the linear guide RNA has end modifications.

72.-75. (canceled)

76. A Cas protein complex, the complex comprising a Cas nuclease and a circularized gRNA of claim 71, wherein the Cas nuclease is selected from Cas9, Cpf1, SaCas9, Cas12, Cas13, or modified versions thereof.

77. (canceled)

78. A method for targeted transcription activation, targeted transcription repression, targeted epigenome modification, or targeted genome modification, the method comprising introducing into a eukaryotic cell:
(a) a circular guide RNA (cgRNA) as defined in claim 71;
(b) at least one CRISPR/Cas protein or a nucleic acid encoding at least one CRISPR/Cas protein;
wherein interactions between (a) and (b) and a target sequence in chromosomal DNA leads to targeted transcription activation, targeted transcription repression, targeted epigenome modification, or targeted genome modification.

79. A method for targeted RNA modification, the method comprising introducing into a eukaryotic cell:
(a) a circular guide RNA (cgRNA) as defined in claim 71;
(b) at least one CRISPR/Cas protein or a nucleic acid encoding the at least one CRISPR/Cas protein;
wherein interactions between (a) and (b) and an RNA expressed by chromosomal DNA leads to a modification of the RNA expressed by the chromosomal DNA, wherein the RNA expressed by the chromosomal DNA is a messenger RNA (mRNA).

80. (canceled)

81. A kit comprising the composition of claim 71, the kit further comprising one or more of a ligase, a linker sequence, one or more nucleotide splints, wherein the ligase is a T4 RNA Ligase 2, T4 RNA Ligase 1 or RtcB ligase.

82.-83. (canceled)

84. A method of making circular RNA, wherein the circular RNA is made by (A) contacting a linear RNA with a ligating enzyme, wherein the contacting brings together a first end and a second end of the RNA, and wherein the ligating enzyme ligates the first and the second end of the RNA thus creating a circular RNA, or (B) in vitro transcription in the presence of guanosine monophosphate, generating 5′ triphosphate, followed by circularization by self-splicing introns.

85. The method of claim 84, wherein:
(i) the RNA is messenger RNA;
(ii) the linear RNA comprises a terminal 5′ phosphate;
(iii) the ligating enzyme is selected from the group consisting of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, Splint® Ligase E. coli DNA Ligase, 9°N DNA Ligase, CircLigase, CircLigase II, DNA Ligase I, DNA Ligase III, and DNA Ligase IV; (iv) the ligating occurs in the absence of a template between the first end and the second end of the RNA; (v) the first end and the second end of the RNA has partial complementarity, wherein the partial complementarity between the first end and the second end comprises complementarity of at least 5, 10, 15, or 20 nucleotides; (vi) the ligating enzyme is a single-strand ligase, wherein the single-strand ligase is T4 RNA Ligase 1; (vii) the ligating occurs in the presence of a template between the first end and the second end of the RNA; (viii) the ligating enzyme is T4 RNA Ligase 2; (ix) the RNA is between about 100 to 1000 nucleotides long; or (x) the RNA is between about 1000 to 10,000 nucleotides long.

86.-94. (canceled)

95. The method of claim 84, wherein the RNA is synthesized by in vitro transcription, and wherein (i) the in vitro transcription is carried out in the presence of guanosine monophosphate (GMP) to produce RNA; (ii) the in vitro transcribed RNA is treated with pyrophosphohydrolase (RppH) enzyme; (iii) the in vitro transcribed RNA is treated with a ligase, wherein the ligase is T4 RNA ligase 1 or T4 RNA ligase 2.

96.-102. (canceled)

103. The method of claim 84, wherein the RNA is between about 1000 to 10,000 nucleotides long.

104. (canceled)

105. The method of claim 84, wherein (i) the self-splicing intron is Anabaena intron; (ii) the self-splicing intron is T4 intron; (iii) the self-splicing intron is T4 intron and the T4 intron is modified; (iv) the self-splicing intron is Anabaena intron and comprises SEQ ID NOs: 24 and 25; (v) the self-splicing intron is T4 intron and comprises SEQ ID NOs: 26 and 27; and/or (vi) the self-splicing intron is T4 intron and the T4 intron is modified and the self-splicing intron comprises SEQ ID NO: 28 and 29.

106.-110. (canceled)

111. A composition comprising circular RNA generated by the method of claim 84 or a kit comprising the composition comprising circular RNA generated by the method of claim 111.

112. (canceled)
